(19) United States
(12) Patent Application Publication
(10) Pub. No.: US 2023/0417775 A1
(43) Pub. Date: Dec. 28, 2023

(54) NOVEL STEROID HORMONE LIGAND ASSAYS

(71) Applicant: InsituGen Limited, Dunedin (NZ)

(72) Inventor: Alison Kay Heather, Cambridge (NZ)

(21) Appl. No.: 18/036,594

(22) PCT Filed: Nov. 12, 2021
(86) PCT No.: PCT/NZ2021/050201

§ 371 Date: May 11, 2023

Related U.S. Application Data
(60) Provisional application No. 63/112,924, filed on Nov. 12, 2020.

(51) Int. Cl. G01N 33/74; C12Q 1/6897

(52) U.S. Cl. CPC G01N 33/743; C12Q 1/6897; G01N 2333/723

(57) ABSTRACT

The present invention is concerned with the detection of ligands which bind to and activate steroid hormone receptors. Specifically, the present invention provides test kits and assay methods for the selective identification of steroid hormone receptor ligands from a test sample. Importantly, the test kits and assay methods described herein are cell-free, and do not require expensive-to-manufacture nuclear extracts for their performance. Instead, the test kits and assay methods described herein employ single polypeptide polymerases, such as T7 RNA polymerase, linked to a reporter construct. Activity of the enzyme is inhibited, rather than activated, by ligand-bound steroid hormone receptor complexes which only form in the presence of a target ligand. Accordingly, a measured change in a physical property of the reporter construct (e.g. fluorescence output) may be used to determine the presence of a target ligand in a sample under investigation.

### TECHNICAL FIELD

The invention relates generally to the detection of steroid hormone ligand(s) from a sample. In particular, the present invention provides assays, methods and test kits sufficient to screen a test sample for the presence of a ligand as characterized by its ability to form a complex with a steroid hormone receptor and elicit a steroid hormone specific genomic response, for example, by driving transcription of a reporter construct in vitro.

### BACKGROUND OF THE INVENTION

The detection of ligands capable of eliciting a steroid hormone genomic response is important in many areas of biochemistry, molecular biology and medicine. Such ligands include endogenous steroids, exogenous steroids, non-steroidal and synthetic molecules. For example, the determination of total hormone bioactivity in serum or plasma is important for monitoring human and animal health related conditions including aging, perimenopause, menopause, hypoandrogenism, hyperandrogenism, hormone replacement therapy, endocrine cancers including breast and prostate cancers, other hormone related conditions such as osteoporosis and liver toxicity, irregular menstruation, polycystic ovary syndrome, disorders of sexual development and infertility. Conventional detection methods for androgenic/estrogenic and antiandrogenic/antiestrogenic molecules provide no information about hormone biological activity. Hormone biological activity is an important measurement for understanding underlying mechanisms that are driving health conditions so that appropriate treatments/interventions can be implemented.

The detection of hormonal bioactivity in samples is also important for monitoring illicit human and animal performance enhancement, injury cover-up, supplement and food adulteration, growth promoters in dairy industry and environmental pollutants. Measuring hormonal bioactivity provides information about contaminants and/or adulterants that are likely to modulate endocrine pathways in the body, thereby affecting human and animal health.

Ligands that elicit a steroid hormone genomic response first activate steroid hormone receptor proteins by forming a complex with them in the cytoplasm or nucleus of eukaryote cells to form an activated receptor protein. The ligand displaces coregulators that act to stabilise the inactivated receptor protein, which then exposes DNA binding motifs. The activated receptor protein dimerises with a second activated receptor protein and translocates to the nucleus to interact with DNA by binding to a specific nucleotide sequence called a response element. In normal biological function, the assemblage of ligand-activated steroid hormone receptor proteins bound to a response element regulates gene expression by enhancing or repressing the initiation of RNA polymerase II mediated transcription. In its natural state, the activated receptor may recruit other coregulator proteins to either stabilize its DNA binding and/or to help engage RNA polymerase II. RNA polymerase II is a multi-subunit holoenzyme that assembles to catalyse RNA transcription by polymerising nucleotide triphosphates against a DNA template.

The steroid hormone genomic response is induced by ligands that bind to steroid hormone receptor proteins and receptor-specific response elements for example androgen receptor (AR) and the androgen response element (ARE), estrogen receptor-α (ER-α), estrogen receptor-β (ER-β) and the estrogen response element (ERE), glucocorticoid receptor (GR) and the glucocorticoid response element (GRE), mineralocorticoid receptor (MR) and the mineralocorticoid response element (MRE), progesterone receptor-A (PR-A), progesterone receptor-B (PR-B) and the progesterone response element (PRE).

However, not all ligands that bind to steroid hormone receptor proteins elicit a steroid hormone genomic response. Some ligands elicit a non-genomic response that is characterised by second messenger signalling, such as G-protein activation. Such non-genomic responses occur within seconds to minutes of ligand binding, and are not a classical steroid hormone response.

A common way to detect the presence of a ligand in a sample is to measure it directly in that sample. However, samples are often complex mixtures of molecules and typically require a complicated process of preparation for analysis. Detecting the presence of a ligand(s) in a sample typically relies on processes such as liquid or gas chromatography to separate the molecular species from a complex mixture into fractions of relatively pure composition and then analyse each fraction with a structure-sensitive method such as mass spectrometry. More than 100 ligands can be tested in any one sample using this approach. Automated purification systems, gas or liquid chromatograms, and mass spectrometers are costly and technically complicated laboratory instruments that must be continually calibrated and operated by trained technicians in order to produce reliable results. Another disadvantage is that some ligands may be rendered biologically inactive by interaction with proteins such as sex hormone binding globulin or serum albumin and this methodology does not distinguish between biologically active and inactive fractions of ligands. Also, the process of ionization can lead to disintegration of some steroid molecules such that they cannot be measured using such methodologies. Additionally, this methodology does not provide information about the total biological activity of a sample from multiple ligands when all known ligands cannot be identified or where ligands may be identified it is not known if the activity would be additive, synergistic, or even competitive. Furthermore, prior knowledge of the molecular structure of the ligand(s) and its associated metabolite(s) due to the biological metabolism of the ligand(s) is required to achieve reliable identification of the presence of ligand(s) in the sample.

Another common way to detect the presence of a steroid hormone ligand in a sample is to use biological assays based on immunological techniques, such as radioimmunoassay and enzyme-linked immunosorbent assay. A limitation of immunological techniques is the requirement for antibody molecules to detect the ligands directly or the ligands bound to sex hormone binding globulin. Immunological assays lack reproducibility due to the high degree of variability in the antibody molecules produced by different manufacturers of the assays.

To overcome various limitations associated with detection of ligands in a sample (e.g.) requirement for knowledge of the compound structure and/or to provide complex detection reagents such as antibodies, Applicants have developed various generations of in vitro bioactivity assays involving enzyme- or fluorescence-mediated reporter read-outs. These assays mimic biological systems by assembling, in vitro, essential components of in vivo hormone signalling to facilitate detection of a target ligand from a test sample. The essential assay components include, for example, a steroid hormone receptor, steroid hormone receptor coregulator(s) and a reporter construct which includes a specific DNA binding motif which is only bound/activated in the presence of a ligand of interest. For example, the test kits, assays and methods described in PCT/NZ2020/050045 and PCT/NZ2020/050046.

More recently, Applicants have undertaken further optimization work to establish improvements in the configuration and performance of test kits, assays and methods as previously described. For example, improvements in reporter construct architecture and/or the inclusion of additional coregulators which enhance assay specificity and/or sensitivity.

Further, manufacturing improvements in terms of cell lysate production of steroid hormone receptor sufficient to meet scale-up requirements have been developed. Not only does this approach reduce the total cost of goods required for manufacture of test kits, it also creates flexibility with respect to the recombinant production of variant/designer steroid hormone receptors for wider application of the test kits, assays and methods described herein to steroid hormone biology, and commercial applications therein.

Accordingly, the present invention is concerned with these non-obvious improvements.

### SUMMARY OF THE INVENTION

The inventions described and claimed herein have many attributes and examples including, but not limited to, those set forth or described or referenced in this Summary of the Invention. It is not intended to be all-inclusive and the inventions described and claimed herein are not limited to or by the features or examples identified in this Summary of the Invention, which is included for purposes of illustration only and not restriction.

In an aspect of the present invention there is provided a test kit for screening a sample for the presence of a ligand capable of eliciting a steroid hormone genomic response, the test kit comprising:
(i) a cell lysate comprising a steroid hormone receptor that is capable of forming a ligand-receptor complex with a ligand from the sample; and
(ii) a nucleic acid molecule comprising:
   (a) a RNA polymerase promoter sequence;
   (b) a response element that is capable of being bound by the ligand receptor complex; and
   (c) a reporter construct
   wherein the response element (b) is located between the promoter sequence (a) and the reporter construct (c),
   and wherein the nucleic acid molecule optionally comprises a spacer sequence (e) located between the polymerase promoter sequence (a) and the response element (b),
   provided that [(a), (b) and (c)] or [(a), (e), (b) and (c)] are operably linked; and
(iii) optionally, a RNA polymerase, for example a single polypeptide RNA polymerase.

In yet another aspect of the present invention there is provided a test kit for screening a sample for the presence of a ligand capable of eliciting a steroid hormone genomic response, the test kit comprising:
(i) a cell lysate comprising a steroid hormone receptor that is capable of forming a ligand-receptor complex with a ligand from the sample; and
(ii) a nucleic acid molecule comprising:
   (a) a T7 RNA polymerase promoter sequence;
   (b) a response element that is capable of being bound by the ligand receptor complex; and
   (c) a reporter construct
   wherein the response element (b) is located between the promoter sequence (a) and the reporter construct (c),
   and wherein the nucleic acid molecule optionally comprises a spacer sequence (e) located between the polymerase promoter sequence (a) and the response element (b),
   provided that [(a), (b) and (c)] or [(a), (e), (b) and (c)] are operably linked; and
(iii) optionally, a T7 RNA polymerase.

In yet another aspect of the present invention there is provided a test kit for screening a sample for the presence of a ligand capable of eliciting a steroid hormone genomic response, the test kit comprising:
(i) a cell lysate comprising a steroid hormone receptor that is capable of forming a ligand-receptor complex with a ligand from the sample; and
(ii) a nucleic acid molecule comprising:
   (a) a T7 RNA polymerase promoter sequence comprising or consisting in a sequence defined by SEQ ID NO: 85;
   (b) a response element that is capable of being bound by the ligand receptor complex; and
   (c) a reporter construct
   wherein the response element (b) is located between the promoter sequence (a) and the reporter construct (c),
   and wherein the nucleic acid molecule optionally comprises a spacer sequence (e) located between the polymerase promoter sequence (a) and the response element (b),
   provided that [(a), (b) and (c)] or [(a), (e), (b) and (c)] are operably linked; and
(iii) optionally, a T7 RNA polymerase.

In yet another aspect of the present invention there is provided a test kit for screening a sample for the presence of a ligand capable of eliciting a steroid hormone genomic response, the test kit comprising:
(i) a cell lysate comprising a steroid hormone receptor that is capable of forming a ligand-receptor complex with a ligand from the sample; and
(ii) at least one steroid hormone receptor coactivator; and/or
(iii) at least one steroid hormone receptor corepressor; and
(iv) a nucleic acid molecule comprising:
   (a) a RNA polymerase promoter sequence;
   (b) a response element that is capable of being bound by the ligand receptor complex;
   (c) a reporter construct; and
   (d) optionally, at least one binding site that is capable of being bound by at least one coactivator protein, ERG,
   wherein the response element (b) is located between the promoter sequence (a) and the reporter construct (c),
   and wherein the binding site (d) is located immediately upstream (d5′), immediately downstream (d3′), or both immediately upstream (d5′) and immediately downstream (d3′) of the response element (b),
   and wherein the nucleic acid molecule optionally comprises a spacer sequence (e) located between the polymerase promoter sequence (a) and the response element (b)
   provided that [(a), (b) and (c)], [(a), (e), (b) and (c)], [(a), (d5′), (b) and (c)], [(a), (b), (d3′) and (c)], [(a), (e), (d5′), (b) and (c)], [(a), (e), (b), (d3′) and (c)] or [(a), (e), (d5′), (b), (d3′) and (c)] are operably linked; and
(v) optionally, a RNA polymerase, for example a single polypeptide RNA polymerase.

In yet another aspect of the present invention there is provided a test kit for screening a sample for the presence of a ligand capable of eliciting a steroid hormone genomic response, the test kit comprising:
(i) a cell lysate comprising a steroid hormone receptor that is capable of forming a ligand-receptor complex with a ligand from the sample; and
(ii) at least one steroid hormone receptor coactivator; and/or
(iii) at least one steroid hormone receptor corepressor; and
(iv) a nucleic acid molecule comprising:
   (a) a T7 RNA polymerase promoter sequence;
   (b) a response element that is capable of being bound by the ligand receptor complex;
   (c) a reporter construct; and
   (d) optionally, at least one binding site that is capable of being bound by at least one coactivator protein, ERG,
   wherein the response element (b) is located between the promoter sequence (a) and the reporter construct (c),
   and wherein the binding site (d) is located immediately upstream (d5′), immediately downstream (d3′), or both immediately upstream (d5′) and immediately downstream (d3′) of the response element (b),
   and wherein the nucleic acid molecule optionally comprises a spacer sequence (e) located between the polymerase promoter sequence (a) and the response element (b)
   provided that [(a), (b) and (c)], [(a), (e), (b) and (c)], [(a), (d5′), (b) and (c)], [(a), (b), (d3′) and (c)], [(a), (e), (d5′), (b) and (c)], [(a), (e), (b), (d3′) and (c)] or [(a), (e), (d5′), (b), (d3′) and (c)] are operably linked; and
(v) optionally, a T7 RNA polymerase.

In yet another aspect of the present invention there is provided a test kit for screening a sample for the presence of a ligand capable of eliciting a steroid hormone genomic response, the test kit comprising:
(i) a cell lysate comprising a steroid hormone receptor that is capable of forming a ligand-receptor complex with a ligand from the sample; and
(ii) at least one steroid hormone receptor coactivator; and/or
(iii) at least one steroid hormone receptor corepressor; and
(iv) a nucleic acid molecule comprising:
   (a) a T7 RNA polymerase promoter sequence comprising or consisting in the sequence defined by SEQ ID NO: 85;
   (b) a response element that is capable of being bound by the ligand receptor complex;
   (c) a reporter construct; and
   (d) optionally, at least one binding site that is capable of being bound by at least one coactivator protein, ERG
   wherein the response element (b) is located between the promoter sequence (a) and the reporter construct (c),
   and wherein the binding site (d) is located immediately upstream (d5′), immediately downstream (d3′), or both immediately upstream (d5′) and immediately downstream (d3′) of the response element (b),
   and wherein the nucleic acid molecule optionally comprises a spacer sequence (e) located between the polymerase promoter sequence (a) and the response element (b),
   provided that [(a), (b) and (c)], [(a), (e), (b) and (c)], [(a), (d5′), (b) and (c)], [(a), (b), (d3′) and (c)], [(a), (e), (d5′), (b) and (c)], [(a), (e), (b), (d3′) and (c)] or [(a), (e), (d5′), (b), (d3′) and (c)] are operably linked; and
(v) optionally, a T7 RNA polymerase.

In yet another aspect of the present invention there is provided a test kit for screening a sample for the presence of a ligand capable of eliciting a steroid hormone genomic response, the test kit comprising:
(i) a steroid hormone receptor that is capable of forming a ligand-receptor complex with a ligand from the sample; and
(ii) a nucleic acid molecule comprising:
   (a) a T7 RNA polymerase promoter sequence comprising or consisting in SEQ ID NO: 85;
   (b) a response element that is capable of being bound by the ligand receptor complex; and
   (c) a reporter construct
   where the response element (b) is located between the promoter sequence (a) and the reporter construct (c);
and wherein the nucleic acid molecule optionally comprises a spacer sequence (e) located between the polymerase promoter sequence (a) and the response element (b),
   and [(a), (b) and (c)] and [(a), (e), (b) and (c)] are operably linked; and
(iii) optionally, a T7 RNA polymerase.

In yet another aspect of the present invention there is provided a test kit for screening a sample for the presence of a ligand capable of eliciting a steroid hormone genomic response, the test kit comprising:
(i) a steroid hormone receptor that is capable of forming a ligand-receptor complex with a ligand from the sample; and
(ii) at least one steroid hormone receptor coactivator; and/or
(iii) at least one steroid hormone receptor corepressor; and
(iv) a nucleic acid molecule comprising:
   (a) a RNA polymerase promoter sequence, optionally comprising or consisting in SEQ ID NO: 85;
   (b) a response element that is capable of being bound by the ligand receptor complex;
   (c) a reporter construct; and
   (d) optionally, at least one binding site that is capable of being bound by at least one coactivator protein, ERG,
   wherein the response element (b) is located between the promoter sequence (a) and the reporter construct (c),
   and wherein the binding site (d) is located immediately upstream (d5′), immediately downstream (d3′), or both immediately upstream (d5′) and immediately downstream (d3′) of the response element (b),
   and wherein the nucleic acid molecule comprises optionally comprises a spacer sequence (e) located between the polymerase promoter sequence (a) and the response element (b),
   provided that [(a), (b) and (c)], [(a), (e), (b) and (c)], [(a), (d5′), (b) and (c)], [(a), (b), (d3′) and (c)], [(a), (e), (d5′), (b) and (c)], [(a), (e), (b), (d3′) and (c)] or [(a), (e), (d5′), (b), (d3′) and (c)] are operably linked; and
(v) optionally, a T7 RNA polymerase.

In yet another aspect of the present invention there is provided an assay method for detecting a ligand in a sample which ligand is capable of eliciting a steroid hormone genomic response, the assay method comprising the steps of:
(1) contacting a sample with:
   (i) a cell lysate comprising a steroid hormone receptor that forms a ligand-receptor complex with a ligand from the sample; and
   (ii) a nucleic acid molecule comprising:
      (a) a RNA polymerase promoter sequence;
      (b) a response element that is bound by the ligand-receptor complex; and
      (c) a reporter construct
      wherein the response element (b) is located between the promoter sequence (a) and the reporter construct (c),
      and wherein the nucleic acid molecule optionally comprises a spacer sequence (e) located between the polymerase promoter sequence (a) and the response element (b),
      provided that [(a), (b) and (c)] or [(a), (e), (b) and (c)] are operably linked; and
   (iii) a RNA polymerase, for example a single polypeptide RNA polymerase; and
   (iv) ribonucleoside triphosphates; and
(2) measuring a reduction or inhibition in transcription of the reporter construct caused by binding of the ligand-receptor complex to the response element,
wherein, a measured reduction or inhibition in transcription of the reporter construct reflects detection of a ligand in the sample.

In yet another aspect of the present invention there is provided an assay method for detecting a ligand in a sample which ligand is capable of eliciting a steroid hormone genomic response, the assay method comprising the steps of:
(1) contacting a sample with:
   (i) a cell lysate comprising a steroid hormone receptor that forms a ligand-receptor complex with a ligand from the sample; and
   (ii) a nucleic acid molecule comprising:
      (a) a T7 RNA polymerase promoter sequence;
      (b) a response element that is bound by the ligand-receptor complex; and
      (c) a reporter construct
      wherein the response element (b) is located between the promoter sequence (a) and the reporter construct (c),
      and wherein the nucleic acid molecule optionally comprises a spacer sequence (e) located between the polymerase promoter sequence (a) and the response element (b),
      provided that [(a), (b) and (c)] or [(a), (e), (b) and (c)] are operably linked; and
   (iii) a T7 RNA polymerase; and
   (iv) ribonucleoside triphosphates; and
(2) measuring a reduction or inhibition in transcription of the reporter construct caused by binding of the ligand-receptor complex to the response element,
wherein, a measured reduction or inhibition in transcription of the reporter construct reflects detection of a ligand in the sample.

In yet another aspect of the present invention there is provided an assay method for detecting a ligand in a sample which ligand is capable of eliciting a steroid hormone genomic response, the assay method comprising the steps of:
(1) contacting a sample with:
   (i) a cell lysate comprising a steroid hormone receptor that forms a ligand-receptor complex with a ligand from the sample; and
   (ii) a nucleic acid molecule comprising:
      (a) a T7 RNA polymerase promoter sequence comprising or consisting in a sequence defined by SEQ ID NO: 85;
      (b) a response element that is bound by the ligand-receptor complex; and
      (c) a reporter construct
      wherein the response element (b) is located between the promoter sequence (a) and the reporter construct (c),
      and wherein the nucleic acid molecule optionally comprises a spacer sequence (e) located between the polymerase promoter sequence (a) and the response element (b),
      provided that [(a), (b) and (c)] or [(a), (e), (b) and (c)] are operably linked; and
   (iii) a T7 RNA polymerase; and
   (iv) ribonucleoside triphosphates; and
(2) measuring a reduction or inhibition in transcription of the reporter construct caused by binding of the ligand-receptor complex to the response element,
wherein, a measured reduction or inhibition in transcription of the reporter construct reflects detection of a ligand in the sample.

In yet another aspect of the present invention there is provided an assay method for detecting a ligand in a sample which ligand is capable of eliciting a steroid hormone genomic response, the assay method comprising the steps of:
(1) contacting a sample with:
   (i) a cell lysate comprising a steroid hormone receptor that forms a ligand-receptor complex with a ligand from the sample; and
   (ii) at least one steroid hormone receptor coactivator; and/or
   (iii) at least one steroid hormone receptor corepressor; and
   (iv) a nucleic acid molecule comprising:
      (a) a RNA polymerase promoter sequence;
      (b) a response element that is capable of being bound by the ligand receptor complex;
      (c) a reporter construct; and
      (d) optionally, at least one binding site that is capable of being bound by at least one coactivator protein, ERG,
      wherein the response element (b) is located between the promoter sequence (a) and the reporter construct (c),
      and wherein the binding site (d) is located immediately upstream (d5′), immediately downstream (d3′), or both immediately upstream (d5′) and immediately downstream (d3′) of the response element (b),
      and wherein the nucleic acid molecule optionally comprises a spacer sequence (e) located between the polymerase promoter sequence (a) and the response element (b)
      provided that [(a), (b) and (c)], [(a), (e), (b) and (c)], [(a), (d5′), (b) and (c)], [(a), (b), (d3′) and (c)], [(a), (e), (d5′), (b) and (c)], [(a), (e), (b), (d3′) and (c)] or [(a), (e), (d5′), (b), (d3′) and (c)] are operably linked; and
   (v) a RNA polymerase, for example a single polypeptide RNA polymerase; and
   (vi) ribonucleoside triphosphates; and
(2) measuring a reduction or inhibition in transcription of the reporter construct caused by binding of the ligand-receptor complex to the response element,
wherein, a measured reduction or inhibition in transcription of the reporter construct reflects detection of a ligand in the sample.

In yet another aspect of the present invention there is provided an assay method for detecting a ligand in a sample which ligand is capable of eliciting a steroid hormone genomic response, the assay method comprising the steps of:
(1) contacting a sample with:
   (i) a cell lysate comprising a steroid hormone receptor that forms a ligand-receptor complex with a ligand from the sample; and
   (ii) at least one steroid hormone receptor coactivator; and/or
   (iii) at least one steroid hormone receptor corepressor; and
   (iv) a nucleic acid molecule comprising:
      (a) a T7 RNA polymerase promoter sequence;
      (b) a response element that is capable of being bound by the ligand receptor complex;
      (c) a reporter construct; and
      (d) optionally, at least one binding site that is capable of being bound by at least one coactivator protein, ERG
      wherein the response element (b) is located between the promoter sequence (a) and the reporter construct (c),
      and wherein the binding site (d) is located immediately upstream (d5′), immediately downstream (d3′), or both immediately upstream (d5′) and immediately downstream (d3′) of the response element (b),
      and wherein the nucleic acid molecule optionally comprises a spacer sequence (e) located between the polymerase promoter sequence (a) and the response element (b)
      provided that [(a), (b) and (c)], [(a), (e), (b) and (c)], [(a), (d5′), (b) and (c)], [(a), (b), (d3′) and (c)], [(a), (e), (d5′), (b) and (c)], [(a), (e), (b), (d3′) and (c)] or [(a), (e), (d5′), (b), (d3′) and (c)] are operably linked; and
   (v) a T7 RNA polymerase; and
   (vi) ribonucleoside triphosphates; and
(2) measuring a reduction or inhibition in transcription of the reporter construct caused by binding of the ligand-receptor complex to the response element,
wherein, a measured reduction or inhibition in transcription of the reporter construct reflects detection of a ligand in the sample.

In yet another aspect of the present invention there is provided an assay method for detecting a ligand in a sample which ligand is capable of eliciting a steroid hormone genomic response, the assay method comprising the steps of:
(1) contacting a sample with:
   (i) a cell lysate comprising a steroid hormone receptor that forms a ligand-receptor complex with a ligand from the sample; and
   (ii) at least one steroid hormone receptor coactivator; and/or
   (iii) at least one steroid hormone receptor corepressor; and
   (iv) a nucleic acid molecule comprising:
      (a) a T7 RNA polymerase promoter sequence comprising or consisting in a sequence defined by SEQ ID NO: 85;
      (b) a response element that is capable of being bound by the ligand receptor complex;
      (c) a reporter construct; and
      (d) optionally, at least one binding site that is capable of being bound by at least one coactivator protein, ERG
      wherein the response element (b) is located between the promoter sequence (a) and the reporter construct (c),
      and wherein the binding site (d) is located immediately upstream (d5′), immediately downstream (d3′), or both immediately upstream (d5′) and immediately downstream (d3′) of the response element (b),
      and wherein the nucleic acid molecule optionally comprises a spacer sequence (e) located between the polymerase promoter sequence (a) and the response element (b)
      provided that [(a), (b) and (c)], [(a), (e), (b) and (c)], [(a), (d5′), (b) and (c)], [(a), (b), (d3′) and (c)], [(a), (e), (d5′), (b) and (c)], [(a), (e), (b), (d3′) and (c)] or [(a), (e), (d5′), (b), (d3′) and (c)] are operably linked; and
   (v) a T7 RNA polymerase; and
   (vi) ribonucleoside triphosphates; and
(2) measuring a reduction or inhibition in transcription of the reporter construct caused by binding of the ligand-receptor complex to the response element,
wherein, a measured reduction or inhibition in transcription of the reporter construct reflects detection of a ligand in the sample.

In yet another aspect of the present invention there is provided an assay method for detecting a ligand in a sample which ligand is capable of eliciting a steroid hormone genomic response, the assay method comprising the steps of:
(1) contacting a sample with:
   (i) steroid hormone receptor that forms a ligand-receptor complex with a ligand from the sample; and
   (ii) a nucleic acid molecule comprising:
      (a) a T7 RNA polymerase promoter sequence comprising or consisting in the sequence defined by SEQ ID NO: 85;
      (b) a response element that is bound by the ligand-receptor complex; and
      (c) a reporter construct
      wherein the response element (b) is located between the promoter sequence (a) and the reporter construct (c),
      and wherein the nucleic acid molecule optionally comprises a spacer sequence (e) located between the polymerase promoter sequence (a) and the response element (b),
      and [(a), (b) and (c)] or [(a), (e), (b) and (c) are operably linked; and
   (iii) a T7 RNA polymerase; and
   (iv) ribonucleoside triphosphates; and
(2) measuring a reduction or inhibition in transcription of the reporter construct caused by binding of the ligand-receptor complex to the response element,
wherein, a measured reduction or inhibition in transcription of the reporter construct reflects detection of a ligand in the sample.

In yet another aspect of the present invention there is provided an assay method for detecting a ligand in a sample which ligand is capable of eliciting a steroid hormone genomic response, the assay method comprising the steps of:
(1) contacting a sample with:
   (i) steroid hormone receptor that forms a ligand-receptor complex with a ligand from the sample; and
   (ii) at least one steroid hormone receptor coactivator; and/or
   (iii) at least one steroid hormone receptor corepressor; and
   (iv) a nucleic acid molecule comprising:
      (a) a RNA polymerase promoter sequence, optionally comprising or consisting in SEQ ID NO: 85;
      (b) a response element that is bound by the ligand-receptor complex;
      (c) a reporter construct; and
      (d) optionally, at least one binding site that is capable of being bound by at least one coactivator protein, ERG,
      wherein the response element (b) is located between the promoter sequence (a) and the reporter construct (c),
      and wherein the binding site (d) is located immediately upstream (d5′), immediately downstream (d3′), or both immediately upstream (d5′) and immediately downstream (d3′) of the response element (b),
      and wherein the nucleic acid molecule optionally comprises a spacer sequence (e) located between the polymerase promoter sequence (a) and the response element (b),
      provided that [(a), (b) and (c)], [(a), (e), (b) and (c)], [(a), (d5′), (b) and (c)], [(a), (b), (d3′) and (c)], [(a), (e), (d5′), (b) and (c)], [(a), (e), (b), (d3′) and (c)] or [(a), (e), (d5′), (b), (d3′) and (c)] are operably linked; and
   (v) a RNA polymerase; and
   (vi) ribonucleoside triphosphates; and
(2) measuring a reduction or inhibition in transcription of the reporter construct caused by binding of the ligand-receptor complex to the response element,
wherein, a measured reduction or inhibition in transcription of the reporter construct reflects detection of a ligand in the sample.

In another aspect of the present invention there is provided a test kit for screening a sample for the side-by-side detection of an androgenic ligand and/or an estrogenic ligand, the test kit comprising:
(i) a cell lysate comprising an androgen receptor, wherein the androgen receptor is capable of forming an androgen receptor-ligand complex with a complimentary ligand from the sample; and
(ii) a first nucleic acid molecule comprising:
   (a) a polymerase promoter sequence;
   (b) an androgen response element that is capable of being bound by the androgen receptor-ligand complex; and
   (c) a first reporter construct;
   where the response element (b) is located between the promoter sequence (a) and the reporter construct (c), and (a), (b) and (c) are operably linked; and
(iii) a cell lysate comprising an estrogen receptor, wherein the estrogen receptor is capable of forming an estrogen receptor-ligand complex with a complimentary ligand from the sample; and
(iv) a second nucleic acid molecule comprising:
   (d) the polymerase promoter sequence;
   (e) an estrogen response element that is capable of being bound by the estrogen receptor-ligand complex; and
   (f) a second reporter construct; and
(iv) optionally, a single polypeptide polymerase
wherein, the first and second reporter constructs are different.

In other aspects of the present invention there is provided a test kit for screening a sample for the presence of a ligand, which ligand is capable of forming a complex with a steroid hormone receptor and eliciting a genomic response, the test kit comprising:
(i) a cell lysate comprising an androgen receptor that is capable of forming a ligand-receptor complex with a ligand from the test sample; or
(ii) a cell lysate comprising an estrogen receptor that is capable of forming a ligand-receptor complex with a ligand from the test sample, wherein the estrogen receptor is estrogen receptor alpha or estrogen receptor beta; or
(iii) a cell lysate comprising a progesterone receptor that is capable of forming a ligand-receptor complex with a ligand from the test sample, wherein the progesterone receptor is progesterone receptor A or progesterone receptor B; or
(iv) a cell lysate comprising a mineralocorticoid receptor that is capable of forming a ligand-receptor complex with a ligand from the test sample; or
(v) a cell lysate comprising a glucocorticoid receptor that is capable of forming a ligand-receptor complex with a ligand from the test sample; and
(vi) a nucleic acid molecule comprising:
   (a) a T7 RNA polymerase promoter sequence comprising or consisting in SEQ ID NO: 85;
   (b) a response element that is capable of being bound by the receptor-ligand complex; and
   (c) a reporter construct
   where the response element (b) is located between the promoter sequence (a) and the reporter construct (c), and (a), (b) and (c) are operably linked; and
(vii) optionally, a T7 RNA polymerase.

In another aspect of the present invention there is provided a test kit for screening a sample for the presence of a ligand, which ligand is capable of forming a complex with a steroid hormone receptor and eliciting a genomic response, the test kit comprising:
(i) a cell lysate comprising an androgen receptor that is capable of forming a ligand-receptor complex with a ligand from the test sample; or
(ii) a cell lysate comprising a progesterone receptor that is capable of forming a ligand-receptor complex with a ligand from the test sample, wherein the progesterone receptor is progesterone receptor A or progesterone receptor B; or
(iii) a cell lysate comprising a mineralocorticoid receptor that is capable of forming a ligand-receptor complex with a ligand from the test sample; or
(iv) a cell lysate comprising a glucocorticoid receptor that is capable of forming a ligand-receptor complex with a ligand from the test sample; and
(v) optionally, at least one steroid hormone receptor coactivator protein and/or at least one steroid hormone receptor corepressor; and
(vi) a nucleic acid molecule comprising:
   (a) a T7 polymerase promoter sequence comprising or consisting in SEQ ID NO: 85;
   (b) a response element that is capable of being bound by the receptor-ligand complex; and
   (c) a reporter construct
   where the response element (b) is located between the promoter sequence (a) and the reporter construct (c), and (a), (b) and (c) are operably linked; and
(vii) optionally, a T7 RNA polymerase.

In another aspect of the present invention there is provided a test kit for screening a sample for the presence of a ligand, which ligand is capable of forming a complex with an estrogen receptor and eliciting a genomic response, the test kit comprising:
(i) a cell lysate comprising at least one estrogen receptor that is capable of forming a ligand-receptor complex with a ligand from the test sample; and
(ii) optionally at least one steroid hormone receptor coactivator and/or at least one steroid hormone receptor coactivator; and
(iii) a nucleic acid molecule comprising:
   (a) a T7 RNA polymerase promoter sequence comprising or consisting in SEQ ID NO: 85;
   (b) a response element that is capable of being bound by the receptor-ligand complex; and
   (c) a reporter construct
   where the response element (b) is located between the promoter sequence (a) and the reporter construct (c), and (a), (b) and (c) are operably linked; and
(iv) optionally, a T7 RNA polymerase.

In yet another aspect of the present invention there is provided a test kit for the side-by-side detection of an androgenic ligand and/or an estrogenic ligand from a sample, the test kit comprising:
(i) a cell lysate comprising an androgen receptor and an estrogen receptor, wherein the androgen receptor forms a ligand-receptor complex with an androgenic ligand from the sample and/or the estrogen receptor forms a ligand-receptor complex with an estrogenic ligand from the sample; and
(ii) optionally, at least one steroid hormone receptor coactivator; and/or
(iii) optionally, at least one steroid hormone receptor corepressor; and
(iv) a nucleic acid molecule comprising:
   (a) a RNA polymerase promoter sequence;
   (b) a response element that is capable of being bound by the receptor-ligand complex; and
   (c) a reporter construct
   where the response element (b) is located between the promoter sequence (a) and the reporter construct (c), and (a), (b) and (c) are operably linked; and
(v) optionally, a RNA polymerase.

In yet another aspect of the present invention there is provided a test kit to determine the total estrogenic activity of a sample, the test kit comprising:
(i) a cell lysate comprising estrogen receptor alpha and estrogen receptor beta;
(ii) optionally, at least one steroid hormone receptor coactivator; and/or
(iii) optionally, at least one steroid hormone receptor corepressor; and
(iv) a nucleic acid molecule comprising:
   (a) a RNA polymerase promoter sequence;
   (b) a response element that is capable of being bound by the receptor-ligand complex; and
   (c) a reporter construct
   where the response element (b) is located between the promoter sequence (a) and the reporter construct (c), and (a), (b) and (c) are operably linked; and
(v) optionally, a RNA polymerase.

In an example according to this aspect of the present invention, the RNA polymerase is T7 RNA polymerase, and the RNA polymerase promoter sequence is T7 RNA polymerase promoter sequence defined by SEQ ID NO: 85.

In another example according to these and other aspects of the present invention, the nucleic acid sequence optionally comprises a spacer (e) which is located between the promoter sequence (a) and the response element (b). In a related example, the spacer is between about 2 and about 32 nucleotides in length.

In yet another aspect of the present invention there is provided an assay method for detecting a ligand in a sample which ligand is capable of eliciting a steroid hormone genomic response, the method comprising the steps of:
(i) contacting a sample with a test kit comprising a fluorescence-based reporter construct as described herein; and
(ii) measuring a reduction or inhibition in fluorescence of the reporter construct caused by binding of the ligand-receptor complex to the response element, wherein, a measured reduction or inhibition in fluorescence of the reporter construct reflects detection of a ligand in the sample.

In a further aspect of the present invention there is provided a method for determining the steroid hormone bioactivity of a sample, the method comprising combining a sample with a test kit as described herein to ascertain if the sample comprises a ligand sufficient to activate a steroid hormone receptor and cause a change in a physical property of the reporter construct, wherein a change in a physical property of the reporter construct provides information about the steroid hormone bioactivity of the sample.

In a further aspect of the present invention there is provided a method for determining the steroid hormone bioactivity of a biological sample, the method comprising combining a biological sample with a test kit as described herein to ascertain if the sample comprises a ligand sufficient to activate a steroid hormone receptor and cause a change in a physical property of the reporter construct, wherein a change in a physical property of the reporter construct provides information about the steroid hormone bioactivity of the biological sample.

In a further aspect of the present invention there is provided a method for determining the steroid hormone bioactivity of a clinical specimen, the method comprising combining a sample obtained from the clinical specimen with a test kit as described herein to ascertain if the sample comprises a ligand sufficient to activate a steroid hormone receptor and cause a change in a physical property of the reporter construct, wherein a change in a physical property of the reporter construct provides information about the steroid hormone bioactivity of the clinical specimen.

In a further aspect of the present invention there is provided a method for determining the steroid hormone bioactivity of a food or a nutritional supplement, the method comprising combining the food or nutritional supplement, or an extract of the food or nutritional supplement, with a test kit as described herein to ascertain if the sample comprises a ligand sufficient to activate a steroid hormone receptor and cause a change in a physical property of the reporter construct, wherein a change in a physical property of the reporter construct provides information about the steroid hormone bioactivity of the food or nutritional supplement.

In a further aspect of the present invention there is provided a method for determining the steroid hormone bioactivity of a sample derived from an environmental source, the method comprising combining a sample obtained from an environmental source with a test kit as described herein to ascertain if the sample comprises a ligand sufficient to activate a steroid hormone receptor and cause a change in a physical property of the reporter construct, wherein a change in a physical property of the reporter construct provides information about the steroid hormone bioactivity of the environmental sample.

In a further aspect of the present invention there is provided a method for determining the doping status of an athlete, the method comprising combining a sample obtained from an athlete with a test kit as described herein to ascertain if the sample comprises a ligand sufficient to activate a steroid hormone receptor and cause a change in a physical property of the reporter construct, wherein a change in a physical property of the reporter construct provides information about the doping status of the athlete.

In a further aspect of the present invention there is provided a method for determining the steroid hormone bioactivity of a sample, the method comprising performing an assay method as described herein on a sample to ascertain if the sample comprises a ligand sufficient to activate a steroid hormone receptor and cause a change in a physical property of the reporter construct, wherein a change in a physical property of the reporter construct provides information about the steroid hormone bioactivity of the sample.

In a further aspect of the present invention there is provided a method for determining the steroid hormone bioactivity of a biological sample, the method comprising performing an assay method as described herein on a sample to ascertain if the sample comprises a ligand sufficient to activate a steroid hormone receptor and cause a change in a physical property of the reporter construct, wherein a change in a physical property of the reporter construct provides information about the steroid hormone bioactivity of the biological sample.

In a further aspect of the present invention there is provided a method for determining the steroid hormone bioactivity of a food or a nutritional supplement, the method comprising performing an assay method as described herein on the food or nutritional supplement, or an extract from the food or nutritional supplement, to ascertain if the food or nutritional supplement comprises a ligand sufficient to activate a steroid hormone receptor and cause a change in a physical property of the reporter construct, wherein a change in a physical property of the reporter construct provides information about the steroid hormone bioactivity of the food or nutritional supplement.

In a further aspect of the present invention there is provided a method for determining the steroid hormone bioactivity of a clinical specimen, the method comprising performing an assay method as described herein on a clinical specimen to ascertain if the sample comprises a ligand sufficient to activate a steroid hormone receptor and cause a change in a physical property of the reporter construct, wherein a change in a physical property of the reporter construct provides information about the steroid hormone bioactivity of the clinical specimen.

In a further aspect of the present invention there is provided a method for determining the steroid hormone bioactivity of a sample derived from an environmental source, the method comprising performing an assay method as described herein on the environmental sample to ascertain if the sample comprises a ligand sufficient to activate a steroid hormone receptor and cause a change in a physical property of the reporter construct, wherein a change in a physical property of the reporter construct provides information about the steroid hormone bioactivity.

In a further aspect of the present invention there is provided a method for determining the doping status of an athlete, the method comprising performing an assay method as described herein on a sample obtained from the athlete to ascertain if the sample comprises a ligand sufficient to activate a steroid hormone receptor and cause a change in a physical property of the reporter construct, wherein a change in a physical property of the reporter construct provides information about the doping status of the athlete.

In a further aspect of the present invention there is provided an article of manufacture for screening a sample for the presence of a ligand, which ligand is capable of eliciting a steroid hormone genomic response, the article of manufacture comprising a test kit as described herein together with instructions for how to detect the presence of a ligand in the sample.

In a further aspect of the present invention there is provided an article of manufacture for screening a biological sample for the presence of a ligand, which ligand is capable of eliciting a steroid hormone genomic response, the article of manufacture comprising a test kit as described herein together with instructions for how to detect the presence of a ligand in the biological sample.

In a further aspect of the present invention there is provided an article of manufacture for screening a food or a nutritional supplement for the presence of a ligand, which ligand is capable of eliciting a steroid hormone genomic response, the article of manufacture comprising a test kit as described herein together with instructions for how to detect the presence of a ligand in the food or nutritional supplement.

In yet a further aspect of the present invention there is provided an article of manufacture for determining the steroid hormone bioactivity of a sample, the article of manufacture comprising a test kit as described herein together with instructions for detecting the steroid hormone bioactivity in a sample, wherein the presence of bioactive ligands in the sample is indicative of steroid hormone bioactivity of the sample.

In yet a further aspect of the present invention there is provided an article of manufacture for determining the steroid hormone bioactivity of a clinical specimen, the article of manufacture comprising a test kit as described herein together with instructions for detecting the steroid hormone bioactivity in a clinical specimen, wherein the presence of bioactive ligands in the clinical specimen is indicative of steroid hormone bioactivity of the clinical specimen.

In yet a further aspect of the present invention there is provided an article of manufacture for determining the steroid hormone bioactivity of an environmental sample, the article of manufacture comprising a test kit as described herein together with instructions for detecting the steroid hormone bioactivity in an environmental sample, wherein the presence of bioactive ligands in the environmental sample is indicative of steroid hormone bioactivity of the environmental sample.

In yet a further aspect of the present invention there is provided an article of manufacture for determining doping in an athlete, the article of manufacture comprising a test kit as described herein together with instructions for detecting the presence of a ligand in a sample derived from the athlete, wherein the presence of the ligand in the sample is indicative of doping in the athlete.

In yet a further aspect of the present invention there is provided a nucleic acid molecule comprising or consisting in the T7 RNA polymerase promoter sequence defined by SEQ ID NO: 85

In yet a further aspect of the present invention there is provided a nucleic acid molecule comprising:
(a) a T7 RNA polymerase promoter sequence comprising or consisting in SEQ ID NO: 85; and
(b) a hormone response element.

In yet a further aspect of the present invention there is provided a nucleic acid molecule comprising:
(c) a T7 RNA polymerase promoter sequence comprising or consisting in SEQ ID NO: 85;
(d) a hormone response element; and
(e) a reporter construct.

### BRIEF DESCRIPTION OF THE FIGURES

FIG. 1 shows a schematic representation of androgen response element (ARE) mediated inhibition of T7 RNA polymerase mediated transcription of a reporter construct comprising Mango II aptamer sequence under control of the T7 promoter.

FIG. 2 shows AR reduces T7-mediated transcription of RNA Mango II aptamer. In vitro transcription reactions were assembled using a T7 promoter-ARE-RNA Mango II aptamer DNA template. Reactions were assembled in 0.5 mL Eppendorf tubes, and initiated by addition of 11.5 ng/mL testosterone. After 2 hours incubation, RNA Mango II aptamer was measured with the addition of TO1-biotin (TO1-B), and increased fluorescence detected using standard fluorimetry. Black column-control reaction with no AR added, Grey columns-reaction with AR added at 100 ng/reaction or 400 ng/reaction.

FIG. 3 shows AR reduces T7-mediated transcription of RNA Mango II aptamer. In vitro transcription reactions were assembled using a T7 promoter-ARE-RNA Mango II aptamer DNA template. Reactions were assembled in 0.5 mL Eppendorf tubes. After a 2 hour incubation, RNA Mango II aptamer was measured with the addition of TO1-B, and increased fluorescence detected using a standard fluorimeter. Black column-control reaction with no AR added, Grey column-reaction with AR added at 100n g/reaction. Dark grey column—reaction with AR added at 100 ng/reaction and activated with 11.5 ng/mL testosterone.

FIG. 4 shows HSP90 blocks AR inhibition of T7-mediated RNA Mango II aptamer expression. In vitro transcription reactions were assembled using the T7 promoter-ARE-RNA Mango II aptamer DNA template. Reactions were assembled in 0.5 mL Eppendorf tubes. After a two-hour incubation, RNA Mango II aptamer was measured with the addition of TO1-B, and increased fluorescence detected using a standard fluorimeter. Black column-control reaction with no AR added, Grey column-reaction with AR added at 100 ng/reaction. Dark grey column—reaction with AR added at 100 ng/reaction and activated with 11.5 ng/mL testosterone. Light grey column-reaction with AR added at 100 ng/reaction and HSP90 at 200 ng/reaction. Darkest grey column-reaction with AR added at 100 ng/reaction and HSP90 at 200 ng/reaction and testosterone added at 11.5 ng/mL.

FIG. 5 shows testosterone-activated AR dose-dependently reduces T7-mediated expression of RNA aptamer, Mango II. The reactions were assembled as described in the text. For these reactions, T7-generated RNA Mango II was evidenced by the ethanol control. An ethanol control is included as this is the diluent for the steroids, testosterone (T) and dihydrotestosterone (DHT). The fluorescence output generated for the ethanol control was arbitrarily assigned 100%, and used as the reference for the ligand-activated reactions. Testosterone at decreasing concentration (across the range of 2.5 mM to 25 μM) was added to activate AR, or alternatively DHT was added at 250 μM. Both T and DHT are endogenous androgens and potent activators of AR. ****p<0.001 (vs ethanol) one-way ANOVA with Dunnett's multiple comparison test.

FIG. 6 shows titration of AR directly influences ΔMango II. The reactions were assembled as described in the text (e.g. see Table 7). For these reactions, T7-generated RNA Mango II was evidenced by the ethanol control (grey columns). An ethanol control is included as this is the diluent for testosterone (T). The fluorescence output generated for the ethanol control was arbitrarily assigned 100%, and used as the reference for the 250 μM testosterone-activated reactions (black columns). Decreasing the concentration of AR from 50 ng to 25 ng to 14.2 ng maintained a significant level of AR blockade (n=5), however the effect size of T7 inhibition decreased relative to 50 ng. Increasing AR concentration to 100 ng (n=2) had no further effect on T7 inhibition. ****p<0.001, ***p=0.002 (vs ethanol) one-way ANOVA with Dunnett's multiple comparison test.

FIG. 7 shows the HSP90:AR ratio influences ΔMango II. The reactions were assembled as described in the text (see Table 8). For these reactions, T7-generated RNA Mango II was evidenced by the ethanol control (grey columns). The fluorescence output generated for the ethanol control was arbitrarily assigned 100%, and used as the reference for the 250 μM testosterone-activated reactions (black columns). The results show that a 1:1 ratio is effective only as AR concentrations increase, with greatest ΔMango II reported when AR concentration is 100 ng. A 2:1 ratio was effective if AR concentration was 50 ng or 100 ng, while a 4:1 ratio allowed AR to operate even with lower AR concentrations while an 8:1 ratio showed decreased effect size on ΔMango II.

FIG. 8 shows a single ARE site is strong enough to reduce T7 transcription. The reactions were assembled as described in the text, except the DNA templates differed. For one set of reaction, a DNA template was used that encoded just a single ARE, while for the second set of reactions, the original 3×ARE DNA template was used (Sequences shown in Table 2). For these reactions, T7-generated RNA Mango II was evidenced by the ethanol control (grey columns). The fluorescence output generated for the ethanol control was arbitrarily assigned 100% and used as the reference for the 250 μM testosterone-activated reactions (black columns). The results show that a single ARE is as effective as the 3×ARE (****p<0.0001, ***p=0.002).

FIG. 9 shows decreasing DNA template concentration reduces ΔMango II. The reactions were assembled as described in the text. For these reactions, T7-generated RNA Mango II was evidenced by the ethanol control (grey columns). The fluorescence output generated for the ethanol control was arbitrarily assigned 100% and used as the reference for the 250 μM testosterone-activated reactions (black columns). The results show that as DNA template concentration dropped below 25 ng per reaction there was a loss in the ability to detect ΔMango II (****p<0.0001, ***p=0.003).

FIG. 10 shows titrating T7 units from 50U to 10U decreases fluorescence below a detection of change threshold. T7 RNA polymerase was titrated such that 50U to 10U of enzyme was added per reaction. The reaction only consisted of the T7 RNA polymerase, the DNA template and the reaction buffer. Notably, at 50U the Mango II generated from the reaction resulted in a fluorescence readout of ˜340000. This reduced nicely to ˜200000 when 40U of enzyme was added. Any further dilution did not generate a measurable change in fluorescence indicating that ˜200000 fluorescence units is the threshold for this reaction.

FIG. 11 shows the threshold for ΔMango II detection. Reactions were established with 50U or 100U of T7 RNA polymerase, DNA template, reaction buffer, 50 ng AR, 100 ng HSP90, and 250 μM testosterone (or ethanol as control). Data shows that when 50U T7 enzyme was added, there was ability to detect ΔMango II however the effect size is relatively small, albeit significant (n=5, *p=0.0155 one way ANOVA with Sidaks post-hoc test). When 100U of T7 enzyme was added, ΔMango II was easily detected with a greater effect size (n=5, ****p<0.0001). The threshold for ΔMango II detection is ˜200000. If output falls below this, interpretation of test results will be difficult.

FIG. 12 shows A single ERE site is strong enough for estradiol-activated ERα to reduce T7 transcription. The reactions were assembled as described in the text. For these reactions, T7-generated RNA Mango II is evidenced by the ethanol control. An ethanol control is included as this is the diluent for the steroid hormone, estradiol (E2). The fluorescence output generated for the ethanol control was arbitrarily assigned 100%, and used as the reference for the estradiol-activated reactions. Estradiol at 5 uM was added to activate ERα. n=3, ****p<0.001 (vs ethanol) One-way ANOVA with Dunnett's multiple comparison test.

FIG. 13 shows the AR/HSP90-ARE assay is able to detect a range of AAS and SARMs. Testosterone (250 μM) was used as the positive reference while ethanol was used as the negative reference and activity set at 100% (dotted green line). All data is normalized to the ethanol control. The class of androgenic molecules is described in the text (Table 10).

FIG. 14 shows RNA polymerase activity is not affected by serum. The reactions were assembled with DNA template (100 ng), buffer, nuclease-free water and for AR reactions, AR (50 ng), HSP90 (100 ng). Equine serum or FCS (10 μl) were added before the reaction was initiated with the addition 100U (or equivalent) T7 RNA polymerase. The reactions were held at 37° C. for 150 mins before Mango II RNA aptamer was detected with TO1-biotin (100 nM). The data shows that the presence of equine serum or FCS had no effect on T7-generation of Mango II.

FIG. 15 shows detection of testosterone in serum samples. Reactions were assembled as described in text with the exception that 13 μl FCS was added that had been spiked with testosterone. Ethanol-spiked FCS was used as the non-activating AR control and the T7 activity was arbitrarily set at 100%. Testosterone showed a dose-dependent suppression of T7-generated Mango II, with a ΔMango II greater for the higher testosterone concentration. p<0.0001 versus ethanol, one-way ANOVA with Sidak's post-hoc comparison.

FIG. 16 shows detection of androgenic activity in urine samples. Reactions were assembled as described in text. Ethanol was used as the negative (vehicle) control for AR activation, while testosterone (250 μM) was used as the positive control. Colt #1 and Colt #2 represent urine samples obtained from two different young male racehorses. Gelding #1 and gelding #2 represent urine samples obtained from two different male castrated horses. Spiked trenbolone represents a gelding urine sample that had been spiked with trenbolone before the deconjugation and extraction steps. T7 activity measured for the ethanol control was arbitrarily set at 100%. Testosterone showed suppression of T7-generated Mango II that was also seen with the colt urine samples and the spiked trenbolone urine sample, however very little suppression of T7 activity was measured for the gelding samples. Geldings have had their testes removed and as these organs are the major source of testosterone production in males, endogenous androgen levels would be expected to be low.

FIG. 17 shows pro-hormone detection using the SHR/SRE assay. Androstenedione was preincubated with S9 liver fraction before the liver S9 fraction reaction was extracted for steroids. The extracted samples were then tested for androgenic activity. Data shows that methanol control represents full T7 activity, that is not affected when AR/HSP90 is added in the absence of an androgen. When the Androstenedione/S9 extracted sample was tested there was strong reduction in fluorescence readout (n=2 independent steroid metabolism/extraction reactions) compared to Androstenedione (n=2 independent no NAD S9 fraction/extraction reactions).

FIG. 18 shows suppression of T7 activity by testosterone-activated recombinant AR versus testosterone-activated AR lysate. The data shows that reactions (assembled as per Table 11) with testosterone-activated recombinant AR (25 ng/reaction) or testosterone-activated AR lysate (25 ng/reaction) both reduced T7 activity, as indicated by decreased fluorescence output. The decrease in fluorescence output is relative to vehicle control (5% ethanol), the diluent for the steroid testosterone.

FIG. 19 shows concentration-dependent suppression of T7 activity by AR lysate. The data shows that reactions (assembled as per Table 11) with increasing concentrations of AR lysate (6.25-100 ng/reaction) reduced T7 activity, as indicated by decreased fluorescence output. The decrease in fluorescence output is relative to vehicle control (5% ethanol), the diluent for the steroid testosterone. The ratio 6.25/25 etc represents AR:HSP90 concentrations used in each reaction.

FIG. 20 shows a comparison of AR lysate batches for their effect on suppressing T7 lysate activity. The data shows that reactions (assembled as per Table 11) using two different batches (#OA741, #011311) of commercially available AR lysate (ORIGENE) reduced T7 activity, as indicated by decreased fluorescence output. The decrease in fluorescence output is relative to vehicle control (5% ethanol), the diluent for the steroid testosterone. AR lysate was used at a concentration of 40 ng/μL. Slope −198656 vs −139234. Inter-variability of n=2, ˜ 30%.

FIG. 21 shows in-house AR lysate preparation suppression of T7 activity. The data shows that reactions (assembled as per Table 11) using an in-house preparation of AR lysate from cultured HEk293 cells reduced T7 activity, as indicated by decreased fluorescence output. The decrease in fluorescence output is relative to vehicle control (5% ethanol), the diluent for the steroid testosterone. The cell lysate was compared to the cytoplasm and nuclear extracts from the same cell preparation. AR lysate and extracts were used at a concentration of 25 ng/μL. Slope of linear regression: whole cell −181093, cytoplasm −137097, and nuclear −104102.

FIG. 22 shows a comparison of six batches of in-house preparations of HEK293 AR cell lysate on suppression of T7 activity. The data shows that reactions (assembled as per Table 11) using in-house preparations of AR lysate from cultured HEk293 cells reduced T7 activity, as indicated by decreased fluorescence output. The in-house AR lysates were compared to recombinant AR (25 ng/μL). The decrease in fluorescence output is relative to vehicle control (5% ethanol), the diluent for the steroid testosterone. AR lysate was used at a concentration of 40 ng/μL. Slope of linear regression: #1 −201331, #2 −159597, #3 −222680, #4 −196456, #5 −148207, #6 −150676, recombinant AR −248851 FIG. 23 shows that AR lysate is not activated by other steroid hormones, estradiol and dexamethasone. The data shows that reactions (assembled as per Table 11) using testosterone-activated AR lysate reduced T7 activity, as indicated by decreased fluorescence output. The AR lysate was not activated by estradiol (1 nM) or dexamethasone (1 μM, 100 nM). The decrease in fluorescence output is relative to vehicle control (5% ethanol for estradiol or 5% acetonitrile). AR lysate was used at a concentration of 40 ng/μL. Slope of linear regression: T −93453, E2+22616, +Dexa 1 μM+22617, +Dexa 100 nM+4309 FIG. 24 shows that AR lysate responds to anabolic androgen steroids (AAS) and selective androgen receptor modulators (SARMs). The data shows that reactions (assembled as per Table 11) using testosterone-, andarine or 11keto-testosterone activated AR lysate reduced T7 activity, as indicated by decreased fluorescence output. The decrease in fluorescence output is relative to vehicle control (5% ethanol). AR lysate was used at a concentration of 40 ng/μL. There was a dose-dependent increase in the slope of linear regression T250 −569498, T125 −426615, and 2.5 mM −873008, 1 mM −538241, 11ketoT250 −430247.

FIG. 25 shows that AR lysate was able to detect androgens in equine plasma. The data shows that reactions (assembled as per Table 11) using plasma androgen-activated AR lysate (ORIGENE OA741) or plasma androgen-activated recombinant AR reduced T7 activity, as indicated by decreased fluorescence output. The decrease in fluorescence output is relative to a commercially available equine serum. AR lysate was used at a concentration of 40 ng/μL and recombinant AR was used at concentration of 25 ng/μL. G44 has higher androgen bioactivity than G33 as determined by a HEK293 cell-based androgen bioassay. G44 shows stronger suppression of T7 activity indicative of higher androgen bioactivity in androgen screening assay, as determined by AR lysate or recombinant AR.

FIG. 26 shows that AR lysate was able to detect androgens in human male serum. The data shows that reactions (assembled as per Table 11) using human male serum androgen-activated AR lysate (ORIGENE OA741) reduced T7 activity, as indicated by decreased fluorescence output. The decrease in fluorescence output is relative to a commercially available human female serum. AR lysate was used at a concentration of 40 ng/μL. Male human serum has higher androgen bioactivity than female human serum, as determined by AR lysate or recombinant AR.

FIG. 27 shows relative T7 activity for constructs comprising T7 promoter sequences defined by SEQ ID Nos: 1, 82 and 83. The data shows that reactions (assembled as per Table 11) using DNA templates that encode different T7 promoter sequences. T7 activity is reported as increased fluorescence. SEQ ID NO: 1 is a 18 bp sequence, SEQ ID NO: 82 represents the wildtype sequence and SEQ ID NO: 83 represents the 3′modification to the wildtype sequence. SEQ ID NO: 1 shows the highest T7 activity, with SEQ ID NO: 82 inferior to SEQ ID NO: 83 by 55%. SEQ ID NO: 83 supports increased T7 activity relative to SEQ ID NO: 82 as reported in the literature.

FIG. 28 shows an evaluation of the different T7 promoter sequences defined by SEQ ID Nos: 1, 82 and 83 for androgen responsiveness. The data shows that reactions (assembled as per Table 11) using DNA templates that encode different T7 promoter sequences. Recombinant AR was activated with testosterone, and the difference from control (5% ethanol) was compared. The decrease in fluorescence output is relative to the control.

FIG. 29 shows the relative T7 activity for the T7 promoter sequences defined by SEQ ID NO: 1 and SEQ ID NO: 84. The data shows that reactions (assembled as per Table 11) using DNA templates that encode different T7 promoter sequences. T7 activity is reported as increased fluorescence.

FIG. 30 shows an evaluation of the different T7 promoter sequences defined by SEQ ID Nos: 1 and 84 for androgen responsiveness. The data shows that reactions (assembled as per Table 11) using DNA templates that encode different T7 promoter sequences. Recombinant AR was activated with testosterone, and the difference from control (5% ethanol) was compared. The decrease in fluorescence output is relative to the control.

FIG. 31 shows the relative T7 activity for the T7 promoter sequences defined by SEQ ID Nos: 1 and 84. The data shows that reactions (assembled as per Table 11) using DNA templates that encode the T7 promoter defined by SEQ ID NO: 1 and SEQ ID NO: 84. Recombinant AR was activated with testosterone, and the difference from control (5% ethanol) was compared. The decrease in fluorescence output is relative to the paired control reactions.

FIG. 32 shows an androgen screening assay, inclusive of a DNA reporter construct comprising the T7 promoter sequence defined by SEQ ID NO: 84, is not activated by other steroid hormones, estradiol and dexamethasone, when recombinant AR is included. The data shows that reactions (assembled as per Table 11) using testosterone-activated recombinant AR reduced T7 activity, as indicated by decreased fluorescence output. Recombinant AR was not activated by estradiol (1 nM) or dexamethasone (1 μM, 100 nM). The decrease in fluorescence output is relative to vehicle control (5% ethanol for estradiol or 5% acetonitrile). Recombinant AR was used at a concentration of 25 ng/μL. Slope of linear regression: T −358043, E2 −150583, +Dexa 1 μM 221551, +Dexa 100 nM −169732.

FIG. 33 shows an androgen screening assay, inclusive of a DNA reporter construct comprising the T7 promoter sequence defined by SEQ ID NO: 84 is not activated by other steroid hormones, estradiol and dexamethasone, when AR lysate is included. The data shows that reactions (assembled as per Table 11) using testosterone-activated AR lysate reduced T7 activity, as indicated by decreased fluorescence output. The AR lysate was not activated by estradiol (1 nM) or dexamethasone (1 μM, 100 nM). The decrease in fluorescence output is relative to vehicle control (5% ethanol for estradiol or 5% acetonitrile). AR lysate was used at a concentration of 40 ng/μL. Slope of linear regression: T −570551, E2 1 nM −252629, E2 0.1 nM 276445+Dexa 1 uM −91645, +Dexa 100 nM 58589.

FIG. 34 shows a time course assay for T7 activity with DNA reporter constructs comprising a T7 promoter defined by SEQ ID NOs: 1 and 84. The data shows that reactions (assembled as per Table 11) with SEQ ID NOs: 1 and 84 and testosterone-activated recombinant AR decreased T7 activity relative to controls, as indicated by decreased fluorescence output over time. The decrease in fluorescence output is relative to vehicle control (5% ethanol). Recombinant AR was used at a concentration of 25 ng/μL. SEQ ID NO: 84 shows higher T7 activity as indicated by the initial hill slope of the logarithmic curve.

FIG. 35 shows a time course assay for T7 activity with a DNA reporter construct comprising a T7 promoter defined by SEQ ID NO: 84 in the presence of 50 ng and 100 ng of this DNA construct. The data shows that reactions (assembled as per Table 11) with SEQ ID NO: 84 at 50 ng and 100 ng and testosterone-activated recombinant AR decreased T7 activity relative to controls, as indicated by decreased fluorescence output over time. Recombinant AR was used at a concentration of 25 ng/μL. 50 ng of T7 promoter defined by SEQ ID NO: 84 shows less T7 activity indicating that there is not excess DNA for T7, and that T7 RNA polymerase enzyme is in excess of DNA template.

FIG. 36 shows a time course assay for T7 activity with DNA reporter constructs comprising a T7 promoter defined by SEQ ID NOs: 1 and 84 in the presence of 25 ng, 50 ng or 100 ng of recombinant AR. The data shows that reactions (assembled as per Table 11) with SEQ ID NO: 84 and recombinant AR at 25 ng, 50 ng and 100 ng. Testosterone-activated AR decreased T7 activity relative to controls, as indicated by decreased fluorescence output over time. AR at 25 ng or 50 ng per reaction did not interfere with T7 activity, with both reactions showing time-dependent increase in fluorescence. AR at 50 ng/reaction showed better separation between testosterone and the control reaction suggesting that the AR:T7/DNA ratio is optimal. If 100 ng AR/reaction is used, there is too much blockade of T7 activity and there is very little T7 activity reported, and the difference between testosterone and control becomes difficult to measure. Notably, the difference between testosterone and the control is evident by 30-40 mins.

FIG. 37 shows ETS increases the dynamic range of the androgen screening assay. Reactions were assembled as per Table 1, with the addition of 12.5 ng ERG protein. Recombinant AR was activated with 250 μM testosterone, relative to the vehicle-control (5% ethanol). In the first reaction, the AR/HSP90 was activated with no ERG protein present. In the second reaction, the AR/HSP90 was activated with ERG protein present. The ERG protein increased the suppression of T7 activity suggesting the stronger interaction of AR with the DNA to inhibit progress of T7 RNA polymerase. (n=1).

FIG. 38 shows a minimum of 3:1 ERG:AR ratio is more effective at blocking T7 activity than AR alone. Reactions were established with DNA sequence 88 (100 ng), T7 RNA polymerase (8 U/μl), T7 reaction buffer, AR (50 ng) and HSP90 (100 ng). AR was activated with testosterone (250 μM) or as vehicle control (5% ethanol). The T7 reactions were incubated at 37° C. for 1 hour after which RNA Mango II was detected by binding to fluorophore thiazole orange-1 (TO-1).

FIG. 39 shows that the length of the spacer between the T7 binding site and the ARE affects androgen detection. This is likely because the T7 RNA polymerase binds to the promoter and then proceeds ˜7 bp. At this point, T7 RNA polymerase can disengage from the DNA template or continue through to the elongation steps. The spacer defined by SEQ ID NO: 86 holds the ARE sequence 15 bp from the T7 promoter sequence, which would allow T7 RNA polymerase to continue through the 7 bp initiation steps and have proceeded into the 15 bp elongation steps. The spacer defined by SEQ ID NO: 74 holds the ARE sequence 12 bp from the T7 promoter which also allows good transcription rates, however, the range for androgen detection is less sensitive compared to a spacer length of 15 bp. SEQ ID NO: 87 encodes a 9 bp spacer that does not support good T7 mediated transcription or androgen detection.

FIG. 40 shows Pc-3 androgen receptor cell lysate can substitute for recombinant AR in the cell-free assay. Reactions were prepared in accordance with Example 10, with AR activated by testosterone (250 μM, black bars), or andarine (1 μM, white bars) with ethanol as vehicle control (set as 100% T7 RNA polymerase activity). Pc-3 lysate was compared to AR-expressing HEK293 cell lysate (40 ng, prepared in-house), commercially sourced HEK293 cell lysate (40 ng, Origene) or recombinant AR protein (40 ng, rAR Creative Biomart). No significant differences were measured (one-way ANOVA with Dunnett's multiple comparison test) between any AR lysate preparation and recombinant AR.

FIG. 41 shows Pc-3 AR lysate can detect testosterone in a dose-dependent manner. Reactions were prepared in accordance with Example 10, with 25 ng Pc-3 AR lysate activated by testosterone at concentrations between 500-31.25 μM with ethanol as vehicle control (set as 100% T7 RNA polymerase activity).

FIG. 42 shows Pc-3 AR lysate can detect a range of androgenic molecules. Reactions were prepared in accordance with Example 10, with 40 ng Pc-3 AR lysate activated testosterone at 250, 125 and 62.5 μM (dose T), or andarine 2.5 and 1 μM (dose A) with ethanol as vehicle control (set as 100% T7 RNA polymerase activity).

FIG. 43 shows Pc-3 AR lysate can detect a range of testosterone preparations. Reactions were prepared in accordance with Example 10, with 40 ng Pc-3 AR lysate activated by testosterone at 250, 125 and 62.5 μM (dose T), or andarine 2.5 and 1 μM (dose A) with ethanol as vehicle control (set as 100% T7 RNA polymerase activity). T=testosterone FIG. 44 shows the effect of spacer length between T7 promoter and AR binding site on the ability to detect testosterone. Reactions were prepared in accordance with Example 11, with recombinant AR (rAR, Creative Biomart) activated by testosterone (250 μM, black bars) and ethanol as vehicle control (set as 100% T7 RNA polymerase activity). The ethanol:testosterone (E:T) T7 RNA polymerase activity as determined by raw fluorescence output from Mango II generation was calculated. A higher E:T ratio shows better efficacy of AR blockade of T7 RNA polymerase. DNA sequences tested were 12 bp, 15 bp, 18 bp, 21 bp, 24 bp, 27 bp and 2 bp spacers (refer to Table 14). One-way ANOVA with Dunnett's multiple comparison test was used for statistical comparisons.

FIG. 45 shows the effect of the ARE flanking or spacer sequence on the androgen screening assay. Reactions were prepared in accordance with Example 12, with recombinant AR (rAR, Creative Biomart) activated by testosterone (250 μM, black bars) and ethanol as vehicle control (set as 100% T7 RNA polymerase activity). The ethanol:testosterone (E:T) T7 RNA polymerase activity as determined by raw fluorescence output from Mango II generation was calculated. A higher E:T ratio shows better efficacy of AR blockade of T7 RNA polymerase. DNA sequences tested were SEQ ID NO: 94 (flanking bp changed to A), SEQ ID NO: 95 (flanking bp changed to T), SEQ ID NO: 96 (5′ ARE spacer set to T), SEQ ID NO: 97 (5′ and 3′ ARE spacer set to T and A, respectively) (refer to Table 15). There was a significant decrease in the T:E ratio for all changes made to the standard ARE sequence and its flanking bps. One-way ANOVA with Dunnett's multiple comparison test was used for the statistical comparisons.

### GENERAL DEFINITIONS

Unless specifically defined otherwise, all technical and scientific terms used herein shall be taken to have the same meaning as commonly understood by one of ordinary skill in the art (for example, in immunology, immunohistochemistry, protein chemistry, molecular genetics, synthetic biology and biochemistry).

It is intended that reference to a range of numbers disclosed herein (e.g. 1 to 10) also incorporates reference to all related numbers within that range (e.g. 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

The term “and/or”, e.g., “X and/or Y” shall be understood to mean either “X and Y” or “X or Y” and shall be taken to provide explicit support for both meanings or for either meaning.

The term “a” or “an” refers to one or more than one of the entity specified; for example, “a receptor” or “a nucleic acid molecule” may refer to one or more receptor or nucleic acid molecule, or at least one receptor or nucleic acid molecule. As such, the terms “a” or “an”, “one or more” and “at least one” can be used interchangeably herein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### Selected Definitions

For the purposes of the present invention, the following terms shall have the following meanings.

The term “test kit” as used herein refers to an article of manufacture comprising various components to perform the assays and methods according to the inventions described herein.

The term “steroid hormone receptor” or “SHR” as used herein refers to a protein or polypeptide, including recombinant polypeptides that selectively binds to a ligand, which ligand is capable of activating the steroid hormone receptor, and includes, without limitation, an androgen receptor, an estrogen receptor, a progesterone receptor, a mineralocorticoid receptor and a glucocorticoid receptor. Typically, a steroid hormone receptor comprises a ligand binding domain, an activation domain and a deoxyribonucleic acid binding domain. According to this definition, “steroid hormone receptor” may optionally include other corepressors, including (e.g.) heat shock proteins and the like, which help to hold the steroid hormone receptor in a folded and hormone responsive state for activation by a ligand.

The term “steroid hormone receptor coregulator” as used herein includes a “steroid hormone receptor activator” and/or a “steroid hormone receptor repressor”.

A “steroid hormone receptor repressor” according to the present invention includes proteins or molecules that hold the steroid hormone receptor in an inactive state, until displaced by a ligand, at which point the steroid hormone receptor becomes activated. Examples of steroid hormone receptor repressors according to the present invention include, without limitation, heat shock protein 90 (HSP90); a complex of HSP90 and heat shock protein 70 (HSP70); a complex of HSP90, HSP70 and heat shock protein 40 (HSP40); a complex of HSP90, HSP70, HSP40 and p23; a complex of HSP90, HSP70, HSP40, p23 and heat shock protein organizing protein (Hop); a complex of HSP90, HSP70, HSP40, p23, Hop and 48 kD Hip protein (Hip); a complex of HSP90, HSP70, HSP40, p23, Hop, Hip and p60; and a complex of HSP90, HSP70, HSP40, p23, Hop, Hip, p60 and FKBP52.

A “steroid hormone receptor activator” according to the present invention includes a protein or molecule that holds the steroid hormone receptor in an active state and/or enhances the binding interaction between an activated steroid hormone receptor and its complimentary response element. Examples of steroid hormone receptor activators according to the present invention include, without limitation, the erythroblast transformation-specific transcription factor ERG; p160 coactivators inclusive of steroid receptor coactivators, SRC-1, SRC-2, SRC-3; Vav3 a Rho GTPase guanine nucleotide exchange factor; E2F1; ATAD2; CBP/p300; Leupaxin; FHL2; the ARA family of proteins; GRIP1; BRAC1; and Zac1. The skilled person will recognise that certain cell types will express certain subtypes of steroid hormone receptor activators, and that these cells may be genetically altered to increase endogenous expression of coactivators or altered to express different types of coactivators depending on the intended utility. The same is also true for receptor type(s).

The term “ligand” refers generally to any molecule that binds to a receptor, and includes without limitation, a steroid, a polypeptide, a protein, a vitamin, a carbohydrate, a glycoprotein, a therapeutic agent, a drug, a glycosaminoglycan, or any combination thereof. As used herein, “ligand” includes, without limitation, steroid hormones, such as sex hormones including but not limited to estrogens, progestagens, androgens etc, as well as natural and synthetic derivatives and analogs and metabolites thereof, designer steroid hormones, anabolic androgenic steroids, and selective androgen-, progestagen- and estrogen receptor modulators, those that are currently known and those anticipated to be developed or naturally found in biological samples. The term “receptor-ligand complex” and “activated steroid hormone receptor” as used interchangeably herein to refer to a ligand bound steroid hormone receptor, where the steroid hormone receptor undergoes a structural transformation upon binding the ligand and is then said to be in an activated form. A receptor-ligand complex as described herein includes, without limitation, a dimer of a ligand bound hormone receptor (i.e. (HR-L)₂.

The term “AAS” as used herein refers to an anabolic androgenic steroid. These compounds are capable of binding to an androgen receptor to form an activated androgen receptor-ligand complex.

The term “SARM” as used herein refers to selective androgen receptor modulator. These compounds are also capable of binding to an androgen receptor to form an activated androgen receptor-ligand complex.

The term “EtOH” as used herein refers to ethanol, and is widely used as a vehicle control in various experiments described herein.

The term “genomic response” as used herein refers to the ability of an activated steroid hormone receptor (or receptor-ligand complex) to selectively bind to its corresponding nucleic acid response element and activate or repress transcription of downstream genes. In the cellular environment, the ligand-bound receptor binds to the nucleic acid response element and switches genes on or off in response to the external stimuli (i.e. presence of a ligand).

The test kits, assays and methods according to the present invention have been developed to identify ligands which, in a cellular environment, would elicit a steroid hormone genomic response by providing reporter based frameworks which mimic aspects of cell based systems.

The term “steroid metabolism machinery” as used herein refers to any enzyme, and includes combinations of enzyme, sufficient to convert a ligand from a physiologically inactive form to a physiologically active form or from a physiologically active form to a more physiologically active form, or from a physiologically active form to a less physiologically active form, or from a physiologically active form to a physiologically inactive form.

The term “detection means” as used herein refers to any apparatus, equipment or configuration adapted to detect the binding interaction between an activated steroid hormone receptor and nucleic acid response element. Examples of detection means include, but are not limited to, optical methods, spectroscopy, visible spectroscopy, Raman spectroscopy, UV spectroscopy, surface plasmon resonance, electrochemical methods, impedance, resistance, capacitance, mechanical sensing by changes in mass, changes in mechanical resonance, electrophoresis, gel electrophoresis, gel retardation, imaging, fluorescence and fluorescence resonance energy transfer, polymerase chain reaction etc.

The term “nucleic acid sequence” as used herein refers to a deoxyribonucleic acid (DNA) sequence, a ribonucleic acid sequence (RNA), messenger ribonucleic acid (mRNA) and complementary DNA (cDNA), and is comprised of a continuous sequence of two or more nucleotides, also referred to as a polynucleotide or oligonucleotide. The nucleic acid sequence may be single-stranded or double-stranded.

The term “reporter construct” as used herein refers to a nucleic acid sequence encoding a reporter molecule that encodes an RNA whose expression may be assayed; such RNA includes, but are not limited to, fluorophore binding aptamers, or synthetic RNA or mRNA, Additionally, reporter genes may encompass any gene of interest whose expression product may be detected by RNA analysis.

The term “promoter” as used herein is a nucleic acid sequence located proximal to the start of transcription at the 5′ end of an operably linked transcribed sequence. The promoter may contain one or more regulatory elements which interact in modulating transcription of an operably linked gene. Examples of promoters according to the present invention include, but are not limited to, T7, T3 and SP6 bacteriophage promoters or initiation sequences. The terms “promoter”, “promoter sequence”, “initiator sequence” or “initiation sequence” may be used interchangeably throughout this specification to mean the same thing.

The term “T7” or “T7 polymerase” as used herein refers to the T7 RNA polymerase enzyme.

The term “operably linked” as used herein describes two macromolecular elements arranged such that modulating the activity of the first element induces an effect on the second element. In this manner, modulation of the activity of a promoter element may be used to alter and/or regulate the expression of an operably-linked reporter construct. For example, the transcription of a reporter construct that is operably-linked to a promoter element is induced by factors that “activate” the promoter's activity; transcription of a reporter construct that is operably-linked to a promoter element is inhibited by factors that “block” the promoter's activity. Thus, a promoter region is operably-linked to the reporter construct if transcription of such a reporter construct is influenced by the activity of the promoter.

The term “expression” as used herein refers to the process by which the information encoded within a gene is expressed. If the gene encodes a protein, expression involves both transcription of the DNA into mRNA, the processing of the mRNA (if necessary) into a mature mRNA product, and translation of the mature mRNA into protein. A nucleic acid molecule, such as a deoxyribonucleic acid (DNA) or gene is said to be “capable of expressing” a polypeptide (or protein) if the molecule contains the coding sequences for the polypeptide and the expression control sequences which, in the appropriate host environment, provide the ability to transcribe, process and translate the genetic information contained in the DNA into a protein product, and if such expression control sequences are operably-linked to the nucleotide sequence that encodes the polypeptide.

The term “sample” as used herein refers to any sample for which it is desired to test for the presence of a ligand. The terms “sample” and “test sample” are used interchangeably in this specification.

The term “relative potency” or “RP” as used herein refers to the multiplier of biological activity exhibited by a test compound relative to a reference compound, where the biological activity is defined by the ability of the compound to bind to and activate a steroid hormone receptor (e.g.) as measured using the assays and test kits described herein. Where Relative Potency is >1, the test compound is more potent in terms of its biological activity as compared to the reference compound; where Relative Potency is <1, the test compound is less potent in terms of its biological activity as compared to the reference compound; and where Relative Potency=1, the test and reference compounds are equally potent in terms of their biological activities.

The term “activation factor” or “AF” as used herein relates to the measure of metabolic conversion of a test compound (e.g.) from a physiologically inactive state to a physiologically active state or from a less physiologically active state to a more physiologically active state. An activation factor >1 means that the test compound has undergone metabolic conversion to a more physiologically active state in the presence of metabolic machinery in the assay.

The term “reference threshold” or “reference standard” may be used interchangeably to means the level of assay activity measured (e.g.) in the absence of a test sample, or in the absence of test sample and steroid hormone receptor. In certain examples according to the inventions described herein, the reference threshold or reference standard is determined using ethanol in place of test sample. The reference threshold is intended to establish any baseline activity or signal of the assay in the absence of target ligand.

### DETAILED DESCRIPTION

The present invention provides test kits, assays and methods useful for screening a sample for the presence of a ligand capable of activating a steroid hormone receptor and eliciting a steroid hormone genomic response.

In certain examples according to the present invention, the test kits, assays and methods described herein are useful for determining the hormone status of a human or animal subject, for example, by measuring the androgenic and/or estrogenic activity of a sample obtained from the subject. This information may then be used to determine, for example, whether the subject has, or is at risk for developing, cancer, or for investigating endocrine issues or for monitoring a changing steroid hormone profile associated with ageing such as, for example, menopause, or for evaluating the efficacy of hormone replacement therapy or hormone inhibitory therapy.

In other examples according to the present invention, the test kits, assays and methods described herein are useful for screening foods and health food supplements for banned additives or for natural activators that could be harmful to health including, but not limited to, phytoestrogens or xenoandrogens or xenoestrogens that could promote hormone sensitive cancers.

### Enzyme Mediated Activity Assays & Test Kits

The assays according to the present invention, on which the test kits and methods described herein are based, are fundamentally activity based assays which work on the principle of steroid hormone receptor activation through binding of a ligand derived from a sample under interrogation. Activation of a steroid hormone receptor occurs when a ligand binds to the receptor and induces a conformational change in the tertiary structure of the receptor protein, meaning that the receptor-ligand complex (also referred to herein as an ‘activated steroid hormone receptor’) is then able to bind to a nucleic acid response element and influence RNA polymerases and elicit a so-called ‘genomic response’. In other words the ability to up- or down-regulate expression of genes from the genome of the cell which may then lead to a physiological effect. It is this binding interaction between the activated steroid hormone receptor and nucleic acid response element that is measured in a reconstituted in vitro system by the test kits, assays and methods described herein, as a proxy to detect the presence of a ligand having steroid-, or steroid-like activity in a sample under investigation.

Importantly, this means the test kits and assays according to the present invention have the ability to detect steroid hormone bio/activity elicited by ligands of unknown structure such as (e.g.) ‘designer drugs’. Historically, this has not been possible since conventional laboratory testing equipment, such as gas/liquid chromatography and mass spectrometry, requires prior knowledge of the structure of the molecule being investigated.

To address limitations associated with prior art assays as previously described, Applicants have developed various generations of in vitro cellular-free bioactivity assays involving enzyme- or fluorescence-mediated reporter read-outs. These assays mimic biological systems by assembling, in vitro, essential components of in vivo hormone signalling to facilitate detection of a target ligand. The essential assay components include, for example, a steroid hormone receptor, steroid hormone receptor coregulator(s) and a reporter construct which includes a specific DNA binding motif which is only bound/activated in the presence of a target ligand.

For example, PCT/NZ2018/050158 describes enzyme-based transcription and/or translation assays involving RNA polymerase II. However, an inherent limitation associated with these assays is the requirement to provide cell extracts derived from (e.g.) mammalian or yeast origin as a source of both enzyme and coregulator(s) necessary to drive transcription or translation of a reporter gene.

The Applicant's next generation assays described in PCT/NZ2020/050046 removed the requirement for cell-derived extracts by employing single subunit polymerases in the reaction mix. For example, T7 RNA polymerase, which unlike RNA polymerase II could be produced recombinantly. As a consequence, the reduced level of molecular complexity meant assay specificity/sensitivity was significantly enhanced because molecular stoichiometries between essential assay components could be precisely controlled.

Notwithstanding the advantages associated with reduced molecular complexity, a limitation of the assays described in PCT/NZ2020/050046 is the requirement to provide purified steroid hormone receptor.

Recombinant steroid hormone receptor protein is expensive to produce in quantities required for scale-up production. It is also difficult to handle due to cold temperature storage requirements, and its large size renders it susceptible to degradation. By way of illustration only, androgen receptor (AR) protein is a large protein of ˜110 kDa. Expression by genetically modified yeast, insect, bacteria or mammalian host cells requires complex purification techniques to purify the steroid hormone receptor from other cellular proteins, which is made more difficult so as to ensure full-length and active protein is recovered. More often than not, expression and purification of steroid hormone receptors is performed in the presence of ligand to enhance protein stability during the laborious manipulations. Accordingly, there is substantial cost to the purchase, or production, of recombinant steroid hormone receptor protein and it may be that ligand is present in the steroid hormone receptor added to the reaction which would lead to inflated hormone levels reported, or indeed a false positive (if testing of an athlete sample).

The present invention, in part, seeks to address these limitations by providing test kits, assays and methods involving a cell lysate derived from naturally occurring cell lines which express certain steroid hormone receptor(s) and/or cell-specific coregulator(s) (e.g. LnCaP cells which express androgen receptors; HeLa cells which express estrogen receptor alpha and estrogen receptor beta) or modified cell lines which have been genetically altered to express steroid hormone receptor protein(s) and/or coregulator(s) of interest, depending on the intended utility. The use of steroid hormone receptor lysates markedly decreases the potential cost of the screening assays because there is no need for extensive and expensive purification steps.

By way of illustration only, the prototype assays used in the experiments referred to immediately below involve T7 polymerase, the activity of which is reduced or inhibited, rather than activated, by ligands that bind to an androgen receptor.

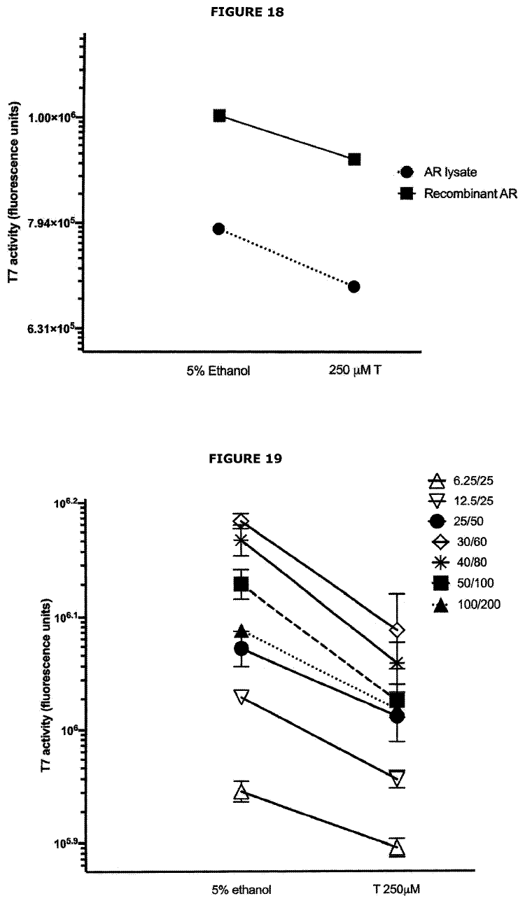

In reference to Example 7, read in conjunction with FIGS. 18-20, Applicants tested two different commercial cell lysates containing androgen receptor in a prototype androgen assay involving T7 polymerase mediated fluorescence read-out of Mango II RNA aptamer (refer to Table 11 for assay components).

These data show that each of the AR cell lysates tested adequately suppressed T7 RNA polymerase activity in the presence of testosterone, a ligand widely known to bind to and activate androgen receptor.

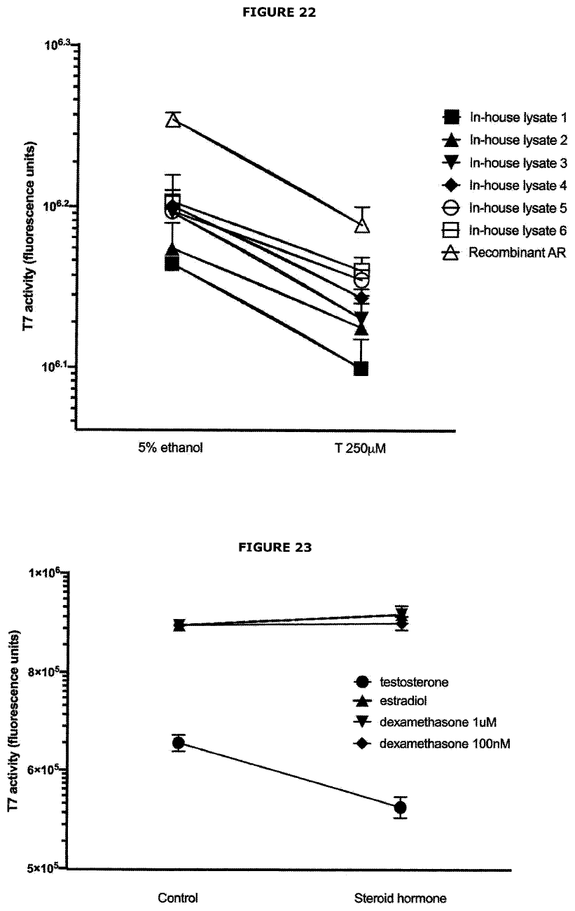

To explore inter-variability in batch production, various in-house AR lysates were then produced from human embryonic kidney cells (e.g. HEK293; refer to Example 8) which had been stably transformed with a human AR expression plasmid. These data are also presented in Example 7, read in conjunction with FIGS. 21-22, and demonstrate that the inter-variability in assay performance was minimal (i.e. 16%). In terms of relative performance, the various AR cell lysate screening assays, performed similarly to an assay which used recombinant androgen receptor (FIG. 22).

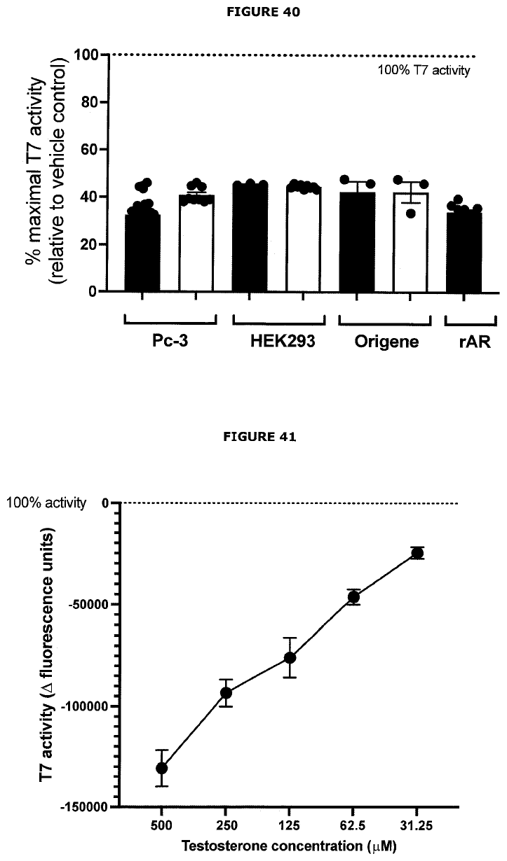

With reference to Example 10 and FIG. 40, Applicants further demonstrate the suitability of cell lysates sourced from alternative cell sources. These data indicate that a cell lysate derived from androgen receptor-expressing Pc-3 cells function efficiently in the in vitro assays described herein and can be used as a substitute for recombinant androgen receptor. By way of illustration only, FIG. 40 shows that androgen receptor-expressing Pc-3 cells were able to invoke a similar level of T7 activity to recombinant AR and cell lysates derived from in-house (“HEK293”) or commercially sourced (“Origene”) HEK293 cells which are known to express androgen receptor.

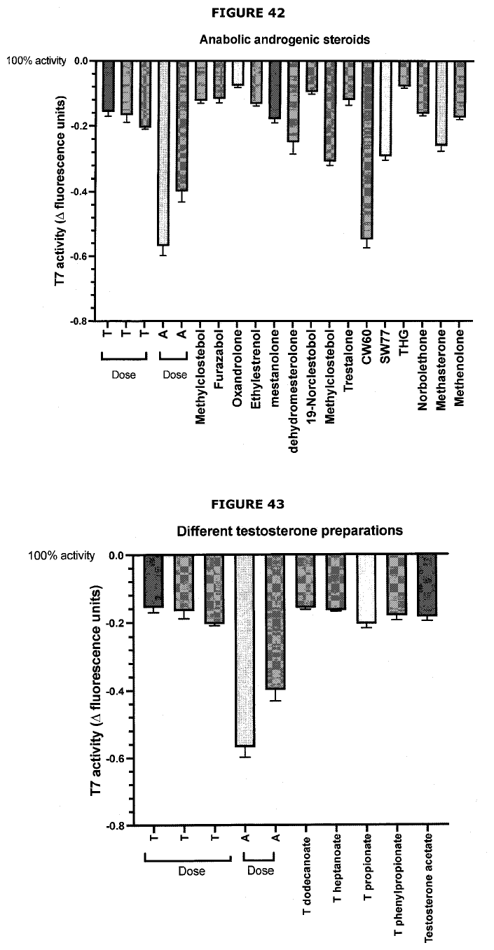

The data presented in FIG. 41 further demonstrate the suitability of the Pc-3 cell lysate as a source of androgen receptor where increased concentrations of testosterone result in lower T7 polymerase activity as measured by reduced fluorescence of the reporter construct used in these experiments. Again, refer to Example 10. Finally, the data presented in FIGS. 42-43 demonstrate the utility of the Pc-3 cell lysate as a source of androgen receptor for detection of a range of anabolic androgen steroids (FIG. 42) and testosterone esters (FIG. 43).

An inherent risk in using a cell lysate as a source of steroid hormone receptor versus a recombinant source of receptor protein is the potential for cross-receptor activation of the hormone response element which is located within the reporter construct. For example, activated glucocorticoid receptor (GR) found within a cell lysate has the potential to bind to and activate an androgen response element within a reporter construct leading to a false positive result for detection of an androgenic ligand. However, the data presented in Example 7, read in conjunction with FIG. 23, demonstrates there was no glucocorticoid receptor activation of the androgen response element in the presence of dexamethasone (i.e. GR ligand) using the HEK293 cell lysates tested.

Another potential confounder in the androgen screening assay is estradiol (E2). At non-physiological concentrations, E2 can activate androgen receptor, although standard cell culture conditions should not produce levels of E2 or any other estrogen sufficient for cross-activation. Indeed, the data presented in Example 7, also read in conjunction with FIG. 23, demonstrate that E2 (1 nM) failed to suppress T7 activity. Accordingly, any reduction in T7 activity observed in the androgen receptor cell lysate assays is due to the presence of androgenic ligands, as confirmed by the comparative testosterone data point in FIG. 23.

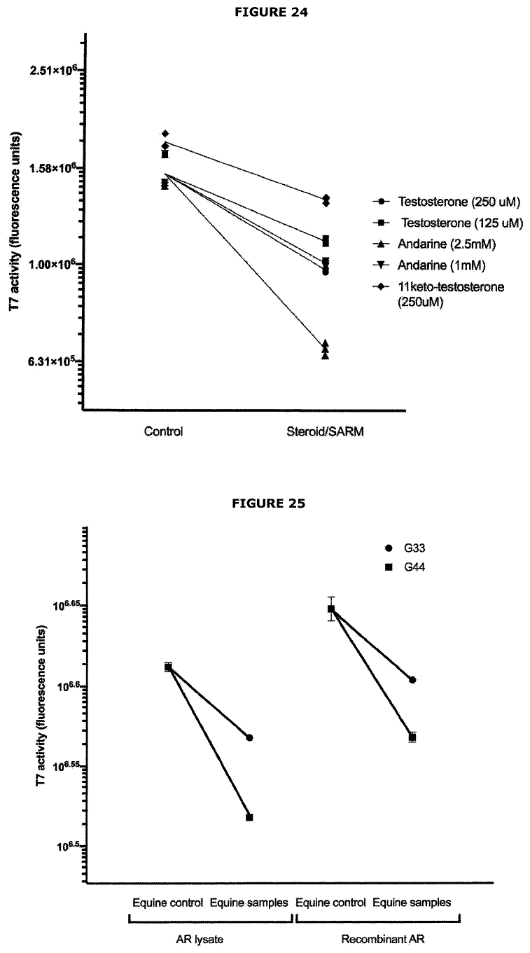

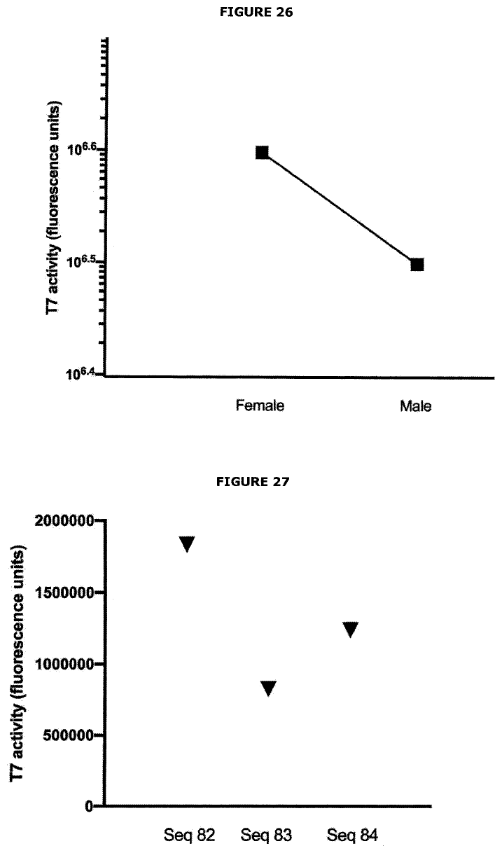

Finally, the remaining data presented in Example 7, read in conjunction with FIGS. 24-26, confirms that the androgen cell lysate screening assays were able to accurately detect the presence of anabolic androgenic steroids (AAS) as well as selective androgen receptor modulators (SARMS) (FIG. 24), and determine androgen bioactivity in equine plasma samples (FIG. 25) and human plasma samples (FIG. 26).

There are numerous advantages to using a cell lysate as a source of steroid hormone receptor protein. In addition to reduced manufacturing costs, the use of cell lysates as a source of steroid hormone receptor protein means a wide variety of host cells may be genetically modified to express different steroid hormone receptor isotypes or variants, depending on the steroid hormone biology under interrogation. For example, by producing cell lysates inclusive of steroid hormone receptor protein which has a superior affinity for its complimentary response element or steroid hormone receptor protein which has superior affinity for its ligand. Accordingly, use of the cell lysates in the test kits and assay methods described could have far reaching clinical application(s) in addition to (e.g.) sports doping utility.

Host cells may also be genetically modified to co-express multiple steroid hormone receptors (e.g. androgen receptor and estrogen receptor) or to co-express desired coregulator proteins which, when present in the cell lysate, advantageously help optimise performance of the test kits and assay methods described herein.

Further, all cell lysates will contain steroid hormone coregulator proteins. In the case of (e.g.) androgen receptor, there are over 30 different coregulator proteins. It has been established in the literature that a subset of these coregulator proteins are ‘core’ and expressed in all cell types. However, another subset of coregulator proteins are specific to cell type. Accordingly, the present invention further contemplates the genetic manipulation of certain cell types to over-express both receptor and coregulator proteins.

While the various experiments referred to above involve validation of a cell lysate approach using an androgen screening assay, the skilled person would appreciate that these principles would apply equally to detection of other steroid hormone ligands, provided a complementary assay framework was established. For example, for the detection of estrogenic ligands, a similar screening assay involving an ER cell lysate (e.g. obtained from a cell transformed with an ER expression plasmid), in combination with a reporter construct comprising an estrogen response element that would only be bound/activated by (e.g.) an E2-estrogen receptor complex.

As evidenced by the data presented in FIGS. 18, 22 and 25, detection of androgenic ligands in a cell lysate screening assay relative to an assay involving recombinant androgen receptor is less sensitive overall. However, and importantly, the cell lysate screening assays still achieve a sufficient level of sensitivity to warrant use, for example, in field applications such as in “dilute and shoot” assays.

Another advantage conferred by the cell lysate screening assays is the ability to select variant/designer steroid hormone receptors for recombinant expression by host cells. In turn this allows wider application of the test kits, assays and methods described herein to steroid hormone biology, and commercial applications therein.

The cell lysate can be prepared from transient or stable expression of a steroid hormone receptor expression plasmid, which will allow the use of a particular receptor in the screening assay that has, for example, as stronger binding affinity for a target ligand. Alternatively, (e.g) in the case of androgen receptor, the receptor could be modified such that it represents androgen insensitivity syndrome, or partial androgen insensitivity syndrome, allowing for a genetic screen to be established.

The cell lysate can be prepared from transient or stable expression of a species-specific steroid hormone receptor expression plasmid, which will allow the expression of for e.g. equine androgen receptor expressed in equine cells. Alternatively, canine androgen receptor expressed in canine cells. This could be of particular use for animal diagnostic applications and/or the detection of designer steroids in animal athlete biological samples.

Another advantage conferred by the cell lysate approach is the ability to prepare co-transformed cells that, for e.g., express steroid hormone receptor and one or more steroid hormone receptor coregulators, such as (e.g.) HSP90 and/or ERG. As established herein, these cofactor proteins help modulate steroid hormone receptor behavior in the various screening assays described herein.

Accordingly, in an aspect of the present invention there is provided a test kit for screening a sample for the presence of a ligand capable of eliciting a steroid hormone genomic response, the test kit comprising:
(i) a cell lysate comprising a steroid hormone receptor that is capable of forming a ligand-receptor complex with a ligand from the sample; and
(ii) a nucleic acid molecule comprising:
   (a) a RNA polymerase promoter sequence;
   (b) a response element that is capable of being bound by the ligand receptor complex; and
   (c) a reporter construct
   where the response element (b) is located between the promoter sequence (a) and the reporter construct (c), and (a), (b) and (c) are operably linked; and
(iii) optionally, a RNA polymerase.

In an example according to this aspect of the present invention, the cell lysate further comprises at least one coregulator protein which includes, by definition, at least one coactivator protein and/or at least one corepressor protein as defined herein.

Applicants also interrogated to what extent the distance between the T7 promoter recognition sequence and the androgen response element influenced assay performance. FIG. 39 demonstrates that placing the androgen response element closer to the promoter sequence may inhibit T7 more substantially by blocking the initiation step. A spacer length of 15 bp (SEQ ID NO: 86) was compared to a spacer length of 9 bp (SEQ ID NO: 87) and spacer length of 12 bp (SEQ ID NO: 88). The 12 bp spacer (SEQ ID NO: 88) was shown to support T7 activity as well as 15 bp spacer (SEQ ID NO: 86), however it was not as efficient in suppressing testosterone-induced blockade of T7 activity (i.e. compare slopes of SEQ ID NO: 88 to SEQ ID NO: 86). The 9 bp spacer (SEQ ID NO: 87) was shown to be inefficient in supporting T7 activity (lower fluorescence output) and testosterone-induced blockade of T7 activity was lost when there was just 9 bp between the 3′ end of the T7 promoter and the ARE. The 15 bp spacer (as well as a 27 bp spacer; refer below) provides adequate space between T7 enzyme binding and AR binding to permit physical blockade.

Further interrogation of optimal spacer length is presented in Example 11 read in conjunction with FIG. 44. Specifically, these data show that spacer lengths of 2 bp, 15 bp and 27 bp yielded higher E:T ratios showing increased efficacy of the AR blockade of T7 RNA polymerase compared with (e.g.) spacer lengths of 12 bp and, to a lesser extent, 18 bp, 21 bp and 24 bp. However, it would be appreciated by a person skilled in the art that spacer lengths of (e.g.) 12 bp, 18 bp, 21 bp and 24 bp still facilitate a sufficient receptor-mediated blockade of T7 RNA polymerase activity, and as such support the definition of a spacer length of between about 2 bp and about 32 bp.

The data presented in FIG. 44 surprisingly demonstrates that a spacer length of 2 bp yielded a significant increase in receptor-mediated blockade of T7 RNA activity relative to the longer spacers. The close proximity of the 110 kDa AR protein is likely physically blocking T7 RNA polymerase from binding to its promoter sequence. When the distance however is 9 bp we see that there is both T7 RNA polymerase transcription and AR blockade issues suggesting that the close proximity allows for some binding of both proteins, however they are likely unstable in their DNA binding due to steric hindrance. A length of 12-15 and greater bp allows a more stable binding of both proteins whereby AR can block T7 RNA polymerase transcription rather than DNA binding.

Accordingly, in an example according to these and other aspects of the present invention, the nucleic acid molecule further comprises a spacer (e) located between the polymerase promoter sequence (a) and the response element (b).

While the data referred to above is true in context of the androgen screening assay described herein, it is possible that screening assays involving a binding interaction between an activated steroid hormone receptor-ligand complex and its response element may differ.

Accordingly, Applicants demonstrate that the spacer may comprise a nucleic acid (i.e. DNA) sequence that is between about 2 nucleotides and about 32 nucleotides in length. For any avoidance of doubt the term “between about 2 nucleotides and about 32 nucleotides in length” is intended to mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 and 32 nucleotides in length. A person skilled in the art will appreciate the term “about” in the context of “between about 2 nucleotides and about 32 nucleotides in length” does not exclude a spacer length that is (e.g.) 33, 34 or 35 or more nucleotides in length. The data presented in Example 11 teaches the skilled person how to determine the optimal spacer length for any given reporter construct defined herein, without the requirement for undue experimentation or further inventiveness.

In a related example, the spacer is 2 nucleotides in length.

In a further related example, the spacer is 15 nucleotides in length.

In another related example, the spacer is 27 nucleotides in length.

It is important to note that the role of the spacer is a physical barrier between the RNA polymerase promoter sequence and the steroid hormone response element, such that T7 RNA polymerase is impeded at the crucial structural reorganizational step whereby RNA polymerase transitions from the initiation (binding) step to the elongation step. T7 RNA polymerase enters the highly processive elongation step after the nascent RNA is between 8-12 nucleotides in length. It is optimal to have the spacer length such that it is short enough to not allow T7 RNA polymerase to achieve full transitioning to elongation but large enough to allow two large proteins (T7 RNAP and the SHR) to bind to the template DNA and allow the bound SHR to sterically hinder active T7 RNAP or to have the spacer length such that is short enough to not allow T7 RNA polymerase to bind to its promoter sequence. This is achieved by ensuring the reaction mix is made in an ordered way such that the AR/HSP90 and DNA template and sample are added prior to T7 RNA polymerase. This temporal separation of AR to T7 RNA polymerase allows AR to first bind the DNA template and thereby block T7 RNA polymerase access to its binding site.

Further, the sequence composition of the spacer is inconsequential to its function and may contain any natural or non-naturally occurring nucleotides known in the art.

It would be considered routine optimisation, based on the teaching provided herein, for the skilled person to determine the optimal spacer length based on a particular construct/assay architecture (e.g. if the assay was configured to employ a T3 polymerase which binds to a T3 promoter sequence).

In another example according to these and other aspects of the present invention, the test kit further comprises ribonucleotide triphosphates (NTPs).

In yet another example according to these and other aspects of the present invention, the test kit further comprises instructions for how to determine the presence of ligand in the sample which is capable of eliciting a steroid hormone genomic response.

Also contemplated by the present invention is methods for the detection of steroid hormone ligands by measuring a reduction or inhibition in transcription using the test kit framework/s described above.

Accordingly, in yet another aspect of the present invention there is provided an assay method for detecting a ligand in a sample which ligand is capable of eliciting a steroid hormone genomic response, the assay method comprising the steps of:
(1) contacting a sample with:
   (i) a cell lysate comprising a steroid hormone receptor that forms a ligand-receptor complex with a ligand from the sample; and
   (ii) a nucleic acid molecule comprising:
      (a) a RNA polymerase promoter sequence;
      (b) a response element that is bound by the ligand-receptor complex; and
      (c) a reporter construct
         where the response element (b) is located between the promoter sequence (a) and the reporter construct (c), and (a), (b) and (c) are operably linked;
   (iii) a RNA polymerase; and
   (iv) ribonucleoside triphosphates; and
(2) measuring a reduction or inhibition in transcription of the reporter construct caused by binding of the ligand-receptor complex to the response element,
wherein, a measured reduction or inhibition in transcription of the reporter construct reflects detection of a ligand in the sample.

Again, in an example according to this aspect of the present invention, the cell lysate further comprises at least one coregulator protein which includes, by definition, at least one coactivator protein and/or at least one corepressor protein as defined herein.

The use of a cell lysate as a source of steroid hormone receptor, as opposed to (e.g.) purified/recombinant steroid hormone receptor means that the absolute concentration of receptor in the reaction mix is not known. However, an optimal cell lysate concentration may be empirically derived. For example, with reference to Example 7 read in conjunction with FIG. 19, the androgen receptor cell lysate (i.e. AR cell lysate) was titrated from 150 ng/μL to 6.25 ng/μL. The data presented in FIG. 19 shows the optimal concentration of AR cell lysate to adequately suppress T7 RNA polymerase activity in the presence of an androgenic ligand was 40 ng/μL, with an optimum concentration range of 30 to 50 ng/μL. However, even across the titration range, a measurable suppression is T7 activity was observed in the presence of testosterone.

Accordingly, in a further example according to the test kits, assays and methods described herein, the concentration of steroid hormone receptor is between about 1.0 ng/μL and about 300 ng/μL, in particular between about 5 ng/μL and about 200 ng/μL, in particular between about 6.25 ng/μL and about 150 ng/μL, in particular between about 10 ng/μL and about 100 ng/μL, in particular between about 20 ng/μL and about 80 ng/μL, in particular between about 30 ng/μL and about 70 ng/μL, in particular between about 30 ng/μL and about 60 ng/μL, in particular between about 30 ng/μL and about 50 ng/μL, in particular about 40 ng/μL.

The skilled person would appreciate that the term “between about 10 ng/μL and about 100 ng/μL” includes, without limitation, about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and about 100 ng/μL, and includes by definition the terms “between about 20 ng/μL and about 80 ng/μL”, “between about 30 ng/μL and about 70 ng/μL”, “between about 30 ng/μL and about 60 ng/μL”, and “between about 30 ng/μL and about 50 ng/μL”.

Accordingly, in yet another example according to the test kits and assay methods described herein:
(i) the test kit comprises an androgen receptor, and the concentration of androgen receptor is between about 10 ng/μL and about 100 ng/μL, including without limitation about 10, 15, 20, 25, 30, 35, 40, 45, 50, 55, 60, 65, 70, 75, 80, 85, 90, 95 and about 100 ng/μL;
(ii) the test kit comprises an estrogen receptor, and the concentration of estrogen receptor is between about 10 ng/μL and about 100 ng/μL, including without limitation about 10, 15, 20, 25, 30, 35, 40, 45, 50, 55, 60, 65, 70, 75, 80, 85, 90, 95 and about 100 ng/μL;
(iii) the test kit comprises a progesterone receptor, and the concentration of progesterone receptor is between about 10 ng/μL and about 100 ng/μL, including without limitation about 10, 15, 20, 25, 30, 35, 40, 45, 50, 55, 60, 65, 70, 75, 80, 85, 90, 95 and about 100 ng/μL;
(iv) the test kit comprises a mineralocorticoid receptor, and the concentration of mineralocorticoid receptor is between about 10 ng/μL and about 100 ng/μL, including without limitation about 10, 15, 20, 25, 30, 35, 40, 45, 50, 55, 60, 65, 70, 75, 80, 85, 90, 95 and about 100 ng/μL; and
(v) the test kit comprises a glucocorticoid receptor, and the concentration of glucocorticoid receptor is between about 10 ng/μL and about 100 ng/μL, including without limitation about 10, 15, 20, 25, 30, 35, 40, 45, 50, 55, 60, 65, 70, 75, 80, 85, 90, 95 and about 100 ng/μL.

Notwithstanding the above considerations, care must be taken not to saturate the assay with too much receptor, since this in itself may create thermodynamic or kinetic barriers that prevent optimal binding of a ligand-receptor complex to its complimentary response element. According to the disclosure provided herein, the skilled person could undertake routine experimentation to titrate an optimized concentration/range of cell lysate comprising steroid hormone receptor for performance of the test kits and assay methods described herein (e.g. refer to Example 7 which follows).

As previously mentioned, Applicants have further determined that the steroid hormone receptor retains some capacity to bind to and activate its corresponding nucleic acid response element in the absence of a ligand specific for its steroid hormone receptor. This phenomena is sometimes referred to as auto-activation of the nucleic acid response element. Accordingly, it may be desirable to determine a reference threshold (i.e. baseline signal as a result of auto-activation of the response element) in the absence of ligand to assist with a determination of absolute assay signal/readout in the presence of a sample containing a target ligand.

Accordingly, in another example according to the test kits and assay methods described herein, the reduction or inhibition in transcription of the reporter construct is measured relative to a reference threshold.

In yet another example according to the test kits and assay methods described herein, reduction or inhibition in transcription of the reporter construct is measured relative to a reference threshold as determined by measuring transcription of the reporter construct in the absence of a test sample.

In yet a further example according to the test kits and assay methods described herein, reduction or inhibition in transcription of the reporter construct is measured relative to a reference threshold as determined by measuring transcription of the reporter construct in the absence of a test sample and in the absence of receptor.

In a parallel approach to enhance assay specificity and performance, the test kit and assay methods described herein may be modified to include at least one steroid hormone receptor coregulator protein or molecule. The coregulator protein or molecule may be provided by the cell lysate per se, or it may be included as a separate integer of the kit (e.g.) a protein or molecule that has been produced using recombinant expression and/or purified, and packaged separately within the kit.

The primary purpose of the coregulator protein or molecule is to either (i) hold the steroid hormone receptor in an inactive conformation, thereby preventing it from binding to and activating the hormone response element in the absence of a target ligand (i.e. through activity of at least one steroid hormone receptor corepressor) or (ii) hold the steroid hormone receptor in an active state and/or enhance the binding interaction between an activated steroid hormone receptor and its complimentary response element (i.e. through activity of at least one steroid hormone receptor coactivator).

Accordingly, in an example according to these and other aspects of the present invention, the steroid hormone receptor coregulator comprises a steroid hormone receptor corepressor. In a related example, the steroid hormone receptor corepressor comprises at least one of heat shock protein 90 (HSP90); a complex of HSP90 and heat shock protein 70 (HSP70); a complex of HSP90, HSP70 and heat shock protein 40 (HSP40); a complex of HSP90, HSP70, HSP40 and p23; a complex of HSP90, HSP70, HSP40, p23 and heat shock protein organizing protein (Hop); a complex of HSP90, HSP70, HSP40, p23, Hop and 48 kD Hip protein (Hip); a complex of HSP90, HSP70, HSP40, p23, Hop, Hip and p60; a complex of HSP90, HSP70, HSP40, p23, Hop, Hip, p60 and FKBP52; and any combination thereof.

When the test kit is contacted with a test sample, the presence of a ligand causes displacement of the corepressor and the ligand-bound receptor is then free to form a complex with a second ligand-bound receptor and bind its complementary hormone response element. The latter may be stabilised by activity of the coactivator protein or molecule.

Accordingly, in another example according to these and other aspects of the present invention, the steroid hormone receptor coregulator comprises a steroid hormone receptor activator. In a related example, the steroid hormone receptor activator comprises at least one of erythroblast transformation-specific transcription factor ERG; p160 coactivators inclusive of steroid receptor coactivators, SRC-1, SRC-2, SRC-3; Vav3 a Rho GTPase guanine nucleotide exchange factor; E2F1; ATAD2; CBP/p300; Leupaxin; FHL2; the ARA family of proteins; the prostate-cell specific coactivators including GRIP1, BRAC1 and Zac1.

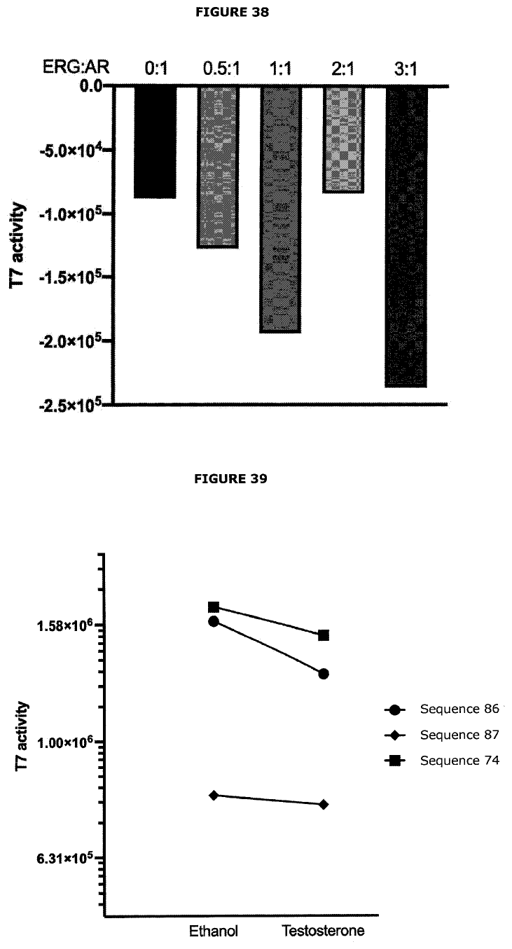

Indeed, the effect of the erythroblast transformation-specific transcription factor ERG is well documented by the data presented in FIGS. 37 and 38. Specifically, the data presented in FIG. 37 demonstrates a significantly enhanced reduction in fluorescence relative to an experiment with no ERG present in the reaction mix.

In this experiment, a molar ratio of 1:1 ERG:AR was used, or expressed as a ratio of protein concentration: 25 ng AR: 12.5 ng ETS, with 50 ng HSP90.

Applicants then titrated the amount of ERG in the reaction mix, and these data are presented in FIG. 38. Specifically, a ratio of 0:5:1 ERG:AR, 1:1 ERG:AR, 2:1 ERG:AR and 3:1 ERG:AR was used in the T7 reaction mixture. The concentrations of AR and HSP90 were held constant at 50 ng and 100 ng respectively. The results demonstrate that the addition of ERG protein improved the reaction, with a data anomaly observed for the 2:1 ratio. However, overall the data shows that a 3:1 ratio of ERG:AR led to greater suppression of T7 activity than the other ratios tested. Applicants are not able to discount at this time if ratios >3:1 may lead to superior suppression in fluorescence output.

In respect of the class II steroid hormone receptors (i.e. including androgen receptor, progesterone A receptor, progesterone B receptor, mineralocorticoid receptor and glucocorticoid receptor), it is thought that the major coactivator protein holding the steroid hormone receptor-ligand complex in an activated state is erythroblast transformation-specific transcription factor ERG.

Conversely, in respect of the class I steroid hormone receptors (i.e. including estrogen receptor alpha and estrogen receptor beta), it is thought that the major coactivator protein holding the steroid hormone receptor-ligand complex in an activated state is the steroid receptor coactivators, including (e.g.) SRC-1, SRC-2 and/or SRC-3.

It follows that the specificity of the test kits and assays described herein may be enhanced by including at least one steroid hormone receptor corepressor and at least one steroid hormone receptor coactivator. In a related example, the test kits and assays described herein comprise at least one of (i) heat shock protein 90 (HSP90); a complex of HSP90 and heat shock protein 70 (HSP70); a complex of HSP90, HSP70 and heat shock protein 40 (HSP40); a complex of HSP90, HSP70, HSP40 and p23; a complex of HSP90, HSP70, HSP40, p23 and heat shock protein organizing protein (Hop); a complex of HSP90, HSP70, HSP40, p23, Hop and 48 kD Hip protein (Hip); a complex of HSP90, HSP70, HSP40, p23, Hop, Hip and p60; a complex of HSP90, HSP70, HSP40, p23, Hop, Hip, p60 and FKBP52; and any combination thereof, in combination with (ii) at least one of erythroblast transformation-specific transcription factor ERG or at least one steroid receptor coactivator, including but not limited to SRC-1, SRC-2 and/or SRC-3.

It will, however, be appreciated by a person skilled in the art that the presence of at least one steroid hormone regulator protein or molecule is not an essential feature of the test kits, assays and methods described herein. This is because an assay result may still be achieved in the absence of one or both of a corepressor protein or molecule or a coactivator protein or molecule. Hence use of the term “optionally” to define the test kits and assay methods described and claimed herein.

As such, in another aspect of the present invention there is provided a test kit for screening a sample for the presence of a ligand capable of eliciting a steroid hormone genomic response, the test kit comprising:
(i) a cell lysate comprising a steroid hormone receptor that is capable of forming a ligand-receptor complex with a ligand from the sample; and
(ii) at least one steroid hormone receptor coactivator; and/or
(iii) at least one steroid hormone receptor corepressor;
(iv) a nucleic acid molecule comprising:
   (a) a RNA polymerase promoter sequence;
   (b) a response element that is capable of being bound by the ligand receptor complex;
   (c) a reporter construct; and
   (d) optionally, at least one binding site that is capable of being bound by at least one coactivator protein, ERG
   wherein the response element (b) is located between the promoter sequence (a) and the reporter construct (c),
   and wherein the binding site (d) is located immediately upstream (d5′), immediately downstream (d3′), or both immediately upstream (d5′) and immediately downstream (d3′) of the response element (b),
   and wherein the nucleic acid molecule optionally comprises a spacer sequence (e) located between the polymerase promoter sequence (a) and the response element (b)
   provided that [(a), (b) and (c)], [(a), (e), (b) and (c)], [(a), (d5′), (b) and (c)], [(a), (b), (d3′) and (c)], [(a), (e), (d5′), (b) and (c)], [(a), (e), (b), (d3′) and (c)] or [(a), (e), (d5′), (b), (d3′) and (c)] are operably linked; and
(v) optionally, a RNA polymerase.

A person skilled in the art will appreciate that RNA polymerase, including a T7 RNA polymerase, is optionally present in the test kits described herein because it is entirely possible for the enzyme to be sourced externally (e.g. from a laboratory freezer stock).

In an example according to these and other aspects of the present invention, the test kits further comprise nucleotide triphosphates (NTPs).

Also contemplated by the present invention is methods for the detection of steroid hormone ligands by measuring a reduction or inhibition in transcription using the frameworks described above and defined elsewhere in this specification.

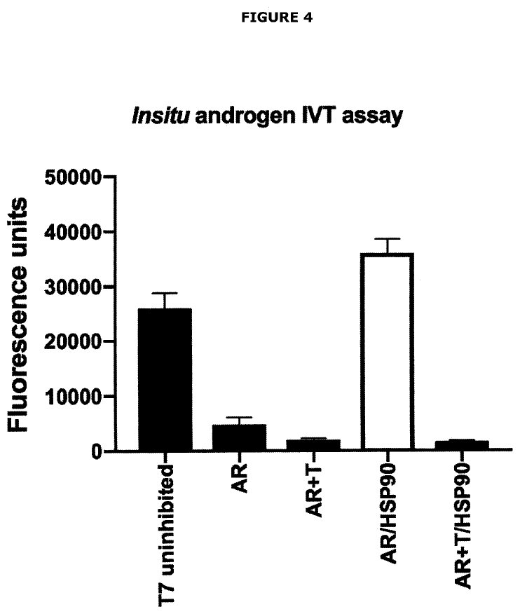

It will, however, be appreciated by a person skilled in the art that the presence of at least one steroid hormone receptor coregulator (e.g. a coactivator and/or a corepressor) is not an essential feature of the test kits, assays and methods described herein. This is because an assay result may still be achieved in the absence of coregulator. For example, the data presented in Example 1/FIG. 4 illustrates there was still a measurable difference in signal between the ligand and non-ligand assay result (i.e. AR/T versus AR) in the absence of heat shock protein 90.

Indeed, there are further approaches in which to minimise the amount of signal generated by auto-activation of the hormone response element by non-liganded receptor, for example, by modifying the temperature at which an assay method is performed.

Applicants have observed a differential in the binding affinty/kinetics between ligand bound and non-ligand bound steroid hormone receptor for its nucleic acid response element. Accordingly, the test kits, assays and methods described herein may be performed at a temperature, or in a temperature range, that preferentially measures activation of a hormone response element by ligand-bound receptor over non-ligand bound receptor, thereby minimising any background signal generated by non-ligand bound receptor.

Accordingly, in yet another example according to this aspect of the present invention, performance of the test kit or assay method is carried out in a temperature range from about 25° C. to about 60° C., and preferably from about 35° C. to about 37° C.

The term “a temperature range from about 25° C. to about 60° C.” is intended to include any temperature from 25° C. to 42° C. and without limitation includes 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C. and 60° C. The skilled person would recognise that temperatures in the decimal point range may also be used. To further illustrate this point, “in a temperature range from about 35° C. to about 37° C.” includes, without limitation, 35.0° C., 35.1° C., 35.2° C., 35.3° C., 35.4° C., 35.5° C., 35.6° C., 35.7° C., 35.8° C., 35.9° C., 36.0° C., 36.1° C., 36.2° C., 36.3° C., 36.4° C., 36.5° C., 36.6° C., 36.7° C., 36.8° C., 36.9° C. and 37.0° C.

The Applicants further discovered that the stoichiometric relationship between the coregulator and steroid hormone receptor may be manipulated to further enhance assay sensitivity. For example, according to the Androgen Assay Prototype 2 described in Example 2, it was determined by the Applicants that AR is most effective at being activated by an AR-specific ligand and binding to ARE when the HSP90:AR ratio is between 1.22 and 4.88.

As previously discussed, in the cell lysate assays described herein, the absolute concentration of receptor remains unknown. However, according to the data presented in Example 7, an optimal HSP90:AR ratio of ˜2:1 was empirically derived based on a AR cell lysate concentration of >30 ng/μL. Refer to FIG. 19 and Table 12. Accordingly, at least for the HEK cell lysates described herein, an optimal ratio of ˜2:1 HSP90:AR is achieved when a concentration of between about 30 ng/μL and about 50 ng/μL of cell lysate is used in the screening assays.

Accordingly, in a further example according to the test kits and assay methods described herein, the ratio of HSP90 to steroid hormone receptor is defined as between about 1:1 to about 5:1. This includes, without limitation, a ratio of HSP90 to steroid hormone receptor that is defined as 1.0:1, 1.1:1, 1.2:1, 1.3:1, 1.4:1, 1.5;1, 1.6:1, 1.7:1, 1.8:1, 1.9:1, 2.0:1, 2.1:1, 2.2:1, 2.3:1, 2.4:1, 2.5;1, 2.6:1, 2.7:1, 2.8:1, 2.9:1, 3.0:1, 3.1:1, 3.2:1, 3.3:1, 3.4:1, 3.5;1, 3.6:1, 3.7:1, 3.8:1, 3.9:1, 4.0:1, 4.1:1, 4.2:1, 4.3:1, 4.4:1, 4.5;1, 4.6:1, 4.7:1, 4.8:1, 4.9:1 or 5.0:1.

The skilled person would, however, appreciate that the molecular stoichiometry between the at least one steroid hormone receptor coregulator and steroid hormone receptor may vary depending on the composition of the test kit/assay. For example, the test kit/assay may be configured to detect ligands which bind to an estrogen receptor, including estrogen receptor alpha or estrogen receptor beta, and the ratio between the estrogen receptor coregulator and estrogen receptor may be (e.g.) between about 1:1 and about 20:1.

Accordingly, in yet another example according to the test kits and assay methods described herein:
(i) the test kit comprises an androgen receptor, and the ratio of androgen receptor coregulator to androgen receptor is between about 1:1 and about 20:1, including without limitation 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1 or 20:1;
(ii) the test kit comprises an estrogen receptor, and the ratio of estrogen receptor coregulator to estrogen receptor is between about 1:1 and about 20:1, including without limitation 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1 or 20:1;
(iii) the test kit comprises a progesterone receptor, and the ratio of progsterone receptor coregulator to progesterone receptor is between about 1:1 and about 20:1, including without limitation 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1 or 20:1;
(iv) the test kit comprises a mineralocorticoid receptor, and the ratio of mineralocorticoid receptor coregulator to mineralocorticoid receptor is between about 1:1 and about 20:1, including without limitation 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1 or 20:1; and
(v) the test kit comprises a glucocorticoid receptor, and the ratio of glucocorticoid receptor coregulator to glucocorticoid receptor is between about 1:1 and about 20:1, including without limitation 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1 or 20:1.

The information in Table 9 of Example 4 summarises key considerations related to the molecular stoichiometry argument when considering the composition of the test kits and assay methods described herein, which is supported by the data accompanying Examples 1-3, in particular.

An assay reaction mix according to the present invention will typically contain between 10 μL and 50 μL total volume. According to the information summarised in Table 9, this means:
(i) the concentration of steroid hormone receptor should be held within a range defined by about 10 nM to about 60 nM which includes, but is not limited to, 10, 11, 12, 13, 14, 15, 16, 17, 18, 19, 20, 21, 22, 23, 24, 25, 26, 27, 28, 29, 30, 31, 32, 33, 34, 35, 36, 37 38, 39, 40, 41, 42, 43, 44, 45, 46, 47, 48, 49, 50, 51, 52, 53, 54, 55, 56, 57, 58, 59 or 60 nM of steroid hormone receptor;
(ii) the concentration of nucleic acid molecule comprising a hormone response element should be held in a range defined by about 0.70 nM to about 3.5 nM which includes, but is not limited to, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2 or 3.3 nM of nucleic acid molecule; and
(iii) where present, the concentration of heat shock protein 90 should be held in a range defined by about 40 nM to about 60 nM which includes, but is not limited to, 40, 41, 42, 43, 44, 45, 46, 47, 48, 49, 50, 51, 52, 53, 54, 55, 56, 57, 58, 59 or 60 nM of heat shock protein 90.

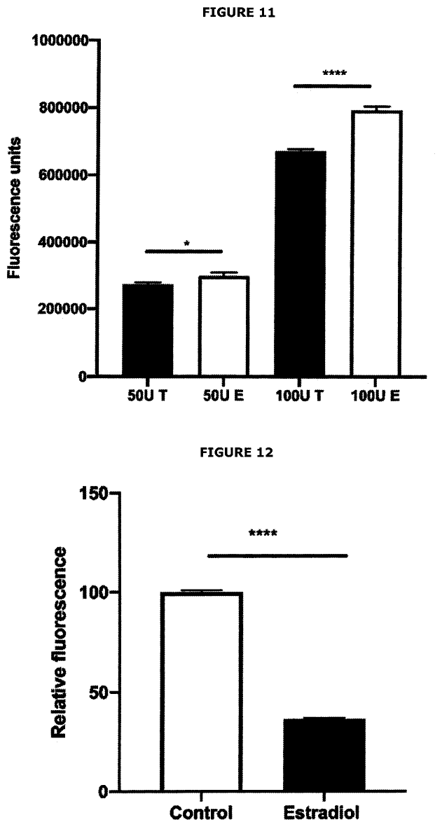

A further consideration is the concentration/amount of enzyme, where optimal assay results were achieved when −50-100 enzyme units (U) were employed in the reaction mixes assuming an absolute baseline threshold activity of about 200,000 U (when RNA aptamer Mango II is the readout by example). Refer to FIGS. 10 and 11.

In yet another example according to the test kits and assay methods described herein, the reduction or inhibition in transcription is a reduction or inhibition in transcription mediated by a single polypeptide polymerase selected from a bacteriophage RNA polymerase, a virus RNA polymerase, a bacterial RNA polymerase, and a eukaryotic virus RNA polymerase.

In a related example, the polymerase is a bacteriophage RNA polymerase. In a further related example, the promoter sequence is a bacteriophage RNA polymerase initiation sequence.

In yet another example according to the test kits and assay methods described herein, the polymerase is a bacteriophage polymerase selected from the group consisting of DNA-dependent RNA polymerases including T7 RNA polymerase, T3 RNA polymerase and SP6 RNA polymerase.

In another example according to the test kits and assay methods described herein, the bacteriophage polymerase is T7 RNA polymerase.

In an example according to this aspect of the present invention, the test kit further comprises nucleotide triphosphates (NTPs).

In another example according to the test kits and assay methods described herein, the promoter sequence is a T7 RNA polymerase initiation sequence.

In a related example, the T7 RNA polymerase initiation sequence comprises a sequence defined by 5′-TAATACGACTCACTATAG-3′ (SEQ ID NO: 82).

Applicants next investigated the T7 promoter architecture in an attempt to further optimize the sensitivity of the steroid hormone screnning assays.

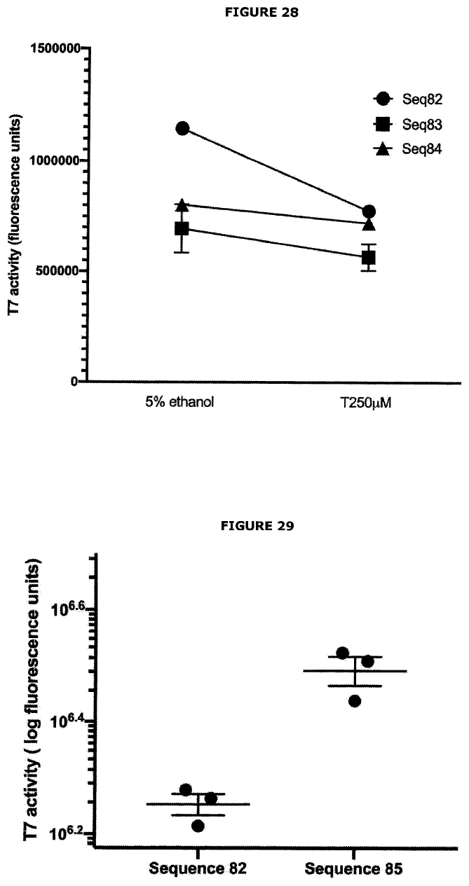

The literature describes various T7 promoter (or initiation) sequences wherein alterations in the nucleotide composition yield increased T7 activity. For example the wild-type T7 promoter sequence defined by 5′-TAATACGACTCACTATAGGGAGA-3′ (SEQ ID NO: 83) has been modified at its 3′ end to yield 5′-TAATACGACTCACAATCGCGGAG-3′ (SEQ ID NO: 84). This variant promoter reportedly facilitates a ˜2-fold increase in T7 activity. Indeed, this was confirmed by the T7 screening assay experiments described in Example 9 and read in conjunction with FIGS. 27 and 28. These data compare the relative activity of these promoters in a T7 screening assay against SEQ ID NO: 82, being a truncated version of the wild-type promoter (i.e. SEQ ID NO: 83) which is widely reported in the literature. There was a ˜2-fold increase in activity observed for SEQ ID NO: 84 over SEQ ID NO: 83, although SEQ ID NO: 82 was still the best performing promoter sequence.

Accordingly, in a related example, the T7 RNA polymerase initiation sequence comprises a sequence defined by 5′-TAATACGACTCACAATCGCGGAG-3′ (SEQ ID NO: 84).

However, Applicants discovered that a modified form of the T7 promoter, containing both 3′ and 5′ modifications and having a sequence defined by 5′-GGAGGCCGGAGAATTGTAATACGACTCACTATAGGGAGACGCGTGT-3′ (SEQ ID NO: 85) yielded superior performance in terms of enhanced sensitivity of the T7 screening assay for detection of androgenic ligands. Refer to Example 9, read in conjunction with FIGS. 29-31, where SEQ ID NO: 85 out-performed SEQ ID NO: 82 in terms of (relative) T7 activity and androgen responsiveness.

As such, in a related example, the T7 RNA polymerase initiation sequence comprises a sequence defined by 5′-GGAGGCCGGAGAATTGTAATACGACTCACTATAGGGAGACGCGTGT-3′ (SEQ ID NO: 85).

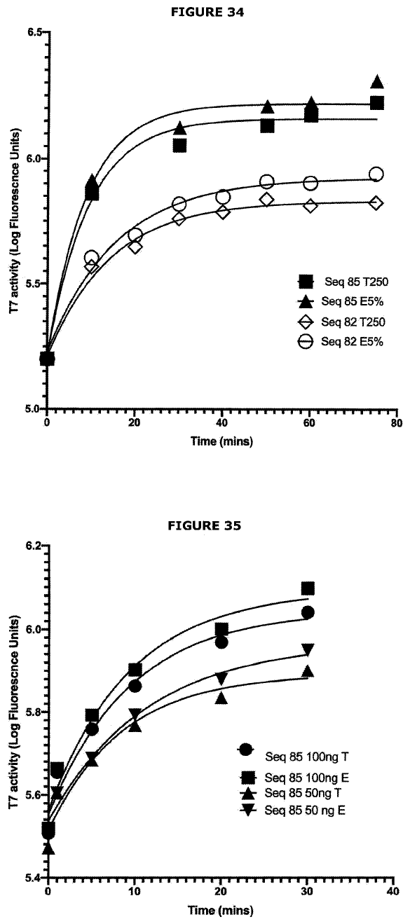

The data presented in FIG. 33, when read in conjunction with the data presented in FIGS. 34-36, further reinforce the utility of cell lysate as a source of steroid hormone receptor for the various T7 screening assays described herein. In particular, and with reference to Example 9 which details the further experimental work undertaken to optimise the relationship(s) between key assay components, these data demonstrate that a T7 screening assay comprising a T7 promoter sequence defined by SEQ ID NO: 85 allows for a faster, more reproducible test. Indeed, Applicants show that the reaction time may be reduced from 150 mins to 40 mins, with greater observed change from vehicle controls. Collectively, these data demonstrate that the cell lysate screening assays described herein are well suited for field applications than would otherwise be expected.

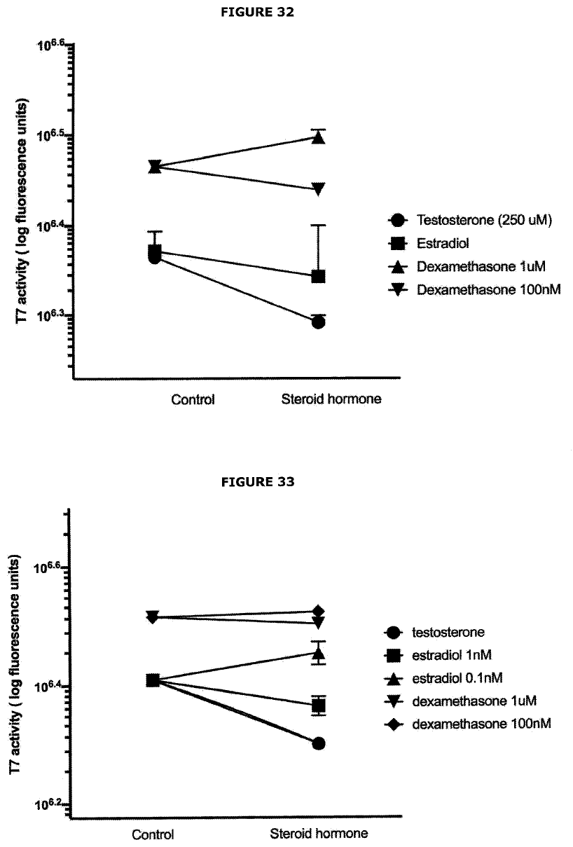

Importantly, the T7 promoter/initiation sequence defined by SEQ ID NO: 85 did not affect performance of the screening assay in terms of cross-activation by E2 or dexamethasone. This observation held true whether a recombinant form of androgen receptor was used (i.e. FIG. 32) or an androgen receptor cell lysate (i.e. FIG. 33).

Accordingly, in another aspect of the present invention there is provided a test kit for screening a sample for the presence of a ligand capable of eliciting a steroid hormone genomic response, the test kit comprising:
(i) a steroid hormone receptor or a cell lysate comprising a steroid hormone receptor, wherein the steroid hormone receptor is capable of forming a ligand-receptor complex with a ligand from the sample; and
(ii) a nucleic acid molecule comprising:
   (a) a T7 RNA polymerase promoter sequence comprising or consisting in a sequence defined by SEQ ID NO: 85;
   (b) a response element that is capable of being bound by the ligand receptor complex; and
   (c) a reporter construct
   wherein the response element (b) is located between the promoter sequence (a) and the reporter construct (c),
   and wherein the nucleic acid molecule optionally comprises a spacer sequence (e) located between the polymerase promoter sequence (a) and the response element (b),
   provided that [(a), (b) and (c)] or [(a), (e), (b) and (c)] are operably linked; and
(iii) optionally, a T7 RNA polymerase.

In an example according to this aspect of the present invention, the test kit further comprises nucleotide triphosphates (NTPs).

Also contemplated by the present invention is methods for the detection of steroid hormone ligands by measuring a reduction or inhibition in transcription using the framework described above.

Accordingly, in yet another aspect of the present invention there is provided an assay method for detecting a ligand in a sample which ligand is capable of eliciting a steroid hormone genomic response, the assay method comprising the steps of:
(1) contacting a sample with:
   (i) a steroid hormone receptor or a cell lysate comprising a steroid hormone receptor, wherein the steroid hormone receptor forms a ligand-receptor complex with a ligand from the sample; and
   (ii) a nucleic acid molecule comprising:
      (a) a T7 RNA polymerase promoter sequence comprising or consisting in a sequence defined by SEQ ID NO: 85;
      (b) a response element that is bound by the ligand-receptor complex; and
      (c) a reporter construct
      wherein the response element (b) is located between the promoter sequence (a) and the reporter construct (c),
      and wherein the nucleic acid molecule optionally comprises a spacer sequence (e) located between the polymerase promoter sequence (a) and the response element (b),
      provided that [(a), (b) and (c)] or [(a), (e), (b) and (c)] are operably linked; and
   (iii) a T7 RNA polymerase; and
   (iv) ribonucleoside triphosphates; and
(2) measuring a reduction or inhibition in transcription of the reporter construct caused by binding of the ligand-receptor complex to the response element,
wherein, a measured reduction or inhibition in transcription of the reporter construct reflects detection of a ligand in the sample.

While not an essential feature of the screening assays described herein, the inclusion of the steroid hormone coregulators as defined herein is further contemplated for these and other aspects of the present invention.

Accordingly, in yet another aspect of the present invention there is provided a test kit for screening a sample for the presence of a ligand capable of eliciting a steroid hormone genomic response, the test kit comprising:
(i) a steroid hormone receptor or a cell lysate comprising a steroid hormone receptor, wherein the steroid hormone receptor is capable of forming a ligand-receptor complex with a ligand from the sample; and
(ii) at least one steroid hormone receptor coactivator; and/or
(iii) at least one steroid hormone receptor corepressor;
(iv) a nucleic acid molecule comprising:
   (a) a T7 RNA polymerase promoter sequence comprising or consisting in SEQ ID NO: 85;
   (b) a response element that is capable of being bound by the ligand receptor complex;
   (c) a reporter construct; and
   (d) optionally, at least one binding site that is capable of being bound by at least one coactivator protein, ERG
   wherein the response element (b) is located between the promoter sequence (a) and the reporter construct (c),
   and wherein the binding site (d) is located immediately upstream (d5′), immediately downstream (d3′), or both immediately upstream (d5′) and immediately downstream (d3′) of the response element (b),
   and wherein the nucleic acid molecule optionally comprises a spacer sequence (e) located between the polymerase promoter sequence (a) and the response element (b)
   provided that [(a), (b) and (c)], [(a), (e), (b) and (c)], [(a), (d5′), (b) and (c)], [(a), (b), (d3′) and (c)], [(a), (e), (d5′), (b) and (c)], [(a), (e), (b), (d3′) and (c)] or [(a), (e), (d5′), (b), (d3′) and (c)] are operably linked; and
(v) optionally, a T7 RNA polymerase.

In an example according to this aspect of the present invention, the test kit further comprises nucleotide triphosphates (NTPs).

Also contemplated by the present invention is methods for the detection of steroid hormone ligands by measuring a reduction or inhibition in transcription using the framework described above and defined elsewhere in this specification.

In a further example according to the test kits and assay methods described herein, the bacteriophage polymerase is T3 RNA polymerase and the promoter sequence is a T3 RNA polymerase initiation sequence.

In a related example, the T3 RNA polymerase initiation sequence comprises a sequence defined by 5′-AATTAACCCTCACTAAAG-3′ (SEQ ID NO: 2).

In yet a further example according to the test kits and assay methods described herein, the bacteriophage polymerase is SP6 RNA polymerase and the promoter sequence is a SP6 RNA polymerase initiation sequence.

In a related example, the SP6 RNA polymerase initiation sequence comprises a sequence defined by 5′-ATTTAGGTGACACTATAG-3′ (SEQ ID NO: 3).

In yet another example according to the test kits and assay methods described herein, the reporter construct comprises a sequence encoding an RNA aptamer that when transcribed to form an RNA aptamer is capable of binding to a fluorophore thereby generating a fluorescence signal.

In a related example, the RNA aptamer is further supported by an RNA scaffold which promotes secondary structure formation of the RNA aptamer, thereby optimizing the binding interaction with its fluorophore(s). In a further related example, the RNA scaffold includes, but is not limited to, F30.

In another example, the RNA aptamer is Mango including, but not limited to, Mango I, Mango II, Mango III and Mango IV. In a related example, the fluorophore which binds to the Mango RNA aptamer thereby generating a fluorescent signal is a derivative of thiazole orange (TO).

In another example, the RNA aptamer is selected from Spinach, iSpinach, baby Spinach and Broccoli. In a related example, the fluorophore which binds to the iSpinach, Spinach or Broccoli RNA aptamer thereby generating a fluorescent signal is 3,5-difluoro-4-hydroxybenzylidene imidazolinone (DFHBI).

In another example, the RNA aptamer is Malachite Green. In a related example, the fluorophore which binds to the Malachite Green RNA aptamer thereby generating a fluorescent signal is malachite green.

In a further example according to the test kits and assay methods described herein, the reporter construct comprises a single sequence copy of the RNA aptamer, or multiple sequence copies of the RNA aptamer. The term “multiple sequence copies” is intended to mean, without limitation, two, three, four, five, six, seven, eight, nine, 10, 11, 12, 13, 14, 15, 16, 17, 18, 19, 20, 21, 22, 23, 24, 25, 26, 27, 28, 29, 30, 31, 32 or more copies of the sequence encoding the RNA aptamer. A person skilled in the art will recognize that the copy number of RNA aptamer sequences will be governed by the optimal signal to noise ratio, as determined by routine assay optimization.

In other examples according to the test kits and assay methods described herein, the reporter construct is selected from the group consisting of a gene that does or does not encode a protein or polypeptide that can be detected by RNA analysis.

As such, the test kits and assay methods described herein may be configured to detect transcript levels of a reporter construct by investigating, for example, messenger ribonucleic acid (mRNA) or complementary deoxyribose nucleic acid (cDNA) levels when a test sample is combined with the assay, as a means to screen the sample for the presence of a ligand having steroid hormone receptor activity.

In yet a further example according to the test kits and assay methods described herein, a reduction or inhibition in transcription of a reporter construct may be measured using polymerase chain reaction (PCR) quantitative PCR (also known as real-time PCR, qPCR), digital PCR (dPCR), reverse transcription PCR (RT-PCR), reverse transcription qPCR (RTqPCR), reverse transcription digital PCR (RTdPCR), RNA seq or insitu hybridisation.

In other examples according to the test kits and assay methods described herein, reporter gene transcript levels comprising (e.g.) mRNA or cDNA may be semi-/quantified using established techniques such as quantitative polymerase chain reaction (qPCR, including RealTime qPCR and Reverse Transcription-qPCR), or using other techniques such as fluorescence based on detection of intercalating dyes or on direct addition of fluorescent nucleotides. For example, the use of fluorophores that bind to RNA aptamers to form RNA-fluorophore complexes, as described herein. These and other techniques would be known to a person skilled in the art.

The test kits and assay methods described herein are configured for detection of various ligands of both known and unknown structure which will bind to a steroid hormone receptor including androgen receptor (AR), estrogen receptor alpha (ER-α), estrogen receptor beta (ER-β), progesterone receptor A (PRA), progesterone receptor B (PRB), mineralocorticoid receptor (MR) and glucocorticoid receptor (GR).

Examples of ligands known to bind androgen receptor include, without limitation, Testosterone, Dihydrotestosterone; Androgenic Anabolic Steroids (AAS) including but not limited to TRENA, 17α-Trenbolone, 17β-Trenbolone, Trendione, Nandrolone, Boldenone, Selective Adrogen Receptor Modulators (SARMs) including but not limited to 93746, BMS-546929, LGD4033, ACP105, YK-11, Andarine, Ligandrol, Ostarine. Androgenic progestagens including but not limited to Altrenogest.

Examples of ligands known to bind estrogen receptor alpha include, without limitation, Estradiol, Estrone, Estriol; Selective Estrogen Receptor Modulators including Raloxifene, Tamoxifen, Toremifene, Ospemifene, Lasofoxifene, Cyclofenil, Clomifene, Broparestrol, Basedoxifene, Anordrin; Phytoestrogens including but not limited to, dietary estrogens such as Polyphenols (Resveratrol), Flavanones (Eriodictyol, Hesperetin, Homoeriodictyol, Naringenin), Flavones (Apigenin, Luteolin, Tangeritin), Flavonols (Fisetin, Kaempferol, Myricetin, Pachypodol, Quercetin, Rhamnazin), Catechins (Proanthocyanides), Isoflavonoids (Isoflavones Biochanin A, Clycitein, Daidzein, Formononetin, Genistein), Isoflavans (Equol), Coumestans (Coumestrol); Estrogen-like Endocrine Disruptive Chemicals (EEDC) including, but not limited to, Dichlorodiphenyltrichloroethane (DDT), Dioxin, Polychlorinated Biphenyls (PCBs), Bisphenol A (BPA), Polybrominated Biphenyls (PBB), Phthalate Esters, Endosulfan, Atrazine, Zeranol; designer compounds such as Hydrazide Derivatives.

Examples of ligands known to bind estrogen receptor beta include, without limitation, all ligands which bind to estrogen receptor alpha, as well as, Diarylpropionitrile (DPN) and Wyeth-derived Benzoxazoles such as Way-659, Way-818 and Way-200070.

Examples of ligands known to bind progesterone receptor A and progesterone receptor B include, without limitation, Progesterone, Norethisterone, Levonorgestrel, Medroxyprogesterone Acetate, Megestrol Acetate, Dydrogesterone, Drospirenone, Altrenogest; Selective Progesterone Receptor Modulators including Ulipristal Acetate, Telapristone Acetate, Vilaprisan, Asoprisnil, Asoprisnil Ecamate; Anti-Progestins including Mifepristone, Onapristone, Lilopritone and Gestrinone.

Examples of ligands known to bind mineralocorticoid receptor include, without limitation, Aldosterone; synthetic mineralocorticoids such as Fludrocortisone; antimineralocorticoids such as Spironolactone and Eplerenone; glucocorticoid receptor ligands such as those described below.

Examples of ligands known to bind glucocorticoid receptor include, without limitation dexamethasone, hydrocortisone, cortisone, prednisolone, methylprednisolone, prednisone, amcinonide, budesonide, desonide, fluocinonide, halcinonide, beclometasone, betamethasone, fluocortolone, halometasone, mometasone, or as antagonists mifepristone, and ketoconazole.

### Exemplary Assay Components & Constructs

Steroid hormone receptors activated by a ligand present in a sample will dimerize (i.e. forming a receptor-ligand complex as defined) and bind to its complimentary hormone response element. In the test kits and assays described herein, hormone response elements are linked to a reporter construct, and a change in a physical property of the reporter construct may be used to reflect the presence of a ligand in a sample under investigation. Exemplary hormone response elements according to the present invention include: androgen response element (ARE), estrogen response element (ERE), progesterone response element (PRE), mineralocorticoid response element (MRE) and glucocorticoid response element (GRE).

As previously stated, the various hormone response elements incorporate binding motifs configured to selectively bind activated ligand-receptor complexes. For example, each of the androgen, estrogen, progesterone, mineralocorticoid and glucocorticoid response elements comprise imperfect dihexameric palindrome sequences which in their secondary structure orientations facilitate binding of a dimerized ligand receptor complex (i.e. (HR-L)₂) via zinc finger binding motifs.

In an example according to the test kits and assay methods described herein, the androgen response element comprises a DNA binding motif that binds to an activated androgen receptor. In a related example, the DNA binding motif binds to a dimer of the ligand bound androgen receptor (i.e. (AR-L)₂; where “AR” is an androgen receptor and “L” is a ligand). In a related example, the DNA binding motif comprises imperfect dihexameric palindrome sequences which create binding specificity between the activated androgen receptor and an androgen response element. In a further related example, the androgen response element comprising a DNA binding motif is a double stranded deoxyribonucleic acid.

In an example according to the test kits and assay methods described herein, the estrogen response element comprises a DNA binding motif that binds to an activated estrogen receptor. In a related example, the DNA binding motif binds to a dimer of the ligand bound estrogen receptor (i.e. (ER-L)₂; where “ER” is an estrogen receptor selected from ER-α or ER-β). In a further related example, the DNA binding motif comprises imperfect dihexameric palindrome sequences which create binding specificity between the activated estrogen receptor and an estrogen response element. In a further related example, the estrogen response element comprising a DNA binding motif is a double stranded deoxyribonucleic acid.

In an example according to the test kits and assay methods described herein, the progesterone response element comprises a DNA binding motif that binds to an activated progesterone receptor. In a related example, the DNA binding motif binds to a dimer of the ligand bound progesterone receptor (i.e. (PR-L)₂; where “PR” is a progesterone receptor selected from PRA or PRB). In a further related example, the DNA binding motif comprises imperfect dihexameric palindrome sequences which create binding specificity between the activated progesterone receptor and a progesterone response element. In a further related example, the progesterone response element comprising a DNA binding motif is a double stranded deoxyribonucleic acid.

In an example according to the test kits and assay methods described herein, the mineralocorticoid response element comprises a DNA binding motif that selectively binds to an activated mineralocorticoid receptor. In a related example, the DNA binding motif binds to a dimer of the ligand bound mineralocorticoid receptor (i.e. (MR-L)₂; where “MR” is a mineralocorticoid receptor). In a further related example, the DNA binding motif comprises imperfect dihexameric palindrome sequences which create binding specificity between the activated mineralocorticoid receptor and a mineralocorticoid response element. In a further related example, the mineralocorticoid response element comprising a DNA binding motif is a double stranded deoxyribonucleic acid.

In an example according to the test kits and assay methods described herein, the glucocorticoid response element comprises a DNA binding motif that selectively binds to an activated glucocorticoid receptor. In a related example, the DNA binding motif binds to a dimer of the ligand bound glucocorticoid receptor (i.e. (GR-L)₂; where “GR” is a glucocorticoid receptor). In a further related example, the DNA binding motif comprises imperfect dihexameric palindrome sequences which create binding specificity between the activated glucocorticoid receptor and a glucocorticoid response element. In a further related example, the glucocorticoid response element comprising a DNA binding motif is a double stranded deoxyribonucleic acid.

Detection of a ligand that binds to and activates an androgen receptor, such as Testosterone, Dihydrotestosterone, androgenic anabolic steroids and selective androgen receptor modulators (i.e SARMs) and phyto- or xenoandrogens, requires test kits/assays comprising an androgen receptor together with an androgen response element capable of binding to an activated androgen-receptor complex.

In an example according to the test kits and assay methods described herein, the androgen response element comprises or consist in the sequence 5′-AGAACAnnnTGTTCT-3′ (SEQ ID NO: 4), where n is any nucleic acid base selected from G, C, T or A. Its complimentary antisense sequence is defined as 5′-AGAACAnnnTGTTCT-3′ (SEQ ID NO: 5), where n represents the base that is complementary to SEQ ID NO: 4 based on a sequence alignment between SEQ ID NOs: 4 and 5 (i.e. A=T; T=A; G=C; C=G).

In another example according to the test kits and assay methods described herein, the androgen response element comprises or consist in the sequence 5′-GGTACAnnnTGTTCT-3′ (SEQ ID NO: 6), where n is any nucleic acid base selected from G, C, T or A. Its complimentary antisense sequence is defined as 5′-AGAACAnnnTGTACC-3′ (SEQ ID NO: 7), where n represents the base that is complementary to SEQ ID NO: 6 based on a sequence alignment between SEQ ID NOs: 6 and 7 (i.e. A=T; T=A; G=C; C=G).

Detection of a ligand that binds to and activates an estrogen receptor, such as Estradiol, Estrone, other estrogen-like steroid hormones including phyto- and xenoestrogens and selective estrogen receptor modulators (SERMs), requires test kits/assays comprising either an estrogen receptor alpha (ER-α) or estrogen receptor beta (ER-β) together with an estrogen response element capable of binding to an activated estrogen-receptor complex.

In an example according to the test kits and assay methods described herein, the estrogen response element comprises or consist in the sequence 5′-AGGTCAnnnTGACCT-3′ (SEQ ID NO: 8), where n is any nucleic acid base selected from G, C, T or A. Its complimentary antisense sequence is defined as 5′-AGGTCAnnnTGACCT-3′ (SEQ ID NO: 9), where n represents the base that is complementary to SEQ ID NO: 8 based on a sequence alignment between SEQ ID NOs: 8 and 9 (i.e. A=T; T=A; G=C; C=G).

Detection of a ligand that binds to and activates a progesterone receptor, such as Progesterone, Norethisterone, Levonorgestrel, other progesterone-like steroid hormones and selective progesterone receptor modulators (SPRMs), requires test kits/assays comprising either an progesterone receptor A (PRA) or progesterone receptor B (PRB) together with an progesterone response element capable of binding to an activated progesterone-receptor complex.

In an example according to the test kits and assay methods described herein, the progesterone response element comprises or consist in the sequence 5′-GGTACAAACTGTTCT-3′ (SEQ ID NO: 10). Its complimentary antisense sequence is defined as 5′-AGAACAGTTTGTACC-3′ (SEQ ID NO: 11).

Detection of a ligand that binds to and activates a mineralocorticoid receptor, such as Aldosterone, synthetic mineralocorticoids such as Fludrocortisone and antimineralocorticoids such as Spironolactone and Eplerenone, requires test kits/assays comprising a mineralocorticoid receptor together with an mineralocorticoid response element capable of binding to an activated mineralocorticoid-receptor complex.

In an example according to the test kits and assay methods described herein, the mineralocorticoid response element comprises or consist in the sequence 5′-AGAACAnAATGTTCT-3′ (SEQ ID NO: 12), where n is any nucleic acid base selected from G, C, T or A. Its complimentary antisense sequence is defined as 5′-AGAACATTnTGTTCT-3′ (SEQ ID NO: 13), where n represents the base that is complementary to SEQ ID NO: 12 based on a sequence alignment between SEQ ID NOs: 12 and 13 (i.e. A=T; T=A; G=C; C=G).

Detection of a ligand that binds to and activates a glucocorticoid receptor, such as, Cortisol, Dexamethasone and 11-Dihydrocorticosterone requires test kits/assays comprising a glucocorticoid receptor together with an glucocorticoid response element capable of binding to an activated glucocorticoid-receptor complex.

In an example according to the test kits and assay methods described herein, the glucocorticoid response element comprises or consist in the sequence 5′-AGAACAnAATGTTCT-3′ (SEQ ID NO: 12), where n is any nucleic acid base selected from G, C, T or A. Its complimentary antisense sequence is defined as 5′-AGAACATTnTGTTCT-3′ (SEQ ID NO: 13), where n represents the base that is complementary to SEQ ID NO: 12 based on a sequence alignment between SEQ ID NOs: 12 and 13 (i.e. A=T; T=A; G=C; C=G).

Exemplary nucleic acid sequences according to the test kits and assay methods described here include, without limitation:
T7i-ARE(n)-MangoII: where n=1, 2, 3, 4;
T7i-ERE(n)-MangoII: where n=1, 2, 3, 4;
T7i-PRE(n)-MangoII: where n=1, 2, 3, 4;
T7i-MRE(n)-MangoII: where n=1, 2, 3, 4;
T7i-GRE(n)-MangoII: where n=1, 2, 3, 4;
T7i-ARE(n)-F30-MangoII: where n=1, 2, 3, 4;
T7i-ERE(n)-F30-MangoII: where n=1, 2, 3, 4;
T7i-PRE(n)-F30-MangoII: where n=1, 2, 3, 4;
T7i-MRE(n)-F30-MangoII: where n=1, 2, 3, 4;
T7i-GRE(n)-F30-MangoII: where n=1, 2, 3, 4;
T7i-ARE(n)-iSpinach: where n=1, 2, 3, 4;
T7i-ERE(n)-iSpinach: where n=1, 2, 3, 4;
T7i-PRE(n)-iSpinach: where n=1, 2, 3, 4;
T7i-MRE(n)-iSpinach: where n=1, 2, 3, 4;
T7i-GRE(n)-iSpinach: where n=1, 2, 3, 4;
T7i-ARE(n)-F30-iSpinach: where n=1, 2, 3, 4;
T7i-ERE(n)-F30-iSpinach: where n=1, 2, 3, 4;
T7i-PRE(n)-F30-iSpinach: where n=1, 2, 3, 4;
T7i-MRE(n)-F30-iSpinach: where n=1, 2, 3, 4;
T7i-GRE(n)-F30-iSpinach: where n=1, 2, 3, 4;
   where T7i represents a promoter/initiation sequence for T7 RNA polymerase.

In a related example, the T7i promoter/initiation sequence is defined by 5′-TAATACGACTCACTATAG-3′ (SEQ ID NO: 1).

In another related example, the T7 RNA polymerase initiation sequence comprises a sequence defined by 5′-TAATACGACTCACTATAGGGAGA-3′ (SEQ ID NO: 83).

In another related example, the T7 RNA polymerase initiation sequence comprises a sequence defined by 5′-TAATACGACTCACAATCGCGGAG-3′ (SEQ ID NO: 84).

In another related example, the T7 RNA polymerase initiation sequence comprises a sequence defined by 5′-GGAGGCCGGAGAATTGTAATACGACTCACTATAGGGAGACGCGTGT-3′ (SEQ ID NO: 85).

Alternate exemplary nucleic acid sequences according to the test kits and assay methods described here include, without limitation:
T3i-ARE(n)-MangoII: where n=1, 2, 3, 4;
T3i-ERE(n)-MangoII: where n=1, 2, 3, 4;
T3i-PRE(n)-MangoII: where n=1, 2, 3, 4;
T3i-MRE(n)-MangoII: where n=1, 2, 3, 4;
T3i-GRE(n)-MangoII: where n=1, 2, 3, 4;
T3i-ARE(n)-F30-MangoII: where n=1, 2, 3, 4;
T3i-ERE(n)-F30-MangoII: where n=1, 2, 3, 4;
T3i-PRE(n)-F30-MangoII: where n=1, 2, 3, 4;
T3i-MRE(n)-F30-MangoII: where n=1, 2, 3, 4;
T3i-GRE(n)-F30-MangoII: where n=1, 2, 3, 4;
T3i-ARE(n)-iSpinach: where n=1, 2, 3, 4;
T3i-ERE(n)-iSpinach: where n=1, 2, 3, 4;
T3i-PRE(n)-iSpinach: where n=1, 2, 3, 4;
T3i-MRE(n)-iSpinach: where n=1, 2, 3, 4;
T3i-GRE(n)-iSpinach: where n=1, 2, 3, 4;
T3i-ARE(n)-iSpinachx3: where n=1, 2, 3, 4;
T3i-ERE(n)-iSpinachx3: where n=1, 2, 3, 4;
T3i-PRE(n)-iSpinachx3: where n=1, 2, 3, 4;
T3i-MRE(n)-iSpinachx3: where n=1, 2, 3, 4;
T3i-GRE(n)-iSpinachx3: where n=1, 2, 3, 4;
where T3i represents a promoter or initiation sequence for T3 RNA polymerase.

In a related example, the T3i promoter/initiation sequence is defined by 5′-AATTAACCCTCACTAAAG-3′ (SEQ ID NO: 2).

Alternate exemplary nucleic acid sequences according to the test kits and assay methods described here include, without limitation:
SP6i-ARE(n)-MangoII: where n=1, 2, 3, 4;
SP6i-ERE(n)-MangoII: where n=1, 2, 3, 4;
SP6i-PRE(n)-MangoII: where n=1, 2, 3, 4;
SP6i-MRE(n)-MangoII: where n=1, 2, 3, 4;
SP6i-GRE(n)-MangoII: where n=1, 2, 3, 4;
SP6i-ARE(n)-F30-MangoII: where n=1, 2, 3, 4;
SP6i-ERE(n)-F30-MangoII: where n=1, 2, 3, 4;
SP6i-PRE(n)-F30-MangoII: where n=1, 2, 3, 4;
SP6i-MRE(n)-F30-MangoII: where n=1, 2, 3, 4;
SP6i-GRE(n)-F30-MangoII: where n=1, 2, 3, 4;
SP6i-ARE(n)-iSpinach: where n=1, 2, 3, 4;
SP6i-ERE(n)-iSpinach: where n=1, 2, 3, 4;
SP6i-PRE(n)-iSpinach: where n=1, 2, 3, 4;
SP6i-MRE(n)-iSpinach: where n=1, 2, 3, 4;
SP6i-GRE(n)-iSpinach: where n=1, 2, 3, 4;
SP6i-ARE(n)-iSpinachx3: where n=1, 2, 3, 4;
SP6i-ERE(n)-iSpinachx3: where n=1, 2, 3, 4;
SP6i-PRE(n)-iSpinachx3: where n=1, 2, 3, 4;
SP6i-MRE(n)-iSpinachx3: where n=1, 2, 3, 4;
SP6i-GRE(n)-iSpinachx3: where n=1, 2, 3, 4;
where SP6i represents a promoter or initiation sequence for SP6 RNA polymerase.

In a related example, the SP6i promoter/initiation sequence is defined by 5′-ATTTAGGTGACACTATAG-3′ (SEQ ID NO: 3).

In another example according to the present invention, the test kits and/or assay methods are configured to detect ligands that bind to an androgen receptor, and the nucleic acid sequence comprised of a T7 RNA polymerase initiation sequence, an androgen response element and a reporter construct encoding F30 scaffold and Mango II RNA aptamer comprises the sequence set forth in SEQ ID NO: 14 as follows:

[SEQ ID NO: 14]
5′GGAGGCCGGAGAATTGTAATACGACTCACTATAGGGAGACGCGTGTA
CTCTGGAGGAATGGAGAACAGCCTGTTCTCCATTGCCATGTGTATGTGG
GTACGAAGGAGAGGAGAGGAAGAGGAGAGTACCCACATACTCTGATGAT
CCTTCGGGATCATTCATGGCAATCTAGGA3′

In yet another example according to the present invention, the test kits and/or assay methods are configured to detect ligands that bind to an estrogen receptor alpha or estrogen receptor beta, and the nucleic acid sequence comprised of a T7 RNA polymerase initiation sequence, an estrogen response element and a reporter construct encoding F30 scaffold and Mango II RNA aptamer comprises the sequence set forth in SEQ ID NO: 15 as follows:

[SEQ ID NO: 15]
5′GGAGGCCGGAGAATTGTAATACGACTCACTATAGGGAGACGCGTGTA
CTCTGGAGGAACAGGTCAGCATGACCTGTTGCCATGTGTATGTGGGTAC
GAAGGAGAGGAGAGGAAGAGGAGAGTACCCACATACTCTGATGATCCTT
CGGGATCATTCATGGCAATCTAGGA3′

In yet a further another example according to the present invention, the test kits and/or assay methods are configured to detect ligands that bind to a progesterone receptor A or a progesterone receptor B, and the nucleic acid sequence comprised of a T7 RNA polymerase initiation sequence, an progesterone response element and a reporter construct encoding F30 scaffold and Mango II RNA aptamer comprises the sequence set forth in SEQ ID NO: 16 as follows:

[SEQ ID NO: 16]
5′GGAGGCCGGAGAATTGTAATACGACTCACTATAGGGAGACGCGTGTA
CTCTGGAGGAAGGTACAAACTGTTCTTTGCCATGTGTATGTGGGTACGA
AGGAGAGGAGAGGAAGAGGAGAGTACCCACATACTCTGATGATCCTTCG
GGATCATTCATGGCAATCTAGGA3′

In another example according to the present invention, the test kits and/or assay methods are configured to detect ligands that bind to a mineralocorticoid receptor, and the nucleic acid sequence comprised of a T7 RNA polymerase initiation sequence, an mineralocorticoid response element and a reporter construct encoding F30 scaffold and Mango II RNA aptamer comprises the sequence set forth in SEQ ID NO: 17 as follows:

[SEQ ID NO: 17]
5′GGAGGCCGGAGAATTGTAATACGACTCACTATAGGGAGACGCGTGTA
CTCTGGAGGAATGTACAGGATGTTCTTTGCCATGTGTATGTGGGTACGA
AGGAGAGGAGAGGAAGAGGAGAGTACCCACATACTCTGATGATCCTTCG
GGATCATTCATGGCAATCTAGGA3′

In another example according to the present invention, the test kits and/or assay methods are configured to detect ligands that bind to a glucocorticoid receptor, and the nucleic acid sequence comprised of a T7 RNA polymerase initiation sequence, an glucocorticoid response element and a reporter construct encoding F30 scaffold and Mango II RNA aptamer comprises the sequence set forth in SEQ ID NO: 18 as follows:

[SEQ ID NO: 18]
5′GGAGGCCGGAGAATTGTAATACGACTCACTATAGGGAGACGCGTGTA
CTCTGGAGGAATGTACAGGATGTTCTTTGCCATGTGTATGTGGGTACGA
AGGAGAGGAGAGGAAGAGGAGAGTACCCACATACTCTGATGATCCTTCG
GGATCATTCATGGCAATCTAGGA3′

Other nucleic acids/constructs for use in the test kits and assay methods according to the present invention are summarised in Table 1, below.

TABLE 1
Exemplary Construct Sequence Information

| | | SEQUENCE |
|---|---|---|
| CONSTRUCT | SEQUENCE | ID NO: |
| T7i-ARE- | GGAGGCCGGAGAATTGTAATACGACTCACTATAGGGAGACGCGTGTA | 19 |
| MangoII | CTCTGGAGGAATGGAGAACAGCCTGTTCTCCATACGAAGGAGAGGAG | |
| | AGGAAGAGGAGAGTA | |
| T7i-ERE- | GGAGGCCGGAGAATTGTAATACGACTCACTATAGGGAGACGCGTGTA | 20 |
| MangoII | CTCTGGAGGAACAGGTCAGCATGACCTGTACGAAGGAGAGGAGAGG | |
| | AAGAGGAGAGTA | |
| T7i-PRE- | GGAGGCCGGAGAATTGTAATACGACTCACTATAGGGAGACGCGTGTA | 21 |
| MangoII | CTCTGGAGGAAGGTACAAACTGTTCTTACGAAGGAGAGGAGAGGAAG | |
| | AGGAGAGTA | |
| T7i-MRE- | GGAGGCCGGAGAATTGTAATACGACTCACTATAGGGAGACGCGTGTA | 22 |
| MangoII | CTCTGGAGGAATGTACAGGATGTTCTTACGAAGGAGAGGAGAGGAAG | |
| | AGGAGAGTA | |
| T7i-GRE- | GGAGGCCGGAGAATTGTAATACGACTCACTATAGGGAGACGCGTGTA | 23 |
| MangoII | CTCTGGAGGAATGTACAGGATGTTCTTACGAAGGAGAGGAGAGGAAG | |
| | AGGAGAGTA | |
| T7i-ARE- | GGAGGCCGGAGAATTGTAATACGACTCACTATAGGGAGACGCGTGTA | 24 |
| iSpinach | CTCTGGAGGAATGGAGAACAGCCTGTTCTCCAAGGAGTACGGTGAGG | |
| | GTCGGGTCCAGTAGGTACGCCTACTGTTGAGTAGAGTGTGGGCTCCG | |
| | TACTCCC | |
| T7i-ERE- | GGAGGCCGGAGAATTGTAATACGACTCACTATAGGGAGACGCGTGTA | 25 |
| iSpinach | CTCTGGAGGAACAGGTCAGCATGACCTGAGGAGTACGGTGAGGGTC | |
| | GGGTCCAGTAGGTACGCCTACTGTTGAGTAGAGTGTGGGCTCCGTAC | |
| | TCCC | |
| T7i-PRE- | GGAGGCCGGAGAATTGTAATACGACTCACTATAGGGAGACGCGTGTA | 26 |
| iSpinach | CTCTGGAGGAAGGTACAAACTGTTCTAGGAGTACGGTGAGGGTCGGG | |
| | TCCAGTAGGTACGCCTACTGTTGAGTAGAGTGTGGGCTCCGTACTCCC | |
| T7i-MRE- | GGAGGCCGGAGAATTGTAATACGACTCACTATAGGGAGACGCGTGTA | 27 |
| iSpinach | CTCTGGAGGAATGTACAGGATGTTCTAGGAGTACGGTGAGGGTCGGG | |
| | TCCAGTAGGTACGCCTACTGTTGAGTAGAGTGTGGGCTCCGTACTCCC | |
| T7i-GRE- | GGAGGCCGGAGAATTGTAATACGACTCACTATAGGGAGACGCGTGTA | 28 |
| iSpinach | CTCTGGAGGAATGTACAGGATGTTCTAGGAGTACGGTGAGGGTCGGG | |
| | TCCAGTAGGTACGCCTACTGTTGAGTAGAGTGTGGGCTCCGTACTCCC | |
| T7i-ARE- | GGAGGCCGGAGAATTGTAATACGACTCACTATAGGGAGACGCGTGTA | 29 |
| F30iSpinach | CTCTGGAGGAATGGAGAACAGCCTGTTCTCCATTGCCATGTGTATGTG | |
| | GGAGGAGTACGGTGAGGGTCGGGTCCAGTAGGTACGCCTACTGTTGA | |
| | GTAGAGTGTGGGCTCCGTACTCCCCCCACATACTCTGATGATCCTTCG | |
| | GGATCATTCATGGCAATCTAGATCTAGA | |
| T7i-ERE- | GGAGGCCGGAGAATTGTAATACGACTCACTATAGGGAGACGCGTGTA | 30 |
| F30iSpinach | CTCTGGAGGAACAGGTCAGCATGACCTGTTGCCATGTGTATGTGGGA | |
| | GGAGTACGGTGAGGGTCGGGTCCAGTAGGTACGCCTACTGTTGAGTA | |
| | GAGTGTGGGCTCCGTACTCCCCCCACATACTCTGATGATCCTTCGGGA | |
| | TCATTCATGGCAATCTAGATCTAGA | |
| T7i-PRE- | GGAGGCCGGAGAATTGTAATACGACTCACTATAGGGAGACGCGTGTA | 31 |
| F30iSpinach | CTCTGGAGGAAGGTACAAACTGTTCTTTGCCATGTGTATGTGGGAGGA | |
| | GTACGGTGAGGGTCGGGTCCAGTAGGTACGCCTACTGTTGAGTAGAG | |
| | TGTGGGCTCCGTACTCCCCCCACATACTCTGATGATCCTTCGGGATCA | |
| | TTCATGGCAATCTAGATCTAGA | |
| T7i-MRE- | GGAGGCCGGAGAATTGTAATACGACTCACTATAGGGAGACGCGTGTA | 32 |
| F30iSpinach | CTCTGGAGGAATGTACAGGATGTTCTTTGCCATGTGTATGTGGGAGGA | |
| | GTACGGTGAGGGTCGGGTCCAGTAGGTACGCCTACTGTTGAGTAGAG | |
| | TGTGGGCTCCGTACTCCCCCCACATACTCTGATGATCCTTCGGGATCA | |
| | TTCATGGCAATCTAGATCTAGA | |
| T7i-GRE- | GGAGGCCGGAGAATTGTAATACGACTCACTATAGGGAGACGCGTGTA | 33 |
| F30iSpinach | CTCTGGAGGAATGTACAGGATGTTCTTTGCCATGTGTATGTGGGAGGA | |
| | GTACGGTGAGGGTCGGGTCCAGTAGGTACGCCTACTGTTGAGTAGAG | |
| | TGTGGGCTCCGTACTCCCCCCACATACTCTGATGATCCTTCGGGATCA | |
| | TTCATGGCAATCTAGATCTAGA | |
| T3i-ARE- | AATTAACCCTCACTAAAGACTCTGGAGGAATGGAGAACAGCCTGTTCT | 34 |
| MangoII | CCATACGAAGGAGAGGAGAGGAAGAGGAGAGTA | |
| T3i-ERE- | AATTAACCCTCACTAAAGACTCTGGAGGAACAGGTCAGCATGACCTGT | 35 |
| MangoII | ACGAAGGAGAGGAGAGGAAGAGGAGAGTA | |
| T3i-PRE- | AATTAACCCTCACTAAAGACTCTGGAGGAAGGTACAAACTGTTCTTAC | 36 |
| MangoII | GAAGGAGAGGAGAGGAAGAGGAGAGTA | |
| T3i-MRE- | AATTAACCCTCACTAAAGACTCTGGAGGAATGTACAGGATGTTCTTAC | 37 |
| MangoII | GAAGGAGAGGAGAGGAAGAGGAGAGTA | |
| T3i-GRE- | AATTAACCCTCACTAAAGACTCTGGAGGAATGTACAGGATGTTCTTAC | 38 |
| MangoII | GAAGGAGAGGAGAGGAAGAGGAGAGTA | |
| T3i-ARE- | AATTAACCCTCACTAAAGACTCTGGAGGAATGGAGAACAGCCTGTTCT | 39 |
| F30MangoII | CCATTGCCATGTGTATGTGGGTACGAAGGAGAGGAGAGGAAGAGGA | |
| | GAGTACCCACATACTCTGATGATCCTTCGGGATCATTCATGGCAATCT | |
| | AGGA | |
| T3i-ERE- | AATTAACCCTCACTAAAGACTCTGGAGGAACAGGTCAGCATGACCTG | 40 |
| F30MangoII | TTGCCATGTGTATGTGGGTACGAAGGAGAGGAGAGGAAGAGGAGAG | |
| | TACCCACATACTCTGATGATCCTTCGGGATCATTCATGGCAATCTAGG | |
| | A | |
| T3i-PRE- | AATTAACCCTCACTAAAGACTCTGGAGGAAGGTACAAACTGTTCT | 41 |
| F30MangoII | TTGCCATGTGTATGTGGGTACGAAGGAGAGGAGAGGAAGAGGAGAG | |
| | TACCCACATACTCTGATGATCCTTCGGGATCATTCATGGCAATCTAGG | |
| | A | |
| T3i-MRE- | AATTAACCCTCACTAAAGACTCTGGAGGAATGTACAGGATGTTCT | 42 |
| F30MangoII | TTGCCATGTGTATGTGGGTACGAAGGAGAGGAGAGGAAGAGGAGAG | |
| | TACCCACATACTCTGATGATCCTTCGGGATCATTCATGGCAATCTAGG | |
| | A | |
| T3i-GRE- | AATTAACCCTCACTAAAGACTCTGGAGGAATGTACAGGATGTTCT | 43 |
| F30MangoII | TTGCCATGTGTATGTGGGTACGAAGGAGAGGAGAGGAAGAGGAGAG | |
| | TACCCACATACTCTGATGATCCTTCGGGATCATTCATGGCAATCTAGG | |
| | A | |
| T3i-ARE- | AATTAACCCTCACTAAAGACTCTGGAGGAATGGAGAACAGCCTGTTCT | 44 |
| iSpinach | CCAAGGAGTACGGTGAGGGTCGGGTCCAGTAGGTACGCCTACTGTTG | |
| | AGTAGAGTGTGGGCTCCGTACTCCC | |
| T3i-ERE- | AATTAACCCTCACTAAAGACTCTGGAGGAACAGGTCAGCATGACCTGA | 45 |
| iSpinach | GGAGTACGGTGAGGGTCGGGTCCAGTAGGTACGCCTACTGTTGAGTA | |
| | GAGTGTGGGCTCCGTACTCCC | |
| T3i-PRE- | AATTAACCCTCACTAAAGACTCTGGAGGAAGGTACAAACTGTTCTAGG | 46 |
| iSpinach | AGTACGGTGAGGGTCGGGTCCAGTAGGTACGCCTACTGTTGAGTAGA | |
| | GTGTGGGCTCCGTACTCCC | |
| T3i-MRE- | AATTAACCCTCACTAAAGACTCTGGAGGAATGTACAGGATGTTCTAGG | 47 |
| iSpinach | AGTACGGTGAGGGTCGGGTCCAGTAGGTACGCCTACTGTTGAGTAGA | |
| | GTGTGGGCTCCGTACTCCC | |
| T3i-GRE- | AATTAACCCTCACTAAAGACTCTGGAGGAATGTACAGGATGTTCTAGG | 48 |
| iSpinach | AGTACGGTGAGGGTCGGGTCCAGTAGGTACGCCTACTGTTGAGTAGA | |
| | GTGTGGGCTCCGTACTCCC | |
| T3i-ARE- | AATTAACCCTCACTAAAGACTCTGGAGGAATGGAGAACAGCCTGTTCT | 49 |
| F30iSpinach | CCATTGCCATGTGTATGTGGGAGGAGTACGGTGAGGGTCGGGTCCAG | |
| | TAGGTACGCCTACTGTTGAGTAGAGTGTGGGCTCCGTACTCCCCCCAC | |
| | ATACTCTGATGATCCTTCGGGATCATTCATGGCAATCTAGATCTAGA | |
| T3i-ERE- | AATTAACCCTCACTAAAGACTCTGGAGGAACAGGTCAGCATGACCTGT | 50 |
| F30iSpinach | TGCCATGTGTATGTGGGAGGAGTACGGTGAGGGTCGGGTCCAGTAG | |
| | GTACGCCTACTGTTGAGTAGAGTGTGGGCTCCGTACTCCCCCCACATA | |
| | CTCTGATGATCCTTCGGGATCATTCATGGCAATCTAGATCTAGA | |
| T3i-PRE- | AATTAACCCTCACTAAAGACTCTGGAGGAAGGTACAAACTGTTCTTTG | 51 |
| F30iSpinach | CCATGTGTATGTGGGAGGAGTACGGTGAGGGTCGGGTCCAGTAGGTA | |
| | CGCCTACTGTTGAGTAGAGTGTGGGCTCCGTACTCCCCCCACATACTC | |
| | TGATGATCCTTCGGGATCATTCATGGCAATCTAGATCTAGA | |
| T3i-MRE- | AATTAACCCTCACTAAAGACTCTGGAGGAATGTACAGGATGTTCTTTG | 52 |
| F30iSpinach | CCATGTGTATGTGGGAGGAGTACGGTGAGGGTCGGGTCCAGTAGGTA | |
| | CGCCTACTGTTGAGTAGAGTGTGGGCTCCGTACTCCCCCCACATACTC | |
| | TGATGATCCTTCGGGATCATTCATGGCAATCTAGATCTAGA | |
| T3i-GRE- | AATTAACCCTCACTAAAGACTCTGGAGGAATGTACAGGATGTTCTTTG | 53 |
| F30iSpinach | CCATGTGTATGTGGGAGGAGTACGGTGAGGGTCGGGTCCAGTAGGTA | |
| | CGCCTACTGTTGAGTAGAGTGTGGGCTCCGTACTCCCCCCACATACTC | |
| | TGATGATCCTTCGGGATCATTCATGGCAATCTAGATCTAGA | |
| SP6i-ARE- | ATTTAGGTGACACTATAGACTCTGGAGGAATGGAGAACAGCCTGTTCT | 54 |
| MangoII | CCATACGAAGGAGAGGAGAGGAAGAGGAGAGTA | |
| SP6i-ERE- | ATTTAGGTGACACTATAGACTCTGGAGGAACAGGTCAGCATGACCTGT | 55 |
| MangoII | ACGAAGGAGAGGAGAGGAAGAGGAGAGTA | |
| SP6i-PRE- | ATTTAGGTGACACTATAGACTCTGGAGGAAGGTACAAACTGTTCTTAC | 56 |
| MangoII | GAAGGAGAGGAGAGGAAGAGGAGAGTA | |
| SP6i-MRE- | ATTTAGGTGACACTATAGACTCTGGAGGAATGTACAGGATGTTCTTAC | 57 |
| MangoII | GAAGGAGAGGAGAGGAAGAGGAGAGTA | |
| SP6i-GRE- | ATTTAGGTGACACTATAGACTCTGGAGGAATGTACAGGATGTTCTTAC | 58 |
| MangoII | GAAGGAGAGGAGAGGAAGAGGAGAGTA | |
| SP6i-ARE- | ATTTAGGTGACACTATAGACTCTGGAGGAATGGAGAACAGCCTGTTCT | 59 |
| F30MangoII | CCATTGCCATGTGTATGTGGGTACGAAGGAGAGGAGAGGAAGAGGA | |
| | GAGTACCCACATACTCTGATGATCCTTCGGGATCATTCATGGCAATCT | |
| | AGGA | |
| SP6i-ERE- | ATTTAGGTGACACTATAGACTCTGGAGGAACAGGTCAGCATGACCTGT | 60 |
| F30MangoII | TGCCATGTGTATGTGGGTACGAAGGAGAGGAGAGGAAGAGGAGAGT | |
| | ACCCACATACTCTGATGATCCTTCGGGATCATTCATGGCAATCTAGGA | |
| SP6i-PRE- | ATTTAGGTGACACTATAGACTCTGGAGGAAGGTACAAACTGTTCTTTG | 61 |
| F30MangoII | CCATGTGTATGTGGGTACGAAGGAGAGGAGAGGAAGAGGAGAGTAC | |
| | CCACATACTCTGATGATCCTTCGGGATCATTCATGGCAATCTAGGA | |
| SP6i-MRE- | ATTTAGGTGACACTATAGACTCTGGAGGAATGTACAGGATGTTCTTTG | 62 |
| F30MangoII | CCATGTGTATGTGGGTACGAAGGAGAGGAGAGGAAGAGGAGAGTAC | |
| | CCACATACTCTGATGATCCTTCGGGATCATTCATGGCAATCTAGGA | |
| SP6i-GRE- | ATTTAGGTGACACTATAGACTCTGGAGGAATGTACAGGATGTTCTTTG | 63 |
| F30MangoII | CCATGTGTATGTGGGTACGAAGGAGAGGAGAGGAAGAGGAGAGTAC | |
| | CCACATACTCTGATGATCCTTCGGGATCATTCATGGCAATCTAGGA | |
| SP6i-ARE- | ATTTAGGTGACACTATAGACTCTGGAGGAATGGAGAACAGCCTGTTCT | 64 |
| iSpinach | CCAAGGAGTACGGTGAGGGTCGGGTCCAGTAGGTACGCCTACTGTTG | |
| | AGTAGAGTGTGGGCTCCGTACTCCC | |
| SP6i-ERE- | ATTTAGGTGACACTATAGACTCTGGAGGAACAGGTCAGCATGACCTGA | 65 |
| iSpinach | GGAGTACGGTGAGGGTCGGGTCCAGTAGGTACGCCTACTGTTGAGTA | |
| | GAGTGTGGGCTCCGTACTCCC | |
| SP6i-PRE- | ATTTAGGTGACACTATAGACTCTGGAGGAAGGTACAAACTGTTCTAGG | 66 |
| iSpinach | AGTACGGTGAGGGTCGGGTCCAGTAGGTACGCCTACTGTTGAGTAGA | |
| | GTGTGGGCTCCGTACTCCC | |
| SP6i-MRE- | ATTTAGGTGACACTATAGACTCTGGAGGAATGTACAGGATGTTCTAGG | 67 |
| iSpinach | AGTACGGTGAGGGTCGGGTCCAGTAGGTACGCCTACTGTTGAGTAGA | |
| | GTGTGGGCTCCGTACTCCC | |
| SP6i-GRE- | ATTTAGGTGACACTATAGACTCTGGAGGAATGTACAGGATGTTCTAGG | 68 |
| iSpinach | AGTACGGTGAGGGTCGGGTCCAGTAGGTACGCCTACTGTTGAGTAGA | |
| | GTGTGGGCTCCGTACTCCC | |
| SP6i-ARE- | ATTTAGGTGACACTATAGACTCTGGAGGAATGGAGAACAGCCTGTTCT | 69 |
| F30iSpinach | CCATTGCCATGTGTATGTGGGAGGAGTACGGTGAGGGTCGGGTCCAG | |
| | TAGGTACGCCTACTGTTGAGTAGAGTGTGGGCTCCGTACTCCCCCCAC | |
| | ATACTCTGATGATCCTTCGGGATCATTCATGGCAATCTAGATCTAGA | |
| SP6i-ERE- | ATTTAGGTGACACTATAGACTCTGGAGGAACAGGTCAGCATGACCTGT | 70 |
| F30iSpinach | TGCCATGTGTATGTGGGAGGAGTACGGTGAGGGTCGGGTCCAGTAG | |
| | GTACGCCTACTGTTGAGTAGAGTGTGGGCTCCGTACTCCCCCCACATA | |
| | CTCTGATGATCCTTCGGGATCATTCATGGCAATCTAGATCTAGA | |
| SP6i-PRE- | ATTTAGGTGACACTATAGACTCTGGAGGAAGGTACAAACTGTTCTTTG | 71 |
| F30iSpinach | CCATGTGTATGTGGGAGGAGTACGGTGAGGGTCGGGTCCAGTAGGTA | |
| | CGCCTACTGTTGAGTAGAGTGTGGGCTCCGTACTCCCCCCACATACTC | |
| | TGATGATCCTTCGGGATCATTCATGGCAATCTAGATCTAGA | |
| SP6i-MRE- | ATTTAGGTGACACTATAGACTCTGGAGGAATGTACAGGATGTTCTTTG | 72 |
| F30iSpinach | CCATGTGTATGTGGGAGGAGTACGGTGAGGGTCGGGTCCAGTAGGTA | |
| | CGCCTACTGTTGAGTAGAGTGTGGGCTCCGTACTCCCCCCACATACTC | |
| | TGATGATCCTTCGGGATCATTCATGGCAATCTAGATCTAGA | |
| SP6i-GRE- | ATTTAGGTGACACTATAGACTCTGGAGGAATGTACAGGATGTTCTTTG | 73 |
| F30iSpinach | CCATGTGTATGTGGGAGGAGTACGGTGAGGGTCGGGTCCAGTAGGTA | |
| | CGCCTACTGTTGAGTAGAGTGTGGGCTCCGTACTCCCCCCACATACTC | |
| | TGATGATCCTTCGGGATCATTCATGGCAATCTAGATCTAGA | |

### Multiplexed Assay Systems

The present invention further contemplates multiplexed assays configured to detect two or more steroid hormone genomic responses from the same test sample.

To further illustrate the relevance of multiplexed systems, in certain circumstances it would be useful for a clinician investigating, for example, the hormonal status of a subject to know both the androgenic and estrogenic levels/activity in the subject.

The side-by-side detection of androgenic and estrogenic ligands from the same test sample is possible because a ligand which binds to an androgen receptor will not bind to and activate an estrogen receptor present in the same assay; conversely a ligand which binds to an estrogen receptor which will not bind to and activate an androgen receptor also present in the same assay. This is because androgen and estrogen receptors belong to different steroid hormone receptor classes, and so there is no ‘cross-talk’ in terms of receptor activation. And so, multiplexed assays have been developed to detect both androgenic and estrogenic ligands from the same sample.

Accordingly, in another aspect of the present invention there is provided a test kit for screening a sample for the side-by-side detection of an androgenic ligand and/or an estrogenic ligand, the test kit comprising:
(i) a cell lysate comprising an androgen receptor, wherein the androgen receptor is capable of forming an androgen receptor-ligand complex with a complimentary ligand from the sample; and
(ii) a first nucleic acid molecule comprising:
   (a) a polymerase promoter sequence;
   (b) an androgen response element that is capable of being bound by the androgen receptor-ligand complex; and
   (c) a first reporter construct;
   where the response element (b) is located between the promoter sequence (a) and the reporter construct (c), and (a), (b) and (c) are operably linked; and
(iii) the same cell lysate as (i) or a different cell lysate comprising an estrogen receptor, wherein the estrogen receptor is capable of forming an estrogen receptor-ligand complex with a complimentary ligand from the sample; and
(iv) a second nucleic acid molecule comprising:
   (a) the polymerase promoter sequence;
   (b) an estrogen response element that is capable of being bound by the estrogen receptor-ligand complex; and
   (c) a second reporter construct; and
(v) optionally, a single polypeptide polymerase
wherein, the first and second reporter constructs are different.

According to certain examples of the inventions described herein, assay components (i) and (iii) in the aspect referred to immediately above could be provided by the same cell lysate.

According to these and other aspects of the present invention, the test kits may be modified to include least one steroid hormone receptor corepressor and at least one steroid hormone receptor coactivator.

In an example according to this aspect of the present invention, the first and second nucleic acid molecules are discrete molecules.

In another example according to this aspect of the present invention, the first and second nucleic acid molecules are operably linked.

In yet another example according to this aspect of the present invention, the first nucleic acid molecule comprises a sequence defined by SEQ ID NO: 14.

In a further example according to this aspect of the present invention, the second nucleic acid molecule comprises a sequence defined by SEQ ID NO: 15.

In yet a further example according to this aspect of the present invention the first and second nucleic acid molecules are operably linked and comprise a sequence defined by SEQ ID NO: 100.

5′-
GGAGGCCGGAGAATTGTAATACGACTCACTATAGGGAGACGCGTGTACT
CTGGAGGAATGGAGAACAGCCTGTTCTCCATTGCCATGTGTATGTGGGT
ACGAAGGAGAGGAGAGGAAGAGGAGAGTACCCACATACTCTGATGATCC
TTCGGGATCATTCATGGCAATCTAGGAAACCCCGCGGGGCCTTTCGGGG
GTCTCGCGGGGTTTTTTGCTCGGAGGCCGGAGAATTGTAATACGACTCA
CTATAGGGAGACGCGTGTACTCTGGAGGAACAGGTCAGCATGACCTGTT
GCCATGTGTATGTGGGTACGAAGGAGAGGAGAGGAAGAGGAGAGTACCC
ACATACTCTGATGATCCTTCGGGATCATTCATGGCAATCTAGGA-3′

Advantageously, the assays and test kits described herein are particularly suited for configuration in multiplexed systems because the simplicity of the assay systems described herein means that an existing assay or test kit may be routinely modified to include a second or subsequent receptor/report construct combination specific to detection of a second ligand; by leveraging the same in vitro transcription machinary (e.g. bacteriophage polymerase+nucleoside triphosphates), discrete signals generated by each reporter may be conveniently detected. To further illustrate this point, a multiplexed assay system according to the present invention may comprise, for example, an androgen specific reporter construct which, in the presence of androgen or an androgen-like ligand, would generate a reporter read-out that may be measured independently of the read-out generated by a reporter construct that is specific for the detection of an estrogen or an estrogen-like ligand in the same sample.

The skilled person would appreciate the advantages conferred by a lack of molecular complexity associated with the multiplexed systems of the present invention, and would recognise that detection of multiple discrete steroid hormone genomic responses (e.g. two, three, four, or more) from the same test sample is possible.

Accordingly, the test kits according to the present invention comprise at least one steroid hormone receptor and at least one nucleic acid molecule comprising at least one reporter construct.

Accordingly, the term “a steroid hormone receptor” according to the test kits and methods described herein is intended to mean “at least one steroid hormone receptor” in the sense that two or more different types of steroid hormone receptors may be present (e.g. and by way of illustration only, a steroid hormone receptor that binds testosterone and a steroid hormone receptor that binds estradiol).

Similarly, the term “a nucleic acid molecule [comprising a response element]” is intended to mean “at least one nucleic acid” in the sense that two or more discrete nucleic acid molecules may be present, each comprising a different response element and optionally a different reporter molecule.

In an example according to this aspect of the present invention, the test kit comprises (i) an estrogen receptor and nucleic acid molecule comprising an estrogen response element, and (ii) an androgen receptor and nucleic acid molecule comprising an androgen response element.

In a related example, the nucleic acid molecule comprising an estrogen response element further comprises a first RNA aptamer that is capable of binding to a first fluorophore.

In another related example, the nucleic acid molecule comprising the androgen response element further comprises a second RNA aptamer that is capable of binding to a second fluorophore.

In a related example, the first and second RNA aptamers include, but are not limited to Mango I, Mango II, Mango III and Mango IV, Spinach, iSpinach, baby Spinach, Broccoli and Malachite Green, provided the first RNA aptamer is not identical to the second RNA aptamer.

In a further related example, the first RNA aptamer is Mango II and the second RNA aptamer is Malachite Green.

In a further related example, the first RNA aptamer is Mango II and the second RNA aptamer is iSpinach.

### Clinical Applications

The present invention further contemplates a utility for the test kits and assay methods described herein for various clinical applications.

For example, it may desirable to know the total estrogenic activity of a test (e.g. biological) sample. As described elsewhere in this specification, there are two estrogen receptor subtypes, namely estrogen receptor alpha and estrogen receptor beta. Accordingly, the present invention contemplates a multiplexed assay format involving the two estrogen receptor subtypes, based on the assay principles described herein, to measure the total estrogenic activity of a sample.

Accordingly, in yet another aspect of the present invention there is provided a test kit to determine the total estrogenic activity of a test sample, the test kit comprising:
(i) a cell lysate comprising estrogen receptor alpha and estrogen receptor beta;
(ii) optionally, at least one steroid hormone receptor;
(iii) optionally, at least one steroid hormone receptor; and
(iv) a nucleic acid molecule comprising:
   (a) a RNA polymerase promoter sequence;
   (b) a response element that is capable of being bound by the receptor-ligand complex; and
   (c) a reporter construct
   where the response element (b) is located between the promoter sequence (a) and the reporter construct (c), and (a), (b) and (c) are operably linked; and
(v) optionally, a RNA polymerase.

In an example according to this aspect of the present invention, the RNA polymerase is T7 RNA polymerase, and the RNA polymerase promoter sequence is T7 RNA polymerase promoter sequence defined by SEQ ID NO: 85.

In another example according to this aspect of the present invention, the nucleic acid sequence comprises a spacer (e) which is located between the promoter sequence (a) and the response element (b). In a related example, the spacer (e) is between about 2 and about 32 nucleotides in length.

### Utility of the Test Kits & Assays

Advantageously, the present invention provides activity based test kits, assays and methods that work fundamentally on the principle of steroid hormone receptor activation. By detecting steroid hormone receptor activation (with consequent binding to its hormone response element) by a target ligand present within a sample to be tested, the present invention provides cell-free test kits, assays and methods that do not rely on structural knowledge of the ligand(s) being interrogated, can readily distinguish between the presence of biologically active and inactive ligands, and provide cost-effective, reliable and reproducible systems that do not require complex laboratory equipment or particular expertise to perform.

Accordingly, in another aspect of the present invention there is provided a method for determining the doping status of an athlete, the method comprising combining a sample obtained from the athlete with a test kit as described herein and determining the doping status of an athlete.

In an example according to this aspect of the present invention, the sample obtained obtained from the athlete is a serum sample, a plasma sample or a urine sample.

In another example, the athlete is a human athlete or a non-human athlete selected from a horse, a camel or a dog.

In a further aspect of the present invention there is provided an article of manufacture for screening a test sample for the presence of a ligand, which ligand is capable of activating a steroid hormone receptor and eliciting a genomic response in a cell, the article of manufacture comprising a test kit as described herein together with instructions for how to detect the presence of a ligand in the sample.

In yet a further aspect of the present invention there is provided an article of manufacture for determining doping in an athlete, the article of manufacture comprising a test kit as described herein together with instructions for detecting the presence of a ligand in a sample derived from the athlete, wherein the presence of the ligand in the sample is indicative of doping in the athlete.

The various test kits and assays described herein each provide (i) a steroid hormone receptor inclusive of a ligand binding domain for binding a ligand that may be present in a sample to be tested and (ii) a nucleic acid response element comprising a protein binding domain which is bound by an activated steroid hormone receptor (or ligand-receptor complex; HR-L). The term “activated steroid hormone receptor” refers to a receptor-ligand complex, and may include various permutations of the HR-L structure (e.g. monomer, dimer, trimer etc). Importantly, the hormone response element contains binding motifs specific for the receptor-ligand complex. Accordingly, by combining the test kits and assays of the present invention with a sample of interest, detection of a ligand, which possesses the potential to bind to a steroid hormone receptor and elicit a steroid hormone genomic response, is possible.

The terms “receptor binding domain”, “activated receptor binding domain”, “hormone receptor binding domain”, “activated hormone receptor binding domain”, “receptor-ligand binding domain” and “hormone receptor-ligand binding domain” are used interchangeably to refer to the protein binding domain of the hormone response element that is bound by an activated hormone receptor or ligand-receptor complex, as defined herein.

In other examples, the inventions described herein find utility in the detection of performance enhancing pro/drugs (e.g. anabolic steroids) used in human as well as non-human athletes including race horses, camels and dogs. In other examples, the inventions described herein have utility in screening foods and health food supplements for additives that may bind to a steroid hormone receptor and elicit a genomic response in a cell or do so following metabolic processing (i.e. in the case of so-called ‘prodrugs’).

The present invention further contemplates detection of one or more physiologically inactivate ligands from a test sample, which ligands are ultimately capable of activating steroid hormone receptors when converted to a physiologically active form. As such, the test kits, assays and methods as described herein further comprise steroid metabolism machinery that is capable of processing the ligand in such a way that it will activate its corresponding steroid hormone receptor. In this way, detection of physiologically inactive ligands (e.g. prohormones) from samples such as nutritional supplements is possible.

As such, the test kits, assays and methods described herein may further comprise steroid metabolism machinery sufficient to convert a ligand from a physiologically inactive form to a physiologically active form, or from a physiologically active form to a more physiologically active form or from a physiologically active form to a less physiologically active form, or from a physiologically active form to a physiologically inactive form. Only when the ligand is in a physiologically active form does it possess the ability to activate a steroid hormone receptor and elicit a genomic response. Accordingly, inclusion of steroid metabolism machinery in the test kits, assays and methods according to the present invention helps facilitate detection of physiologically inactive ligands from a test sample of interest, (e.g.) which ligands exist as pro-drugs (e.g. pro-hormones) and might otherwise evade detection using established methodologies. Furthermore, inclusion of steroid metabolism machinery in the test kits, assays and methods according to the present invention helps determine biological activity/potency of ligands necessary to show effect.

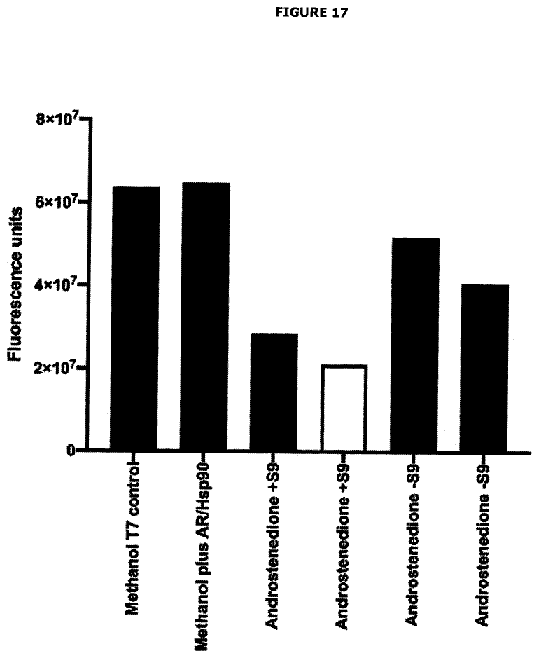

The data presented in FIG. 17 further illustrate this point. Androstenedione (i.e. a androgen prohormone) was preincubated with S9 liver fraction before the reaction mix was extracted for steroids (i.e. to remove any potential non-specific ligands). The various test and control samples were then analysed for androgenic activity. The results demonstrate a statistically significant reduction in fluorescence of the reporter construct for Androstenedione+S9 liver treatment compared to the no-treatment control (i.e. Androstenedione-S9 liver), where the androgenic activity levels for the no treatment control approximated the vehicle control (i.e. methanol+T7; methanol+AR/HSP90).

The test kits, assays and methods described herein may further comprise a detection means for detecting binding between the receptor-ligand complex and the response element contained within the nucleic acid, as defined.

The test kits and assays according to the present invention are cell-free. This is particularly important since the molecular complexity of the assay systems are significantly reduced. For example, the absence of (i) a cell membrane structure which has the potential to create a thermodynamic sink for steroid hormone molecules and (ii) endogenous steroid hormone metabolism observed with cell based systems, provides for an assay system with enhanced sensitivity and selectivity. Further, and advantageously, according to the test kits, assays and methods described herein, the relative amounts of essential structural elements (e.g. steroid hormone receptor and nucleic acid response element inclusive of one or more activated receptor binding domains) may be precisely controlled to provide enhanced assay functionality and increased sensitivity.

According to the methods described herein, the test result may be compared to a reference threshold in order to determine the absolute level of signal generated by a ligand present in a test sample. Indeed, Applicants observed non-specific binding and/or activation of the response element by non-ligand bound receptor. Accordingly, where it is desirable to perform a semi-quantitative analysis for any given test sample, the assays and methods described herein may be performed in the absence of test sample to first establish a reference threshold (e.g. in presence of ethanol acting as a negative control). Assay results obtained from a test sample may then be compared to the reference threshold, to determine the absolute activity attributable to the ligand(s) present in a sample using a simple subtraction methodology.

The present invention further contemplates the use of the assays and test kits described herein to determine the potency of a test compound relative to a reference compound. According to the present invention, the term ‘relative potency’ is defined as the multiplier of biological activity of a test compound relative to a reference compound, as determined by normalizing the biological activity of the test compound to the reference compound.

The biological activity of the test and reference compounds may be determined using EC₅₀ or the concentration of compound that gives half the maximal response from a dose response curve for that particular compound. The dose response curve is generated by serially diluting the compound and measuring its steroid hormone receptor binding/activation profile. A plot of the measured activity (e.g. as measured by fluorescence) vs concentration of the compound (i.e. serial dilution of the compound generates a concentration range that is best presented on a log scale) is then made.

A person skilled in the art will recognize that a measure of relative potency of a test compound is relative to the reference compound used. In other words, the relative potency of a test compound is likely to differ depending on the reference compound to which its biological activity is normalized.

Where the relative potency is >1, the test compound invokes a higher measured biological activity in the assays compared to the reference compound. Where the relative potency is <1, the test compound invokes a lower measured biological activity in the assays compared to the reference compound. Where the relative potency=1, the test compound and the reference compound invoke equal biological activity in the assays.

Relative potency can also be used to determine the activation factor of a test compound in question. As used herein, activation factor relates the relative potency of a test compound determined in a yeast cell or using a yeast cell-free extract (i.e. which contains no metabolic machinery) to the relative potency of the same test compound determined in a mammalian cell or using a mammalian cell-free extract (i.e. which includes metabolic machinery) as a measure of relative activation between the two states of the test compound. An activation factor >1 means that the test compound has undergone metabolic conversion to a more physiologically active state in the presence of metabolic machinery in the assay.

Yet another advantage conferred by the test kits and assays according to the present invention is the relative ease of performance. In other words, performance of the test kits, assays and methods described herein does not require complex cell culture techniques, experienced laboratory technicians or convoluted laboratory testing equipment and analysis. This is particularly advantageous, because the test kits, assays and methods according to the present invention may be practiced by untrained personnel in the field following relatively simple testing procedures. Further, performance of the test kits, assays and methods may provide real time information (e.g.) when testing for performance enhancing substances in a sample taken from an athlete immediately prior to, or following, competition.

In other aspects of the present invention there is provided a test kit for screening a sample for the presence of a ligand, which ligand is capable of forming a complex with a steroid hormone receptor and eliciting a genomic response when in a cell, the test kit comprising:
(i) a cell lysate comprising an androgen receptor that is capable of forming a ligand-receptor complex with a ligand from the test sample; or
(ii) a cell lysate comprising an estrogen receptor that is capable of forming a ligand-receptor complex with a ligand from the test sample, wherein the estrogen receptor is estrogen receptor alpha or estrogen receptor beta; or
(iii) a cell lysate comprising a progesterone receptor that is capable of forming a ligand-receptor complex with a ligand from the test sample, wherein the progesterone receptor is progesterone receptor A or progesterone receptor B; or
(iv) a cell lysate comprising a mineralocorticoid receptor that is capable of forming a ligand-receptor complex with a ligand from the test sample; or
(v) a cell lysate comprising a glucocorticoid receptor that is capable of forming a ligand-receptor complex with a ligand from the test sample; and
(vi) a nucleic acid molecule comprising:
   (a) a T7 polymerase promoter sequence comprising or consisting in SEQ ID NO: 85;
   (b) a response element that is capable of being bound by the receptor-ligand complex; and
   (c) a reporter construct
   where the response element (b) is located between the promoter sequence (a) and the reporter construct (c), and (a), (b) and (c) are operably linked; and optionally, a T7 polymerase.

In other aspects of the present invention there is provided a test kit for screening a sample for the presence of a ligand, which ligand is capable of forming a complex with a steroid hormone receptor and eliciting a genomic response when in a cell, the test kit comprising:
(i) a cell lysate comprising an androgen receptor that is capable of forming a ligand-receptor complex with a ligand from the test sample; or
(ii) a cell lysate comprising an estrogen receptor that is capable of forming a ligand-receptor complex with a ligand from the test sample, wherein the estrogen receptor is estrogen receptor alpha or estrogen receptor beta; or
(iii) a cell lysate comprising a progesterone receptor that is capable of forming a ligand-receptor complex with a ligand from the test sample, wherein the progesterone receptor is progesterone receptor A or progesterone receptor B; or
(iv) a cell lysate comprising a mineralocorticoid receptor that is capable of forming a ligand-receptor complex with a ligand from the test sample; or
(v) a cell lysate comprising a glucocorticoid receptor that is capable of forming a ligand-receptor complex with a ligand from the test sample; and
(vi) at least one steroid hormone receptor coactivator;
(vii) at least one steroid hormone receptor; and
(ix) a nucleic acid molecule comprising:
   (a) a T7 polymerase promoter sequence comprising or consisting in SEQ ID NO: 85;
   (b) a response element that is capable of being bound by the receptor-ligand complex; and
   (c) a reporter construct
   where the response element (b) is located between the promoter sequence (a) and the reporter construct (c), and (a), (b) and (c) are operably linked; and
(x) optionally, a T7 polymerase.

In yet further aspects of the present invention there is provided a test kit for screening a test sample for the presence of a ligand capable of eliciting a steroid hormone genomic response, the test kit comprising:
(i) a cell lysate comprising an androgen receptor that is capable of forming a ligand-receptor complex with a ligand from the test sample; or
(ii) a cell lysate comprising an estrogen receptor that is capable of forming a ligand-receptor complex with a ligand from the test sample, wherein the estrogen receptor is estrogen receptor alpha or estrogen receptor beta; or
(iii) a cell lysate comprising a progesterone receptor that is capable of forming a ligand-receptor complex with a ligand from the test sample, wherein the progesterone receptor is progesterone receptor A or progesterone receptor B; or
(iv) a cell lysate comprising a mineralocorticoid receptor that is capable of forming a ligand-receptor complex with a ligand from the test sample; or
(v) a cell lysate comprising a glucocorticoid receptor that is capable of forming a ligand-receptor complex with a ligand from the test sample; and
(vi) a nucleic acid molecule comprising:
   (a) a T7 polymerase promoter sequence comprising or consisting in SEQ ID NO: 85;
   (b) a response element that is capable of being bound by the receptor-ligand complex as defined in any of (i) to (v) above; and
   (c) a reporter construct,
   where the response element (b) is located between the promoter sequence
   (a) and the reporter construct (c), and (a), (b) and (c) are operably linked; and
(vii) optionally, a T7 polymerase; and
(viii) nucleoside triphosphates.

In yet further aspects of the present invention there is provided a test kit for screening a test sample for the presence of a ligand capable of eliciting a steroid hormone genomic response, the test kit comprising:
(i) a cell lysate comprising an androgen receptor that is capable of forming a ligand-receptor complex with a ligand from the test sample; or
(ii) a cell lysate comprising an estrogen receptor that is capable of forming a ligand-receptor complex with a ligand from the test sample, wherein the estrogen receptor is estrogen receptor alpha or estrogen receptor beta; or
(iii) a cell lysate comprising a progesterone receptor that is capable of forming a ligand-receptor complex with a ligand from the test sample, wherein the progesterone receptor is progesterone receptor A or progesterone receptor B; or
(iv) a cell lysate comprising a mineralocorticoid receptor that is capable of forming a ligand-receptor complex with a ligand from the test sample; or
(v) a cell lysate comprising a glucocorticoid receptor that is capable of forming a ligand-receptor complex with a ligand from the test sample; and
(vi) at least one steroid hormone receptor coactivator; and/or
(vii) at least one steroid hormone receptor corepressor; and
(viii) a nucleic acid molecule comprising:
   (a) a T7 polymerase promoter sequence comprising or consisting in SEQ ID NO: 85;
   (b) a response element that is capable of being bound by the receptor-ligand complex as defined in any of (i) to (v) above; and
   (c) a reporter construct,
   where the response element (b) is located between the promoter sequence (a) and the reporter construct (c), and (a), (b) and (c) are operably linked; and
(ix) optionally, a T7 polymerase; and
(x) optionally, nucleoside triphosphates.

In certain examples according to the assays, methods and test kits of the present invention, the nucleic acid molecules include one or more copies of various components of the nucleic acid, including the response element or reporter construct. For example, the reporter constructs or the nucleic acid molecules may include a single copy or multiple copies of the nucleic acid response elements including, but not limited to, duplicate copies, triplicate copies, quadruple copies etc.

In another example of the present invention, the steroid hormone receptor is purified from a cell, or is derived from a cell-based hormone receptor through recombinant cloning, expression and purification. In a further example, the steroid hormone receptor is synthetic, and its sequence modeled on, or evolved from, endogenous steroid hormone receptor sequences known in the art.

A person skilled in the art would also recognize that any steroid hormone receptor may be employed in the test kits, assays and methods of the present invention, provided that it retains the ability to bind to, and be activated by, a ligand of interest for detection. This includes, steroid hormone receptors, based on endogenous cellular forms, as well as recombinant or synthetic forms.

As such, the test kits, assays and methods according to the present invention may be configured to screen/detect any ligand that elicits a steroid hormone genomic response. However, a person skilled in the art will recognise that, according to the various assays concepts described herein, detection of different hormone classes (i.e. ligands) requires the format of the test kits, assays and methods to be properly configured and optimized. For example, detection of a ligand that binds to and activates an androgen receptor, such as testosterone as well as other testosterone-like hormones, requires test kits/assays comprising androgen receptor together with an androgen response element capable of binding to an activated androgen-receptor complex etc.

Designer steroids and non-steroidal anabolic drugs pose a significant and growing challenge for anti-doping laboratories. First identified in the early 2000s with the detection of tetrahydrogestrinone and madol, the threat posed by designer anabolic drugs has rapidly increased to include numerous potential agents. These synthetically-derived anabolic drugs are designed to evade detection or legal controls with respect to both manufacture and supply, and many are widely available on the internet where they are sold as so-called “supplements”.

Mass spectrometry remains the primary technology for the identification of known illicit steroid hormones and non-steroid anabolic drugs in biological samples and/or supplements. Despite its sensitivity and specificity, mass spectrometry remains limited by requiring prior knowledge of the steroid and non-steroid anabolic drug's chemical structures for detection. Moreover, mass spectrometry fails to provide information about the biological activity of the anabolic drugs detected, and is unable to differentiate between bioactive and inactive molecules. This is information that is required for legal prosecution of athletes, coaches, trainers, managers and manufacturers.

In recent years, yeast and mammalian cell-based in vitro androgen bioassays have been used to detect the presence of novel synthetic androgens, the androgenic potential of progestins as well as androgens, pro-androgens, designer androgens and designer non-steroid anabolic drugs in supplements. However, these assays suffer limitations associated with molecular complexity, as described elsewhere herein, and require technical skills that are both molecular and cellular in nature, are time consuming, labour intensive and expensive. As such, it is not feasible to consider the assays in their present form for inclusion in routine screening. In other words, yeast and mammalian cell-based assays suffer significant limitations because they are not high throughput or cost effective.

Advantageously, the present invention provides activity based test kits, assays and methods that work fundamentally on the principle of steroid hormone receptor activation. By detecting steroid hormone receptor activation by a ligand present within a sample to be tested, the present invention provides cell-free test kits, assays and methods that do not rely on structural knowledge of the ligand(s) being interrogated, can readily distinguish between the presence of biologically active and inactive ligands, and provide cost-effective, reliable and reproducible systems that do not require complex laboratory equipment or particular expertise to perform.

Accordingly, in an example according to the test kits, assays and methods described herein, the ligand is a performance enhancing designer drug and/or steroid.

In another example according to the test kits, assays and methods described herein, the ligand is of an unknown chemical structure.

In a further example according to the test kits, assays and methods described herein, the ligand is of a previously unknown chemical structure.

The present invention further contemplates use of the test kits, assays and methods as described herein for detecting antagonists of a target ligand by screening a sample of interest for a compound that will prevent binding of the ligand to its steroid hormone receptor such that it no longer activates the receptor and elicits a genomic response.

This is particularly useful when there is a need to screen for antagonists that block steroid hormone receptor activation (e.g.) as potential therapeutics for the treatment of endocrine and non-endocrine cancers. For example, the activity based test kits, methods and assays comprising one or more estrogen receptors according to the present invention can be used to screen compound libraries for the presence of antagonists or to monitor the loss of estrogen receptor activation in breast cancer tissue or blood in patients on cancer therapy.

In yet a further example according to all aspects of the test kits, assays and methods described herein, the biological sample is derived from an animal selected from the group consisting of equine, canine, camelid, bovine, porcine, ovine, caprine, avian, simian, murine, leporine, cervine, piscine, salmonid, primate, and human.

In yet a further example according to all aspects of the test kits, assays and methods described herein, the test sample is derived from biological material selected from the group consisting of urine, saliva, stool, hair, tissues including, but not limited to, blood (plasma and serum), muscle, tumors, semen, etc.

In yet a further example according to all aspects of the test kits, assays and methods described herein, the test sample is derived from a food selected from the group consisting of vegetable, meat, beverage including but not limited to sports drink and milk, supplements including, but not limited to, food supplements and sports supplements, nutritional supplements, herbal extracts, etc.

In yet a further example according to all aspects of the test kits, assays and methods described herein, the test sample is derived from a medication selected from the group consisting of drug, tonic, syrup, pill, lozenge, cream, spray and gel.

In yet a further example according to all aspects of the test kits, assays and methods described herein, the sample is derived from the environment selected from the group consisting of liquid, water, soil, textile including, but not limited to, plastics and mineral.

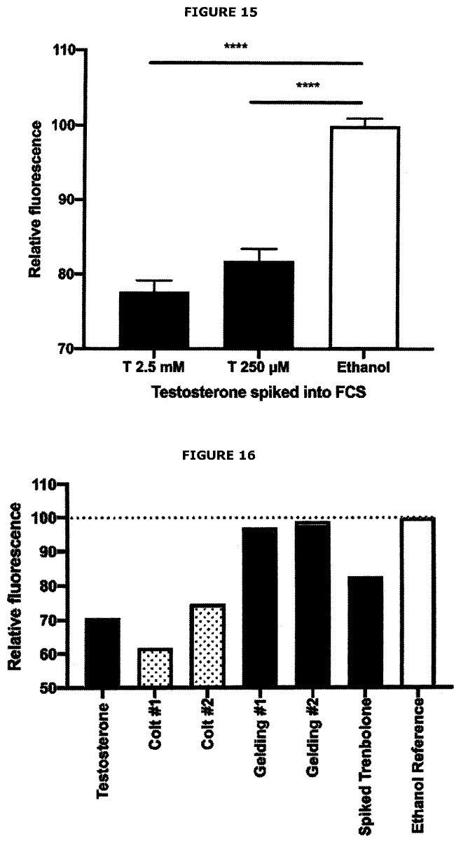

The information presented in Example 6 read in conjunction with FIGS. 14-16, demonstrates that the test kits and assay methods were used to detect testosterone (as an exemplary steroid hormone ligand) from biological matricies such as serum derived from calves and horses (FIG. 14), as well urine obtained from colts (FIGS. 15 and 16). These data reinforce the utility of the test kits and assay methods according to the present invention in the field, for example, in determining the doping status of human and equine athletes trackside, or for performing analysis on food supplements as part of export/import quality control.

Accordingly, in another example, the sample is a biological sample. In a related example, the biological sample is a body fluid sample, including but not limited to, blood, plasma, serum, saliva, interstitial fluid, semen and urine.

In another example, the sample derived from a plant, including but not limited to, leaf, flower, stem, bark, root, bud, pod, pollen and seed.

In another example, the sample is derived from an animal including, but not limited to, an equine animal, a canine animal, a dromedary animal, a bovine animal, a porcine animal, an ovine animal, a caprine animal, an avian animal, a simian animal, a murine animal, a leporine animal, a cervine animal, a piscine animal, a salmonid animal, a primate animal, and a human animal.

In another example, the test sample is a non-biological sample. In a related example, the non-biological sample includes, but is not limited to, a liquid sample including water, a soil sample, a textile sample including but not limited to plastics, a mineral sample, a food sample and a medication.

Examples of a food sample includes, but is not limited to, vegetables, meats, beverages, supplements and herbal extracts.

Examples of a medication includes, but is not limited to, drugs, tonics, syrups, pills, lozenges, creams, sprays and gels.

The invention is further described with reference to the following examples. It will be appreciated that the invention as claimed is not intended to be limited in any way by these examples.

### EXAMPLES

The information and data which follows demonstrates various prototype assays with respect to the detection of ligands that bind to and activate androgen receptors including (e.g.) Testosterone and Dihydrotestosterone, or ligands that bind to and activate estrogen receptors including (e.g.) Estradiol. These Examples are used to illustrate the activity assay platform described and claimed herein, where the assay concepts and principles exemplified by the detection of ligands that bind to androgen receptors or ligands that bind to estrogen receptors (i.e. ER-α and ER-β) would apply equally to the detection of other receptor ligands of interest including, without limitation, ligands that bind to progesterone receptor including but not limited to progesterone, ligands that bind to mineralocorticoid receptor including but not limited to aldosterone, and ligands that bind to the glucocorticoid receptor including but not limited to cortisol.

### Example 1

### Androgen Assay Prototype 1: Assay Architecture & Results

### 1.1 In Vitro Transcription Platform

Applicants initially developed an in vitro transcription platform include a DNA construct that encodes a T7 RNA consensus promoter sequence upstream of a tandem array of 3× androgen response elements (ARE) upstream of an RNA aptamer sequence for Mango II, combined with recombinant androgen receptor (AR), recombinant heat shock protein 90 (HSP90), T7 RNA polymerase, nucleoside triphosphates and a transcription buffer. The T7 promoter will drive a high level of RNA aptamer expression that will be detected by binding to fluorophore, Thiazole Orange 1—biotin (TO1). Inhibition of T7-driven aptamer expression will occur when ARE is bound by ligand-activated AR.

### Androgen Response Element (ARE)

The androgen response element tested in these experiments was a tandem array of 3×ARE

### Androgen Receptor (AR)

Commercial recombinant AR has been tested from Sigma-Aldrich

### T7 RNA Polymerase

The MegaScript kit from ThermoFisher was used in these studies, as a source of T7 RNA polymerase, and necessary buffers and nucleotide triphosphates.

### 1.2 Description of ARE-Mediated Transcription/Translation

Natural androgen signaling starts with the diffusion of an androgenic molecule into the cell where it binds to the AR being held in an inactive state in the cytoplasm, bound to heat shock protein 90 (HSP90). Upon binding the androgenic molecule (or ligand), AR undergoes a conformational change that releases HSP90, and exposes nuclear localization and dimerization sites. The ligand-AR complex is targeted for translocation to the nucleus where AR binds to ARE sites in the DNA and RNA polymerase II holoenzyme assembles and initiates transcription of AR-regulated genes. Transcription of a gene by RNA polymerase II produces an mRNA transcript that, in turn, acts as the template for the ribosome machinery to make a protein.

Exploiting this natural biology, the experiments described now show testosterone (a natural androgen)—activated AR decreases T7-mediated transcription, as ARE has been placed downstream of the T7 promoter (and transcription initiation site) so when testosterone-liganded AR binds to the ARE, there is a reduction/inhibition in T7-mediated transcription.

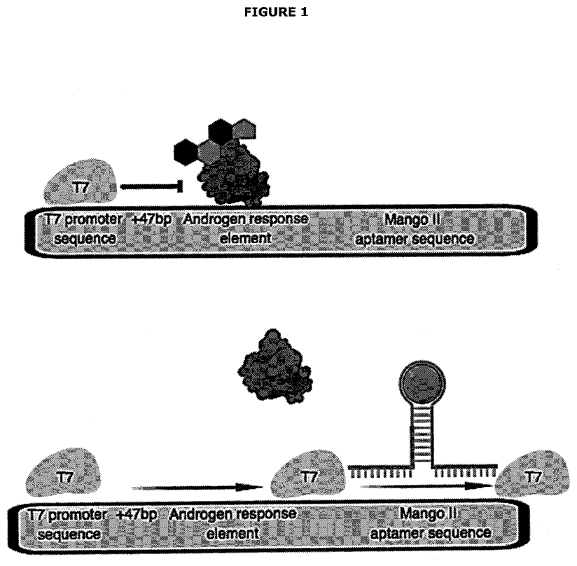

FIG. 1 illustrates a schematic of ARE-mediated blocking of T7 RNA polymerase-mediated transcription.

In the presence of ligand, such as the natural androgen, testosterone, AR binds to ARE and inhibits transcription by T7 polymerase. In this state, less RNA Mango II aptamer is formed.

In the absence of ligand, AR is not activated to bind to ARE, and therefore the DNA is free from an obstructive protein. T7 proceeds along the DNA construct to generate RNA Mango II aptamer, which is subsequently detected by TO1-B binding and fluorescence.

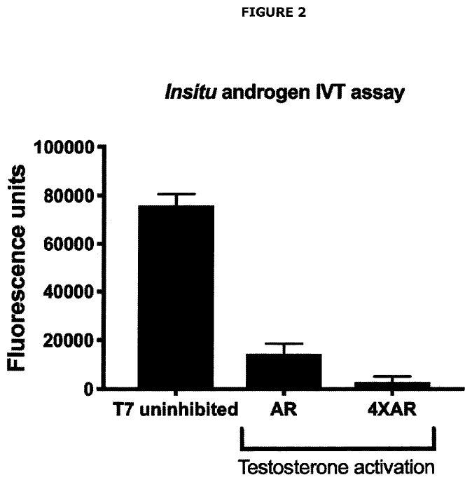

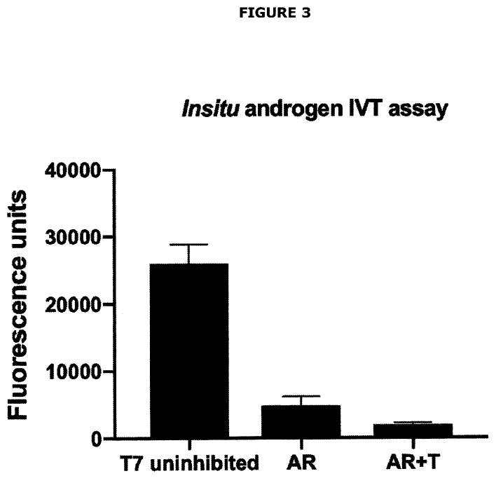

In reference to the result presented in FIGS. 2 and 3, testosterone-activation of AR blocking of transcription was investigated. No AR shows full T7-mediated transcription and RNA Mango II aptamer expression. This is reduced in the presence of AR, and further reduced when AR is activated by 11.5 ng/ml testosterone.

In reference to FIG. 4, it was next investigated if HSP90 could inhibit the baseline activation of AR. AR is activated by conformational change through activation of region activation factor-1 (AF-1) via testosterone, or via auto-activation of region activator factor-2 (AF-2). For AR, AF-2 is very active and can represent up to 50% of AR activity. To suppress AF-2 auto-activation, HSP90 was added to keep AR in an inactivated state that would not bind to ARE in the absence of ligand. In the presence of ligand, however, HSP90 should competitively dissociate from the AR, and the liganded-AR should bind to the ARE.

### Example 2

### Androgen Assay Prototype 2: Assay Architecture & Results

The concept of the Androgen Assay Prototype 2 is that a single protein RNA polymerase, such as T7 RNA polymerase, binds to its promoter sequence on a DNA template. The DNA template encodes the RNA aptamer Mango II sequence downstream of the promoter. A hormone response element (HRE) is located between the T7 promoter and the Mango II sequence. When a steroid hormone receptor (SHR) is added to the T7 in vitro transcription (IVT) reaction mix and activated by a receptor-specific ligand, the SHR binds to the HRE on the DNA template and in this bound position physically inhibits T7 RNA polymerase from transcribing the DNA into the RNA, and therefore no Mango II aptamer is formed. The formation of Mango II is detected by adding a specific fluorophore, such as TO1-biotin, to the reaction mix which binds to the Mango II aptamer. Upon binding to the Mango II aptamer, TO1 emits an excitation wavelength at 535 nm wavelength when excited by 510 nm wavelength. Fluorescence is measured using a standard fluorimeter.

### 2.1 DNA Sequences Used in Development of Androgen Assay Prototype 2

The following experiments use AR or ERc as the example SHR and the ARE or ERE as the example HRE.

The key step that underpins T7-mediated in vitro transcription is for the recognition of its promoter sequence in the DNA template. To then measure that T7 transcription has occurred the DNA sequence can encode a reporter enzyme (protein) or a reporter RNA (e.g. aptamer). In the following examples, the DNA sequence encoded a reporter RNA (Mango II).

Commercial DNA fragment synthesis was used to generate a series of DNA templates that encoded the (1) T7 initiator sequence, (2) ARE, (3) Mango II RNA aptamer with F30 scaffold. These DNA fragments were cloned into a plasmid and amplified using transformed E. coli. Subsequent plasmid extraction, purification and linearization provided the final DNA template for Prototype Assay. Other examples where transcription factors have blocked T7 activity show that ˜10-32 bp between the T7 initiator sequence and the transcription factor site is optimal for blocking T7 progress. Therefore, a 12 bp filler sequence was included in the DNA fragment.

TABLE 2
Sequences Used in Prototype 2 for Detection of Androgenic Ligands

| Component | Sequence |
|---|---|
| T7 initiator sequence | TAATACGACTCACTATAG (SEQ ID NO: 1) |
| 12 bp filler | ACTCTGGAGGAA (SEQ ID NO: 74) |
| 3XtandemARE | AAGCTTAGAACAGTTTGTAACGAGCTCGTTACAAACTGTTCTAGC |
| | TCGTTACAAACTGTTCTAAGCT (SEQ ID NO: 75) |
| Primary ARE | TGGAGAACAGCCTGTTCTCCA (SEQ ID NO: 76) |
| MangoIIF30scaffold* | TTGCCATGTGTATGTGGGTACGAAGGAGAGGAGAGGAAGAGGAGAGTAC |
| | CCACATACTCTGATGATCCTTCGGGATCATTCATGGCAATCTAGGA |
| | (SEQ ID NO: 77) |
| MangoII | TACGAAGGAGAGGAGAGGAAGAGGAGAGTA (SEQ ID NO: 78) |

*single underline region, Mango II

### 2.2 Testosterone-Activated AR is Able to Suppress T7-Mediated Expression of RNA Aptamer, Mango II

The SHR-HRE-RNA aptamer reaction to detect a SHR ligand hinges on the blockade of T7 transcription by an SHR bound to a hormone response element. In the following examples, AR and ARE were used to represent SHR and HRE.

TABLE 3
Reaction components required for the AR/HSP90-ARE
reaction with T7-generated Mango II as readout

| Component | Concentration/Activity per reaction* | |
|---|---|---|
| T7 RNA polymerase | 0.5 | μl |
| T7 ARE MangoII DNA template | 100 | ng |
| T7 reaction buffer** | 2.5 | μl |
| AR | 50 | ng |
| HSP90 | 100 | ng |
| Steroid hormone | 250 | μM |
| Mango II-specific fluorophore | 100 | nM |

*concentrations for the initial experiments were based on previous work (e.g. FRET assays for AR/HSP90 or other cell-free assays established in the inventor's laboratory) or manufacturer's protocols (DNA template concentration, enzyme, and fluorophore)
**T7 reaction buffer (eg. NTP buffer from HiScribe kit [ThermoFisher Scientific] or 10× buffer supplied with T7 enzyme or home-made buffer)

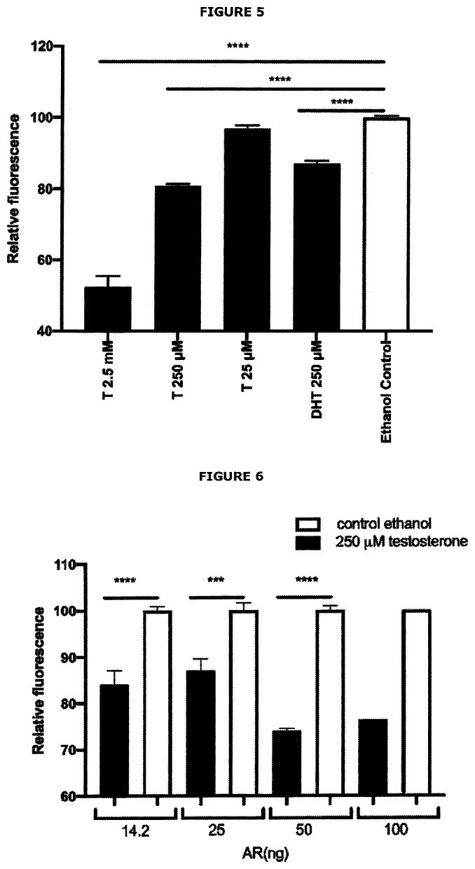

The reactions were assembled and initiated with the addition of T7 RNA polymerase and incubated for 150 mins at 37° C. During this time, RNA Mango II aptamer was generated. The detection of Mango II was by the addition of fluorophore, thiazole orange (TO1) and measuring output with fluorimeter, excitation 510 nm and emission 535 nm. FIG. 5 shows that testosterone-activated AR is able to reduce the amount of T7-generated RNA aptamer Mango II in a dose-dependent manner. Testosterone represents the most abundant endogenous circulating androgen in the body. Testosterone can be converted in peripheral tissues (e.g. gonads) to dihydrotestosterone (DHT). DHT was also found to activate AR-regulated reduction in T7-generation of Mango II.

### 2.3 Titration of the Steroid Hormone Receptor

As above, AR was used as the example steroid hormone receptor in the following experiments.

In the above experiment, and as shown in Table 3, the initial AR concentration was based on previous cell-free assays established in the inventors' laboratory. The reduction in T7-mediated Mango II generation is dependent on there being sufficient AR to block all the T7 enzyme molecules currently engaged with, and active on, the DNA templates. This means that both the ratios of AR to T7 and AR to DNA template are important. The level of AR, however, needs to be considered in the context of an optimal level of T7 and/or DNA template that supports sufficient Mango II generation required for detecting changes in fluorescence.

In the next series of experiments, the concentration of AR per reaction was altered to show the effects on Δ[Mango II].

TABLE 4
Titration of AR

| Component | Concentration per reaction | | | | | | | |
|---|---|---|---|---|---|---|---|---|
| T7 RNA polymerase | 0.5 | μl | 0.5 | μl | 0.5 | μl | 0.5 | μl |
| T7 ARE MangoII DNA | 100 | ng | 100 | ng | 100 | ng | 100 | ng |
| template | | | | | | | | |
| T7 reaction buffer | 2.5 | μl | 2.5 | μl | 2.5 | μl | 2.5 | μl |
| AR | 14.2 | ng | 25 | ng | 50 | ng | 100 | ng |
| HSP90 | 100 | ng | 100 | ng | 100 | ng | 100 | ng |
| Steroid hormone | 250 | μM | 250 | μM | 250 | μM | 250 | μM |
| Mango II-specific | 100 | nM | 100 | nM | 100 | nM | 100 | nM |
| fluorophore | | | | | | | | |

Using the 50 ng AR reaction as the “standard” reaction, the number of molecules of AR is 2.737e¹¹ (454.55 fmol or 22.72 nM) to the number of DNA molecules at 3.798e¹⁰ (63.06 fmol, or 3.15 nM) resulting in an excess ratio of AR:DNA of 7.2:1. Doubling the AR concentration to 100 ng, doubles the ratio to 14.4:1. Halving the AR concentration to 25 ng, halves the ratio to 3.6:1, and further again for the 14.2 ng AR to 1.8:1.

The data in FIG. 6 shows that the AR:DNA ratio of 7.2:1 or greater produces the greater ΔMango II. The lower ratios still show ΔMango II but the effect size is reduced. Notably the jump in ratio from 7.2 to 14.4 showed no improvement in effect size on ΔMango II suggesting that at 7.2:1 the excess of AR to DNA is sufficient for blocking T7 activity, and having a further excess creates redundancy.

When considering SHR activation by steroid hormones, it is also necessary to examine the ratio of HSP90 to SHR, as an excess of HSP90 will block activation of the SHR, especially at low concentrations of ligand, while insufficient HSP90 will allow autoactivation of the SHR. Again, using AR as the representative SHR, in the next set of experiments, the ratio of HSP90 to AR was altered to scrutinize effects on T7-generation of Mango II by testosterone.

TABLE 5
Titration of the HSP90:AR ratio

| Component | Concentration per reaction | | | | | | |
|---|---|---|---|---|---|---|---|
| T7 RNA | 0.5 μl | 0.5 μl | 0.5 μl | 0.5 μl | 0.5 μl | 0.5 μl | 0.5 μl |
| polymerase | | | | | | | |
| T7 ARE MangoII | 100 ng | 100 ng | 100 ng | 100 ng | 100 ng | 100 ng | 100 ng |
| DNA template | | | | | | | |
| T7 reaction buffer | 2.5 μl | 2.5 μl | 2.5 μl | 2.5 μl | 2.5 μl | 2.5 μl | 2.5 μl |
| AR | 14.5 ng | 25 ng | 50 ng | 100 ng | 100 ng | 50 ng | 25 ng |
| HSP90 | 100 ng | 100 ng | 100 ng | 200 ng | 100 ng | 50 ng | 25 ng |
| Steroid hormone | 250 μM | 250 μM | 250 μM | 250 μM | 250 μM | 250 μM | 250 μM |
| Mango II-specific | 100 nM | 100 nM | 100 nM | 100 nM | 100 nM | 100 nM | 100 nM |
| fluorophore | | | | | | | |

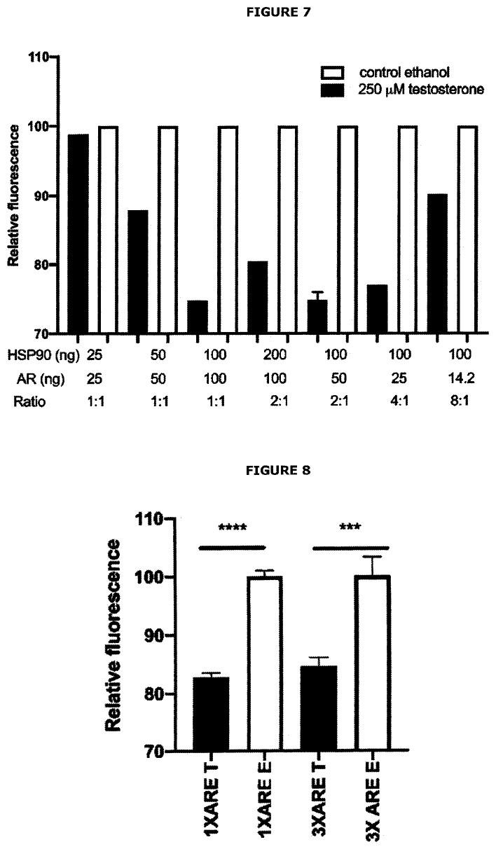

FIG. 7 shows that a standard reaction of 50 ng AR and 100 ng HSP90 with a ratio of HSP90:AR of 2.44:1 was optimal, however other ratios could achieve the same level of ΔMango II. For example, a 100 ng HSP90 corresponding to 6.69e¹¹ molecules (1.11 pmol or 55.5 nM) and 100 ng AR corresponding to 5.47e¹¹ molecules (909.09 fmol or 45.5 nM) or ratio of 1.22:1 also showed good suppression of T7-mediated generation of Mango II. Equally, AR (25 ng) and HSP90(100 ng) representing 1.37e¹¹ molecules (227.27 fmol or 11.4 nM) and 6.69e¹¹ molecules (1.11 pmol or 55.5 nM) respectively, or ratio of 4.88:1 showed a ΔMangoII that was not different to a ratio of 2.44:1. However, a 9.76:1 ratio showed reduced ΔMangoII, as did 1.22:1 ratios where AR concentration was <100 ng.

Importantly, the data from FIGS. 6 and 7 highlights a unique ability to stoichiometrically define a biological reaction.

AR is most effective at binding to an ARE and blocking T7 progress when the AR:DNA ratio is ≥7.2:1. AR is most effective at being activated by ligand and binding to an ARE when the HSP90:AR ratio is between 1.22:1 and 4.88:1.

### 2.4 Manipulating the HRE

The data so far has used a 3×ARE sequence commonly used in cell-based AR bioassays (see Table 2). However, the primary ARE that has been identified is of the sequence AGAACAgccTGTTCT. Considering the functioning of this assay whereby T7 enzyme is physically blocked by AR, it may not be necessary for the presence of 3×ARE sequences. In the following test, a single, but primary in sequence, ARE was cloned into the T7 DNA template. FIG. 8 clearly shows that the single ARE was as effective as the 3×ARE sequence with activated AR blocking T7 RNA polymerase.

### 2.5 Titrating the DNA Template

The DNA template is a critical component of the assay and needs to be at a concentration that supports T7 transcription while also not being in excess so that the number of AR molecules present can sterically hinder transcription of the reporter construct by T7 enzyme.

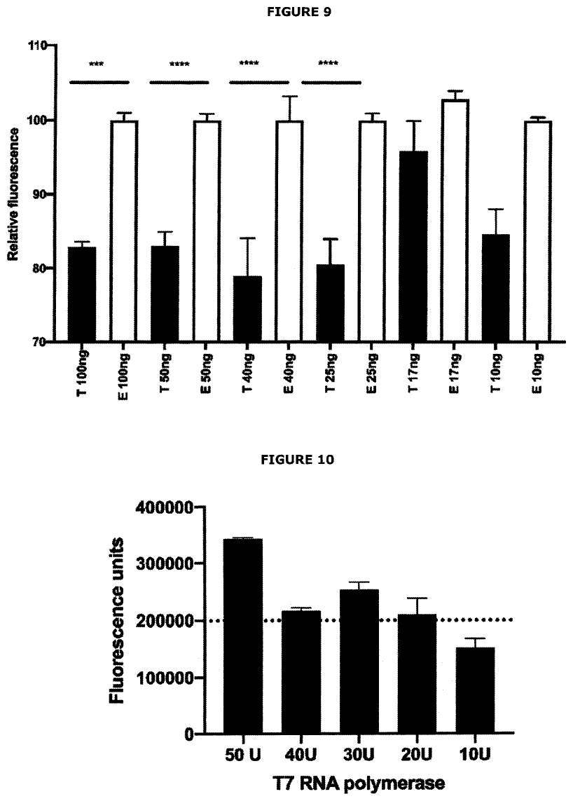

In the next series of experiments, the DNA template was titrated holding AR and T7 constant. This provided insight into the number of DNA molecules required to support transcription and to maintain AR blockade. FIG. 9 data shows that when the concentration of AR was 50 ng (2.737e¹¹ molecules, 454.55 fmol or 22.72 nM) and the DNA concentration was 100 ng (3.798e¹⁰, 63.06 fmol or 3.15 nM) there was good detection of ΔMango II, representing an AR:DNA ratio of 7.2:1. Increasing the amount of excess AR to DNA by lowering DNA concentration to test ratios of 14.4:1, 17.28:1 and 28.8:1 had no effect on improving ΔMango II. Thus, an excess ratio of 7.2:1 achieves maximal effect. Note, that reducing DNA template to 6.46e⁹ or 3.398e⁹ did affect ΔMango II, because there was a dramatic decrease in T7-mediated fluorescence output (data not shown). Thus, 9.495e⁹ molecules of double-stranded DNA is the minimum level required for a successful reaction, while an excess of AR:DNA must be maintained above 7.2:1. Going beyond 7.2 with addition of more AR creates redundancy, while decreasing DNA to increase the ratio risks T7-mediated transcription per se.

Data from FIG. 9 has continued to define a biologically effective, but synthetically achieved SHR reaction. Through the stoichiometric analysis of individual components, the experiments have confirmed that AR is most effective at binding to an ARE and blocking T7 progress when the AR:DNA ratio is >7.2:1. However, when making up this ratio it is necessary to lock the DNA template concentration at a minimum of 9.495e⁹ molecules of double-stranded DNA otherwise the base level of transcription is compromised.

### 2.5 Titrating T7 Polymerase

The final component of the Androgen Assay Prototype 2 that needs to be considered to allow a fully defined stoichiometric reaction that can mimic steroid hormone receptor biology is the T7 enzyme itself. T7 RNA polymerase is used in this reaction to generate the reporter, which in these examples is the RNA aptamer, Mango II.

The amount of T7 enzyme is critical to maintain the fluorescent readout within a spectrum that will allow an optimal dynamic range. T7 is an enzyme so it is not just concentration that is an important factor, but also activity. The next series of experiments showed the effect of altering T7 activity on ΔMango II.

In FIG. 10, data shows T7-generated MangoII:TO1B fluorescence levels as the T7 units are titrated from 50U to 10U. It is noted that in the enzyme range of 40-10U there is no clear difference in fluorescence measured with all measurements close to 200000 indicating a clear detection baseline, at which point sensitivity is not high enough to measure change in output. This suggests that in order to measure ΔMangoII, the fluorescence measurement must be greater than 200000.

To further demonstrate the effect of minimal fluorescence level, T7 (50U) or T7 (100U) was used to generate Mango II RNA aptamer, in a reaction blocked with testosterone-induced AR, or as control ethanol. The data in FIG. 11 shows that if 50U is used the fluorescence is 250000-300000, testosterone-activation shows ˜9% in ΔMango II however if the fluorescence is in the range >600000 the same reaction shows a ˜16% ΔMango II. Thus, the data highlights that T7 activity must be in the range that produces MangoII-TO1B fluorescence >300000. The exact activity or units will be dependent on batch-to-batch and/or supplier-to-supplier differences in the specific activity of recombinant T7 RNA polymerase.

### Example 3

### Estrogen Assay Prototype 3: Assay Architecture & Results

In the above examples, AR/ARE was used as the example SHR/HRE. The following series of experiments used the defined reaction stoichiometry established for AR/ARE to show the applicability of the test to other SHRs, in this case ERα. The results presented below demonstrate that estradiol-activated ERα is able to suppress T7-mediated expression of RNA aptamer, Mango II.

TABLE 6
Sequences Used in Prototype 3 for Detection of Estrogenic Ligands

| Component | Sequence |
|---|---|
| T7 initiator sequence | TAATACGACTCACTATAG (SEQ ID NO: 1) |
| 12 bp filler | ACTCTGGAGGAA (SEQ ID NO: 74) |
| Primary ERE | CAGGTCAGCATGACCTG (SEQ ID NO: 79) |
| MangoIIF30scaffold* | TTGCCATGTGTATGTGGGTACGAAGGAGAGGAGAGGAAGAGGAGAGTAC |
| | CCACATACTCTGATGATCCTTCGGGATCATTCATGGCAATCTAGGA |
| | (SEQ ID NO: 77) |
| Mango II | TACGAAGGAGAGGAGAGGAAGAGGAGAGTA (SEQ ID NO: 78) |

*single underline region, Mango II

The standard AR/ARE conditions that proved to be a successful detection test for testosterone were used, however AR was replaced with ERα and a DNA template encoding a single ERE replaced the ARE DNA template (Table 6). FIG. 12 shows that replacing AR with ERα (50 ng) in combination with HSP90 (100 ng) and activating with 5 μM estradiol (E2) led to a reduced MangoII:TO1 output. The ΔMango II was ˜60%. When considering ratios, ERα at 68 kDa is smaller than AR (110 kDa) and therefore the number of molecules added for weight was 4.428e11 (735.20 fmol or 36.8 nM). The ratio of HSP90:ERα therefore was 6.69e¹¹:4.428e¹¹ or 1.51:1 and ERα:DNA 4.428e¹¹:3.798e¹⁰ or 11.66:1. Both of these were in the range found to be successful ratios for detecting androgens with the AR version of the SHR/HRE test.

TABLE 7
ERα/ERE reaction

| Component | Concentration per reaction | |
|---|---|---|
| T7 RNA polymerase | 0.5 μl or 100U | |
| T7 ERE MangoII DNA | 100 | ng |
| template | | |
| T7 reaction buffer | 2.5 | μl |
| ERα | 50 | ng |
| HSP90 | 100 | ng |
| Mango II-specific | 100 | nM |
| fluorophore | | |

The importance of the ERα:ERE reactions for detection of an estrogen is two-fold. Firstly, it shows the simplicity in switching out the essential test components from AR/ARE DNA template to an ER/ERE DNA template. Secondly, it shows the defined stoichiometric reaction established for AR/ARE that mimics androgen biology by ligand binding to an androgen receptor, becoming displaced from HSP90, and binding to an ARE is translatable to a second steroid hormone receptor/steroid response element combination.

The data shows that the test is able to recapitulate steroid hormone biology in a cell-free manner and in such a way that every component can be defined—a truly in situ test. This is unlike the situation in vitro when using cell-based bioassays or cell-free bioassays based on nuclear extracts providing the holoenzyme RNA polymerase II. In the case of cell-based bioassays, the levels of SHR, HSP90, DNA template, and RNA polymerase II cannot be defined at all because they are influenced by the expression pattern of the cell. Cell-free bioassays based on nuclear extracts can, in part, define the stoichiometry of a reaction by describing SHR, HSP90 and HRE levels, however are unable to define the RNA polymerase level. RNA polymerase II is a holoenzyme, made up of several subunits or proteins, and therefore can only be supplied in the form of a nuclear extract. The nuclear extract is undefined in what other proteins are present. In this single polypeptide RNA polymerase form of the assay, the stoichiometry of the reaction can be fully defined and the data shows that such reactions can by synthetically manipulated to mimic the natural biology of steroid hormone receptors.

### Example 4

### Ligand-Shr/Hre Stoichiometric Reactions for Prototype Assays

The data presented in Examples 2 and 3 has revealed defined stoichiometry that mimics steroid hormone receptor biology with ligand binding to a specific receptor thereby the receptor displaces from HSP90 and binds to a steroid response element—the classical steroid hormone genomic response. In nature, the SHR will activate or repress the expression of the target gene. In the Prototype Assays described herein, binding of the liganded-SHR represses expression of the reporter molecule.

TABLE 8
Ratio of molecules providing optimized
output for Prototype Assays

| | Ratio Androgen | |
|---|---|---|
| Components | Assay Prototype 2 | Figure (data) |
| SHR:DNA template | 6:1-14.4:1 | FIG. 6, 9 & 12 |
| | (>8 superfluous) | |
| SHR: HSP90 | 1.22:1-4.88:1 | FIG. 7 & 12 |
| T7 activity | >600000 optimal (50-100U T7), | FIG. 10, 11 |
| (TO1B fluorescence | >200000 threshold (25U T7) | |
| readout) | | |
| HRE (copy number) | 1X | FIG. 8 |

TABLE 9
Template showing example stoichiometry
of an SHR/HSP90/HRE reaction

| | Amount | |
|---|---|---|
| | Androgen Assay | Concentration |
| Components | Prototype 2 | (nM) |
| SHR (# molecules) | 2.737e¹¹-5.474e¹¹ | 22.7-45.5 |
| HSP90 (#molecules) | 6.69e¹¹ | 55.5 |
| DNA template | 9.459e⁹-3.798e¹⁰ | 0.79-3.15 |
| T7 RNA polymerase* | X units or μl | N/A |

*X defined by the volume/units (supplier dependent) of T7 RNA polymerase required to generate Mango II:TO1B fluorescence of >600000 units. Absolute baseline threshold of activity >200000 fluorescence.

### Example 5

### Androgen Assay Prototype 2 Detects Androgenic Molecules

Androgenic molecules are primarily steroid hormones. Testosterone and dihydrotestosterone are the most abundant endogenous androgens. Based on their structures, synthetic androgenic anabolic steroids (AAS) have been designed and marketed. AAS are the most commonly abused performance enhancing drug in athletes, human and animal alike. Another class of androgenic molecules that have been synthetically derived are the selective androgen receptor modulators or SARMs. Like AAS, SARMs are abused by athletes. Both AAS and SARMs differ in their structures, with a great variety of different side groups and backbones. This next series of experiments tested whether the AR/HSP90-ARE Prototype Assay was able to detect different AAS and SARMs.

TABLE 10
AAS and SARMs tested with Androgen Assay Prototype 2

| Androgenic compound | Nature of compound |
|---|---|
| Testosterone | Endogenous |
| Dihydrotestosterone | Endogenous |
| 17α-trenbolone | AAS |
| 17β-trenbolone | AAS |
| TRENA | AAS-designer steroid-internet sourced |
| Altrenogest | Progestin, newly described androgen |
| Trendione | AAS |
| Nandrolone | AAS |
| Boldenone | AAS |
| 93746 | SARM |
| BMS | SARM |
| LDG | SARM |
| ACP105 | SARM |
| YK-11 | SARM |
| Andarine | SARM |
| Ligandrol | SARM |
| Ostarine | SARM |

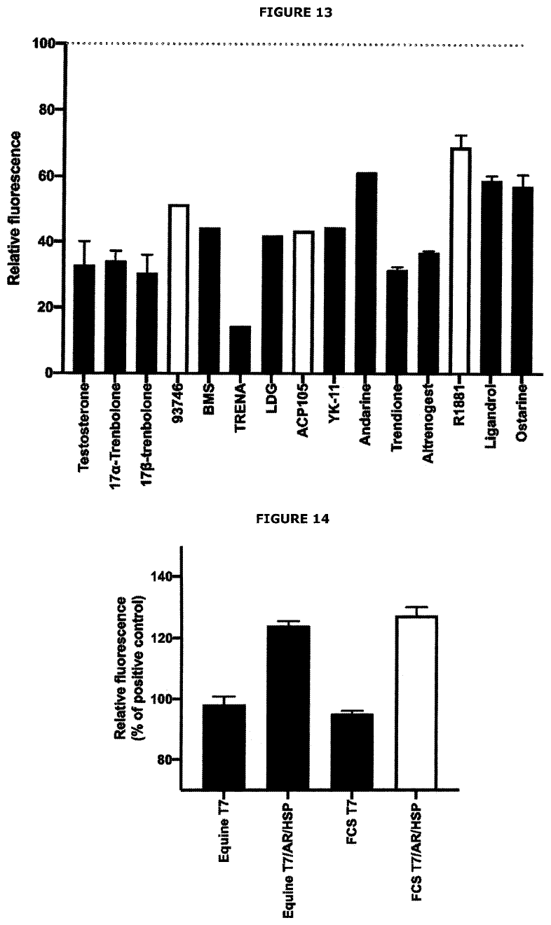

FIG. 13 shows that the assay was able to detect a variety of AAS and SARMs.

### Example 6

### Androgen Assay Prototype 2 Detects Androgenic Molecules in a Biological Matrix

Androgen Assay Prototype 2 was next tested for its ability to detect testosterone when present in a biological matrix, such as serum or plasma. First, it was necessary to demonstrate that T7 RNA polymerase continued to operate in the presence of serum, that is serum per se did not suppress T7 efficacy in generating Mango II aptamer. FIG. 14 shows that in the presence of equine serum or fetal calf serum (FCS) T7 RNA polymerase continued to generate Mango II.

There was no evidence that serum inadvertently suppressed T7 activity. Next, it was tested whether in the presence of serum, AR remained responsive to testosterone as the example ligand. Reactions were established as described above, except this time water component was replaced with serum into which testosterone (or ethanol as vehicle) had been spiked. FIG. 15 shows that the reaction is not compromised if serum is present as a reaction component. This result is very important as shows the assay is capable of detecting androgen levels in a biologically relevant sample for clinical or sports doping application, for example.

In the next phase of testing, the Androgen Assay Prototype 2 was tested for its ability to detect endogenous androgens deconjugated and extracted from equine urine samples. Racehorse urine samples were collected on race day and steroids deconjugated and extracted using routine processes. The extracted steroids were resuspended in ethanol and subjected to the assay.

FIG. 16 shows that the Androgen Assay Prototype 2 was able to detect high levels of androgens in the urine samples from colts (male horses) while not being able to detect high levels from the geldings (castrated male horses). Testosterone and spiked trenbolone (AAS) in gelding urine were used as controls.

Data from FIG. 15 and FIG. 16 show that the assay is able to detect androgenic molecules in biological matrices including serum and urine.

### Example 7

### Utility of a Cell Lysate as a Source of Steroid Hormone Receptor

The main components of the androgen screening assay are the steroid hormone response element (HRE) and the steroid hormone receptor (SHR), that is held in an inactive state by a coregulatory protein in the absence of ligand. In the presence of ligand, the regulator protein is released from the SHR, and the liganded-SHR binds to the HRE that is encoded on a double-stranded DNA reporter construct.

In this Example, the DNA reporter construct encodes an RNA polymerase promoter site, an HRE, and an RNA aptamer, although the skilled person would recognize that fluorophore bound RNA aptamer could be replaced by any other reporter construct, including the other examples described herein.

In this Example, the RNA polymerase is the T7 enzyme, a recombinant single protein RNA polymerase. The DNA reporter construct then encodes downstream from the T7 site, a series, or a single, HRE, with an RNA aptamer reporter located further downstream. When T7 is active, it will bind to its promoter and transcribe this DNA fragment producing an RNA aptamer. This RNA aptamer acts as the reporter. When this T7 reaction is performed in the presence of a steroid hormone, the steroid hormone will bind to its SHR, dislodge the coregulatory protein, and the steroid hormone-SHR will bind to the HRE. In this position, the SHR will physically inhibit T7 RNA polymerase from transcribing the RNA aptamer. Therefore, the fluorescence readout is reduced in the presence of a steroid hormone ligand.

The key components of the assay include the T7 RNA polymerase and the recombinant SHR protein. For purposes of producing an assay that is cost-effective, rapid and reproducible, the activity of both the RNA polymerase and the SHR protein were interrogated, with the example of SHR being the androgen receptor (AR). However, a person skilled in the art would appreciate that androgen receptor could be switched for any steroid hormone receptor, including estrogen hormone receptor to achieve the same or similar outputs to those outlined below

The reaction mix used in the experiments which follow is reflected in Table 11.

TABLE 11
Assay Components

| Component | Concentration per reaction | |
|---|---|---|
| T7 RNA polymerase (8U/μl) | 0.5 | μl |
| T7 ARE MangoII DNA template | 100 | ng |
| T7 reaction buffer | 2.5 | μl |
| AR | 25 | ng |
| HSP90 | 50 | ng |
| Mango II-specific fluorophore | 100 | nM |

Recombinant AR protein is costly to produce in the quantities needed for scale-up production. Recombinant AR is also difficult to handle as it requires cold temperature storage and its large size renders it susceptible to degradation. Recombinant AR protein is expressed in genetically modified cells where the host cells maybe yeast, insect, bacteria, or mammalian. Once expressed within the cell, a lengthy process is performed to purify recombinant AR from all other cellular proteins. As the AR protein is a large protein of 110 kDa, the purification process is made even more difficult to ensure full-length and active protein. Understandably, there is substantial cost to the purchase, or production, of this protein.

In the cell, inactive AR is located in the cytoplasm or in its ligand-activated state, AR is found in the nucleus. In over-expression cell systems whereby, the plasmid encodes a highly active constitutive promoter, AR is expressed at much higher levels than normal, even in the absence of androgens leading to large amounts of inactive AR.

One of the first steps when purifying recombinant AR protein is to produce a cell lysate. This cell lysate contains all cellular material including the proteins. The over-expressed AR will be in this pool of proteins.

To override the need to use purified recombinant AR protein in the androgen screening assay, Applicants tested whether a whole cell lysate from HEK293 cells could act as the source of AR. A commercially available transient overexpression lysate of AR (transcript variant 1, NM_000044, OriGene, LY400012) where the expression host was HEK293T cells (human embryonic kidney cells) was used. As the AR concentration is not known, the experiments initially used the lysate at a protein concentration of 25 ng/μl. FIG. 18 shows that the AR cell lysate suppressed T7 RNA polymerase activity when activated with testosterone.

The absolute concentration of AR is not known in the AR cell lysate. The AR lysate concentration that was most effective in the reaction was next empirically determined. The AR lysate was titrated from 150 ng/μl to 6.25 ng/μl. The results show that the slope of the line was highest for 40 ng/μl of lysate, with a range of 30-50 ng/μl being the strongest (FIG. 19, Table 12). This result suggests that for this AR lysate batch, a concentration of 30-50 ng/μl provides AR at the required concentration to adequately suppress T7 RNA polymerase activity.

TABLE 12
Slopes of linear regression plots from FIG. 18

| AR lysate conc. | | Fold difference |
|---|---|---|
| (ng/μl) | Slope of line | (25 ng/μl) |
| 6.25 | −95666 | 0.625 |
| 12.5 | −164873 | 1.077 |
| 25 | −153132 | 1 |
| 30 | −301788 | 1.971 |
| 40 | −322852 | 2.108 |
| 50 | −282390 | 1.844 |
| 100 | −182931 | 1.195 |

Previously, it has been determined that a HSP90:AR ratio of 2:1 is optimal, however a range 8:1 is tolerated with probable redundancy. Using an AR lysate it is not possible to determine absolute AR concentration and subsequently, determine a 2:1 HSP90:AR ratio. Instead, the ratio was empirically derived with the ratio based on the total protein concentration of the cell lysate and a HSP90:AR lysate of 2:1 based on ng/μl was maintained (FIG. 19). This will maintain a >2:1 ratio in all reactions.

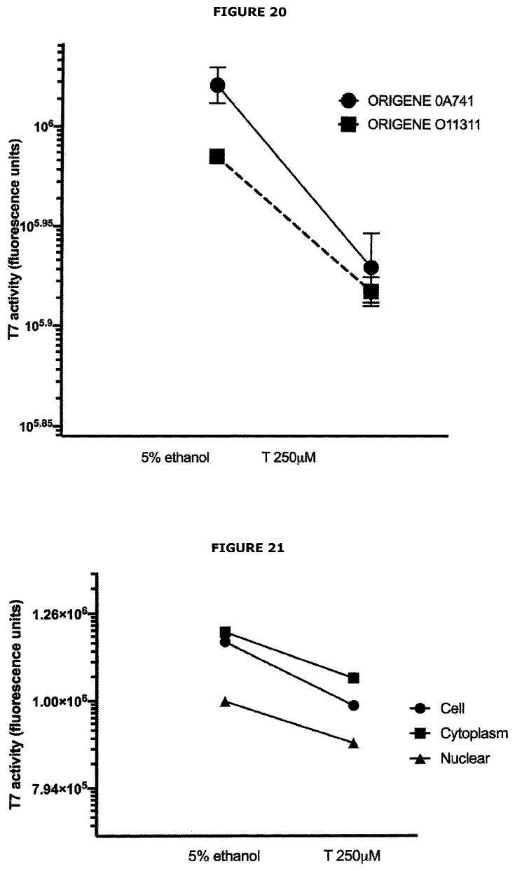

Together, these data show that the AR lysate concentration used in the assay can vary from, at least, 6.25 ng/μl to 150 ng/μl. The wide range in lysate concentration tolerated is encouraging for probable batch-to-batch variation. It is expected that different batches of AR lysate will vary in AR content. To test the effect of AR lysate variation on the reliability of the androgen screening assay, two commercially sourced OriGene AR lysates (#OA741, #O11311) were compared. These data are presented in FIG. 20, and show that both AR lysates suppressed T7 RNA polymerase activity.

To further explore inter-variability amongst batches, the androgen screening assay was performed with in-house prepared AR lysate preparations (refer to Example 8, below). HEK293 cells stably transformed with a human AR expression plasmid were cultured and from these cells, a cell lysate, a cytoplasm- and a nuclear extract were prepared. The cytoplasm- and nuclear extracts were prepared as AR is sequestered in the cytoplasm and nucleus so it was hypothesized that concentrated AR in these extracts would perform better than total cell lysate. FIG. 21 shows that all of the preparations functioned in the androgen screening assay. However, the cell lysate was the best performer as evidenced by the highest suppression of T7 activity (highest slope, FIG. 21). Cell lysates are the easier and cheapest to prepare so the result is helpful in achieving a cost-effective source of AR.

Given that the in-house HEK293 cell lysate was functional in the androgen screening assay, the next series of experiments returned to the question of inter-variability. Six independent in-house HEK293 cell lysates were prepared, and tested alongside the recombinant AR protein, in the androgen screening assay. All six batches of HEK293 lysate functioned in the assay (FIG. 22). The inter-variability was shown to be 16%.

An important consideration with the use of the cell lysate is that it may contain a number of endogenous SHRs, including glucocorticoid receptor (GR). GR can cross-react with AR at AREs because GR and AR both recognize similar response elements. However, GR should not be activated by testosterone in the androgen screening assay, although it is possible that during the culture of the HEK293 cells endogenous ligands could have activated GR. If ligand-activated GR is present this could produce a false positive result for the androgen screening assay.

To test if ligand-activated GR was present in the HEK293 AR lysates, the androgen screening assay was activated with a common glucocorticoid, dexamethasone. FIG. 23 shows that the androgen screening assay was not responsive to dexamethasone at 1 μM or 100 nM. This provides assurance that there is no GR/AR cross-reactivity in the androgen screening assay when AR lysate is used from HEK293 cells.

Another potential confounder of the androgen screening assay is estradiol (E2). E2 at extremely high, non-physiological, concentrations can activate AR. Standard cell culture conditions should not produce high levels of E2, or any other estrogen. However, to determine the responsiveness of the androgen screening assay to high concentrations of E2, reactions were performed with AR lysate and activated by 1 nM E2. FIG. 23 shows that E2 elicited no response showing that the androgen screening assay response to testosterone is due to the androgen and not to estradiol that may be present in the cell lysate.

The major endogenous androgen is testosterone. The AR lysate-based androgen screening assay responds well to testosterone. There are synthetic steroids, called anabolic androgenic steroids (AAS) and synthetic androgenic molecules, called selective androgen receptor modulators (SARMs). The androgen screening assay with the cell lysate as source of AR was tested for its ability to detect the synthetic steroid, 11keto-testosterone, and the SARM, andarine. The results presented in FIG. 24 show that the androgen screening assay established with the AR lysate was able to respond to the androgens tested.

The androgen screening assay has been designed to detect not only purified compounds but also the relative androgen bioactivity of biological samples. To show that the AR lysate-based androgen screening assay could differentiate relative androgen bioactivity, two equine plasma samples of known high and low values were tested. The androgen screening assay was established with AR lysate at 50 ng/reaction and 15% (v/v) plasma. The results show that the AR lysate-androgen screening assay could detect a difference between the two equine plasma samples and was, at least, equal to the recombinant AR (n=1, FIG. 25).

In humans, circulating androgens are higher in males than females by an average of 10-20-fold. A commercially available human male serum (single donor) was compared to a commercially available human female serum (single donor). The androgen screening assay was used with AR lysate at 50 ng/reaction. The assay was performed using 15% v/v serum samples. The results show that male serum more strongly suppressed T7 activity relative to the female serum (FIG. 26).

In summary, the AR lysate is able to mimic recombinant AR in the androgen screening assay. The AR lysate is responsive to both purified androgen compounds such as designer androgens and SARMs as well as endogenous androgens present in the biological matrices such as plasma and serum. The use of AR lysate markedly decreases the potential cost of the androgen screening assay because there is no need for extensive and expensive purification steps.

There are additional advantages to using AR lysate:
(i) Cell type advantage: the cell lysate will contain AR coregulator proteins. There are over 30 different coregulator proteins for AR. A subset of these are core and are expressed in all cell types. Another subset are specific to cell type. It is therefore likely that an AR lysate prepared from a particular cell type will be more sensitive than the AR lysates used to obtain the data thus far.
(ii) AR modification advantage: the cell lysate can be prepared from transient or stable expression of an AR expression plasmid. This will allow for an AR lysate to be used in the androgen screening assay that has, for example, as stronger binding affinity for androgens. Alternatively, the AR can be modified such that it represents androgen insensitivity syndrome, or partial androgen insensitivity syndrome, allowing for a genetic screen to be established.
(iii) Protein expression advantage: the cell lysate can be prepared from co-transformed cells that, for e.g., express AR and HSP90, or other coregulator proteins. These coregulator proteins can help modulate AR behavior in the androgen screening assay.

### Example 8

Production of Androgen Receptor Cell Lysate in Hek Cell Lines HEK293 cells stably transformed with a human AR cDNA expression plasmid were cultured to 90% confluence in DMEM media supplemented with 10% fetal bovine serum, 1% penicillin/streptomycin and 5.5 μg/mL puromycin. Cells were harvested by washing 1×PBS and then scraping cells with a 1.7 ml Eppendorf tube. Cells are centrifuged at 4° C. at 800×g for 5 mins to pellet the cells. Wash cells with 500 ul PBS by resuspension followed by centrifugation at 500×g for 2-3 mins at 4° C. The cytoplasmic and nuclear extract protocol was then as described by the ThermoFisher Scientific NE-PER Nuclear and Cytoplasmic Extraction Reagents kit (ThermoFisher Scientific #78835). For the total cell extract, the cell pellet was resuspended in ice-cold RIPA buffer (50 mM Tris-HCl pH7.4, 150 mM NaCl, 0.1% SDS, 1% Triton X-100, 1 mM EDTA). Every 5 ml RIPA buffer was supplemented with the inhibitors, 200 ul cOmplete (cOmplete Protease Inhibitor Cocktail, Sigma-Aldrich, 25×), 500 μl PhosSTOP (PhosSTOP Phosphatase Inhibitor Cocktail, Sigma-Aldrich, 10×) and 50 μuL phenylmethylsulfonylfluoride (Sigma-Aldrich, 100 mM). The cells were incubated in RIPA lysis buffer for 30 mins, vortexed then centrifuged at 14,000×g for 10 mins at 4° C. The cell lysate was aliquoted into 1.5 ml Eppendorf tubes for storage at −20° C. the cell lysate is diluted in Protein Stabilizing Cocktail for assays.

### Example 9

### T7 Promoter Architecture Optimization

The next series of experiments tested promoter variations of T7 RNA polymerase. T7 RNA polymerase initiates RNA transcription by binding to a consensus, specific DNA binding site. The binding site, or promoter, can be just 18 bp (SEQ ID NO: 82). For example, this 18 bp sequence is routinely added to a DNA template by PCR, when the DNA template is to be used to PCR to produce RNA.

The wild-type T7 binding site is 23 bp, with the extra base pairs included at the 3′ end (SEQ ID NO: 83).

In the literature, variant promoters have been described whereby an altered DNA sequence leads to increased T7 activity. For example, the wild-type 23 bp 3′ sequence has been modified and such modification reportedly increases T7 promoter activity by ˜ 2-fold (SEQ ID NO: 84). In another example, a substantially longer promoter sequence with modifications to both the 5′ and 3′ sequence reportedly increased T7 promoter activity by up to 23-fold (SEQ ID NO: 84) (Moll et al. (2004) Anal Biochem 334:164-174).

TABLE 13
Sequence compositions for SEQ ID Nos: 82-85

| Sequence ID | Sequence |
|---|---|
| SEQ ID NO: 82 | TAATACGACTCACTATAG |
| SEQ ID NO: 83 | TAATACGACTCACTATAGGGAGA |
| SEQ ID NO: 84 | TAATACGACTCACAATCGCGGAG |
| SEQ ID NO: 85 | GGAGGCCGGAGAATTGTAATACGACTCACTATAGGGAGACGCGTGT |

FIG. 27 shows that T7 promoter activity for the 18 bp (SEQ ID NO: 82), 23 bp (wild-type, SEQ ID NO: 83) and the 23 bp (3′ modified, SEQ ID NO: 84) DNA templates. SEQ ID NO: 82 output is the highest, with wildtype SEQ ID NO: 83 poor by comparison. SEQ ID NO: 84 showed the reported 2-fold increase over the SEQ ID NO: 83 however it too remained inferior to SEQ ID NO: 82. SEQ ID NO: 85, however, was superior to SEQ ID NO: 82, with far higher fluorescence output (comparative data not shown in FIG. 27).

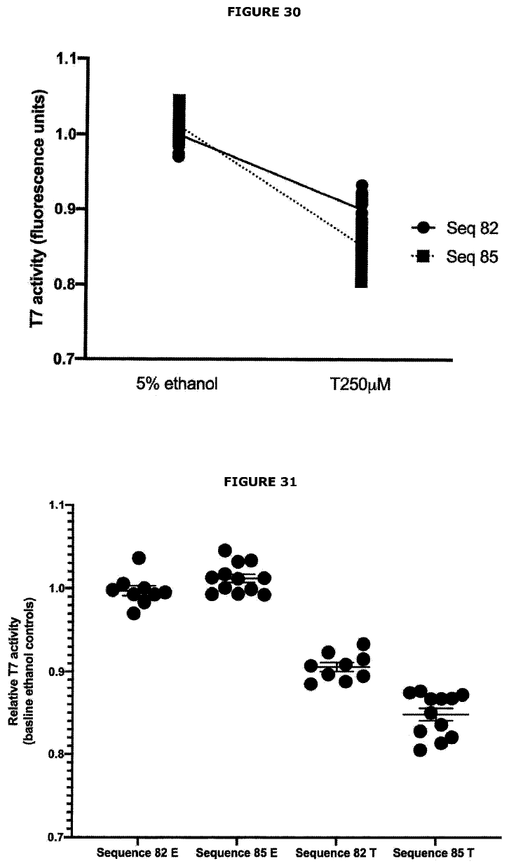

FIG. 18 shows the androgen screening assay results for SEQ ID NOs: 82-85. SEQ ID NO: 82 is clearly the best sequence, relative to SEQ ID NO: 83 and SEQ ID NO: 84, for detecting a blockade of T7 activity by testosterone-activated AR. However, FIG. 29 shows that SEQ ID NO: 85 was superior to SEQ ID NO: 82 for supporting T7 activity. Further, FIG. 30 shows SEQ ID NO: 85 was more effective than SEQ ID NO: 82 in blocking T7 RNA polymerase, with a significant difference decreased from SEQ ID NO: 82 (p<0.001, one way ANOVA with Tukeys post-hoc test).

In summary, SEQ ID NO: 85 has been shown to be a stronger T7 promoter for use in the androgen screening assay.

The specificity of SEQ ID NO: 85-based androgen screening assay was tested by evaluating the response to estradiol and dexamethasone. FIG. 32 showed that T7 SEQ ID NO: 85 did not respond to either steroid. This was shown for SEQ ID NO: 85 whether the androgen screening assay was established with recombinant AR or AR cell lysate (FIG. 33).

Given that SEQ ID NO: 85-based reactions produced more fluorescence output, the reactions were next interrogated at the molecular level. The initial rate of reaction to produce MangoII (fluorescence output) is founded on the key event of T7 to DNA binding. The binding of the polymerase triggers subsequent initiation/elongation steps. This entire event is highly dependent on the T7 promoter sequence, the amount of T7 and the DNA concentration. It follows that if T7 binds the DNA sequence rapidly and efficiently, the transcription of the RNA will be higher.

Ideally, the optimal reaction would have every DNA template bound by a T7 RNA polymerase. However, AR levels also need to be considered in the androgen screening assay because if AR levels are too low, and T7 exceeds AR, then there will be excess DNA template bound by T7 RNA polymerase, that will allow T7 transcription to occur unhindered. A subsequent high baseline could mask limited AR suppression of T7.

To optimize the androgen screening assay, an AR:T7/DNA ratio of >1 is desirable, with the T7:DNA ratio being as ≥1.

FIG. 34 shows that the slope of the reaction with DNA template T7/SEQ ID NO: 85 is higher than that measured for the reaction with DNA template T7/SEQ ID NO: 82. The greater slope suggests that more DNA templates are being utilized by T7 RNA polymerase when the DNA template encodes the SEQ ID NO: 85 T7 RNA promoter. This is most likely because of the high binding affinity of T7 for the promoter defined by SEQ ID NO: 85.

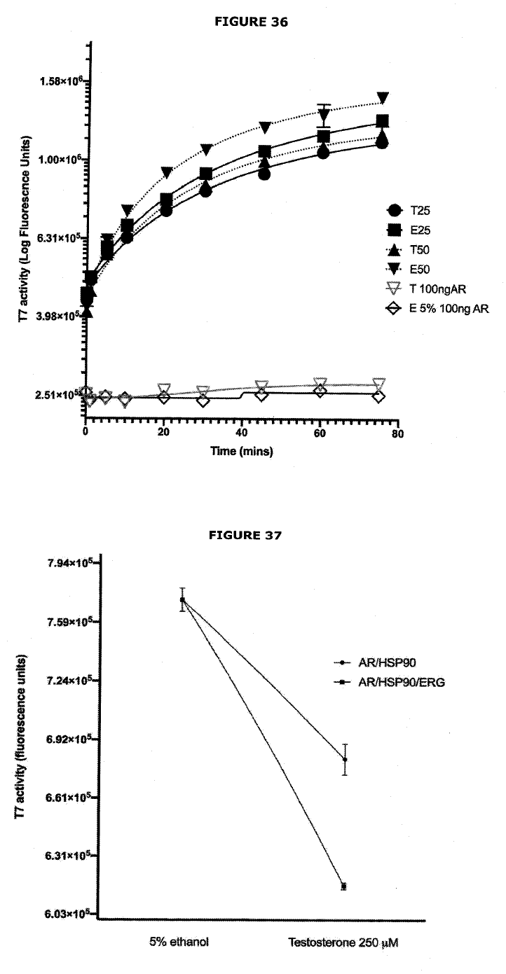

In the reaction described for FIG. 34 above, the DNA template concentration was 100 ng. It may be that the AR:T7/DNA ratio was less than 1, as the ability of testosterone-activated AR to suppress T7 RNA polymerase activity was limited. Therefore, to lower the ratio of AR:T7/DNA, the DNA SEQ ID NO: 85 template per reaction was halved to 50 ng while holding T7 RNA polymerase and AR concentrations steady to determine the optimal concentration for this DNA sequence. FIG. 35 shows that by decreasing DNA template concentration there was a significant reduction in T7 RNA polymerase activity. Therefore at the DNA concentration of 50 ng there is excess T7RNA polymerase:DNA (ratio >1). Excess T7 to DNA means that potentially all DNA templates could have bound T7 RNA polymerase. Further to this, if AR concentration exceeds DNA template then potentially there is sufficient AR (if fully activated) to block all T7 RNA polymerase activity. To test for the optimal AR concentration to block the T7:DNA ratio, AR protein was titrated from 25 ng to 100 ng per reaction. FIG. 36 shows that a concentration of 25 ng AR and 50 ng AR is strong enough to detect AR blockade of T7 RNA polymerase. FIG. 36 also shows that if 100 ng AR is added to the reaction, there is complete blockade of T7 RNA polymerase. Note that the 2:1 ratio of HSP90:AR was maintained throughout these experiments.

In summary, T7SEQ ID NO: 85-androgen screening assay allows for a faster, reproducible test. The reaction time has reduced from 150 mins to 40 mins, with greater change from vehicle controls.

### Example 10

### Interrogation of Alternatively Sourced Cell Lysate

To explore the use of alternate host cells, the androgen screening assay was performed with in-house (inventor's laboratory) AR lysate preparations from AR-expressing Pc-3 cells. Pc-3 cells were originally isolated from bone metastasis of grade IV prostatic adenocarcinoma and reportedly express a range of AR coregulator proteins, including GRIP1, BRCA1, and Zac1 (PMID: 15264248). From Pc-3 cells, cell extracts were prepared and utilized as the source of AR in the cell-free reactions.

For a cell-free reaction, a master mix was prepared with components added in this order: NTP buffer (NEB2052AVIAL component of E2050S), 50 ng DNA template (Seq. ID NO:85), Pc-3 lysate (40 ng), HSP90 (80 ng), testosterone, andarine or ethanol as vehicle control), and T7 RNA polymerase mix. The master mix was gently mixed by pipetting up and down and incubated at 37° C. for 40 minutes. The detection buffer was then added consisting of TO1B (100 nM final concentration) in a 200 mM KCl, 10 mM, Na2HPO4, 0.05% Tween 20 and pH 7.2 solution. The fluorescence was measured using a Spectramax i3× (Molecular Devices) plate reader with excitation 510 nm and emission 535 nm (bandwidth 15 nm). FIG. 40 shows that Pc-3 lysate as a source of AR and its coregulators was equal to an in-house preparation or a commercially sourced (Origene) AR-expressing HEK293 lysate. All lysates were equal in efficacy relative to recombinant AR (Creative Biomart). For these reactions, T7-generated RNA Mango II was evidenced by the ethanol control (set as 100% T7 activity). Testosterone (black columns, 250 μM) or andarine (white columns, 1 μM) was added to activate lysate AR.

These data indicate that cell lysate from AR-expressing cells functions efficiently in the cell-free reactions and can be used to substitute recombinant AR.

To test the sensitivity of Pc-3 AR lysate-driven androgen detection, the reactions used above were performed with decreasing concentrations of testosterone, with the exception that the reactions were established with 25 ng Pc-3 AR lysate. FIG. 41 shows that testosterone could be detected in a dose-dependent fashion.

To test that the ligand-dependent induction of the AR lysate-driven reactions was not just specific to testosterone, a number of anabolic androgenic steroids or alternate testosterone preparations that are commonly detected as used by athletes were substituted for testosterone as the activator of AR.

To test different androgens, a master mix was prepared with components added in this order: NTP buffer (NEB2052AVIAL component of E2050S), 50 ng DNA template (SEQ ID NO: 85), Pc-3 lysate (40 ng), HSP90 (80 ng), and T7 RNA polymerase mix. The master mix was gently mixed by pipetting up and down and aliquoted into individual wells. The steroids being tested were then added to a final concentration of 100 μM and incubated at 37° C. for 40 minutes. The detection buffer was then added consisting of TO1B (100 nM final concentration) in a 200 mM KCl, 10 mM, Na₂HPO₄, 0.05% Tween 20 and pH 7.2 solution. The fluorescence was measured using a Spectramax i3× (Molecular Devices) plate reader with excitation 510 nm and emission 535 nm (bandwidth 15 nm).

FIG. 42 shows that Pc-3 AR lysate was able to detect a wide range of androgenic structures, as represented by the different anabolic androgenic steroids tested.

Finally, FIG. 43 shows that the Pc-3 AR lysate was also able to detect testosterone when presented in ester forms. Esters allow the testosterone molecule to be more soluble in oil, the preferred injectable form of testosterone.

### Example 11

### Spacer Length—Further Interrogation

Applicants previously demonstrated that a spacer length of 15 bp (SEQ ID NO: 86) was better able to support testosterone-activated AR blockade of T7 RNA polymerase activity (FIG. 39) when compared to a spacer length of 9 bp (SEQ ID NO: 87) or 12 bp (SEQ ID NO: 74). Moreover, this improvement in blockade occurred despite the relative equal capacity to support overall T7 RNA polymerase activity, at least for the 12 bp spacer. To interrogate whether longer or shorter spacer lengths could better support testosterone-activated AR blockade of T7 RNA polymerase activity, a number of spacer lengths were evaluated for the ethanol (maximal T7 RNA polymerase activity) to testosterone (blockade of T7 RNA polymerase activity) ratio. The T7 RNA polymerase promoter defined by SEQ ID NO: 85 was used for all DNA constructs in this series of experiments, and a spacer of 2 bp (SEQ ID NO: 88), 12 bp (SEQ ID NO: 74), 15 bp (SEQ ID NO: 86), 18 bp (SEQ ID NO: 89), 21 bp (SEQ ID NO: 90), 24 bp (SEQ ID NO: 91) or 27 bp (SEQ ID NO: 92) was inserted before the single ARE site (for sequence see Table 14). The ARE site was located upstream of the 32merF30scaffoldMangoII reporter construct.

To test the different DNA templates, a master mix was prepared with components added in this order: in-house reaction buffer, NTP mix (1 mM), DNA template (50 ng), Pc-3 lysate (50 ng), HSP90 (100 ng), testosterone (250 μM) or ethanol (5%). The master mix was gently mixed by pipetting up and down and aliquoted into individual wells and NEB Hi-T7 T7 RNA polymerase (50U) was added and reactions incubated at 50° C. for 40 minutes. The detection buffer was then added consisting of Y03 (100 nM final concentration) in a 200 mM KCl, 10 mM Na₂HPO₄, 0.05% Tween 20 and pH 7.2 solution. The fluorescence was measured using a Spectramax i3× (Molecular Devices) plate reader with excitation 595 nm and emission 620 nm (bandwidth 15 nm).

TABLE 14
Altering the T7 to ARE spacer length of the DNA template construct

| | | |
|---|---|---|
| SEQ ID NO: 86 | 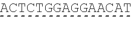 | 15 bp |
| SEQ ID NO: 74 | 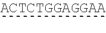 | 12 bp |
| SEQ ID NO: 89 | 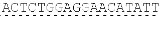 | 18 bp |
| SEQ ID NO: 90 | 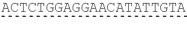 | 21 bp |
| SEQ ID NO: 91 | 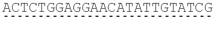 | 24 bp |
| SEQ ID NO: 92 | 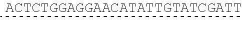 | 27 bp |
| SEQ ID NO: 88 | 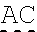 | 2 bp |

FIG. 44 shows that spacer lengths of 12 bp and 24 bp were less able to detect testosterone. A spacer length of 18 bp and 21 bp showed decreased testosterone detection, although the E:T ratio was not significantly different from 15 bp. A spacer length of 27 bp showed equal ability to detect testosterone. Interestingly, a 2 bp spacer was the most effective in detecting testosterone, as evidenced by a greater blockade of T7 RNA polymerase activity. It is likely that a 2 bp spacer allows AR to physically block T7 RNA polymerase activity, i.e. does not allow T7 RNA polymerase to bind and/or begin transcription.

Irrespective, each of the different spacers used in these experiments were able to detect testosterone, adding further support to the feature of a spacer length defined by between about 2 and about 32 nucleotides.

### Example 12

### Interrogating the Nucleotide Composition of the Hormone Response Element and Spacer

The signals that influence the level of transcriptional output include the sequence composition of cis-regulatory elements, including the steroid hormone response elements. For AR, the sequence of the DNA binding motif modulates the receptor's activity. The ARE comprises two inverted repeats of two half-sites of 6 bp separated by a 3 bp spacer. This 15 bp standard ARE is of the consensus sequence AGAACAGCCTGTTCT (SEQ ID NO: 93). The AGAACA (SEQ ID NO: 98) and TGTTCT (SEQ ID NO: 99) sequences represent where the AR dimer binds to the double stranded DNA, however the immediate flanking base pairs can influence activity of steroid hormone receptors, as can the make-up of the 3 bp spacer.

[SEQ ID NO: 14]
5′GGAGGCCGGAGAATTGTAATACGACTCACTATAGGGAGACGCGTGTA
CTCTGGAGGAATGGAGAACAGCCTGTTCTCCATTGCCATGTGTATGTGG
GTACGAAGGAGAGGAGAGGAAGAGGAGAGTACCCACATACTCTGATGAT
CCTTCGGGATCATTCATGGCAATCTAGGA3′

In our exemplary sequence of T7-ARE-MangoII, the ARE (solid underline) is flanked by a G/C combination. To understand whether altering the flanking base pairs to A/T rather than G/C improves the efficacy of the androgen screening assay, SEQ ID Nos: 94 and 95 were tested (Table 15).

In addition to the flanking sequences, the 3 bp spacer sequence can alter steroid hormone receptor activity. To determine if altering the G to T or C to A influences the efficacy of the androgen screening assay, SEQ ID Nos: 96 and 97 were tested (Table 15).

TABLE 15
Altering the ARE spacer and flanking base pairs from G/C to A/T

| | | |
|---|---|---|
| SEQ ID NO: 94 | AAGAACAGCCTGTTCTA | Flanking sequences to A |
| SEQ ID NO: 95 | TAGAACAGCCTGTTCTT | Flanking sequences to T |
| SEQ ID NO: 96 | AGAACATCCTGTTCT | Spacer sequence G to T |
| SEQ ID NO: 97 | AGAACATCATGTTCT | Spacer sequence G to T and |
| | | C to A |

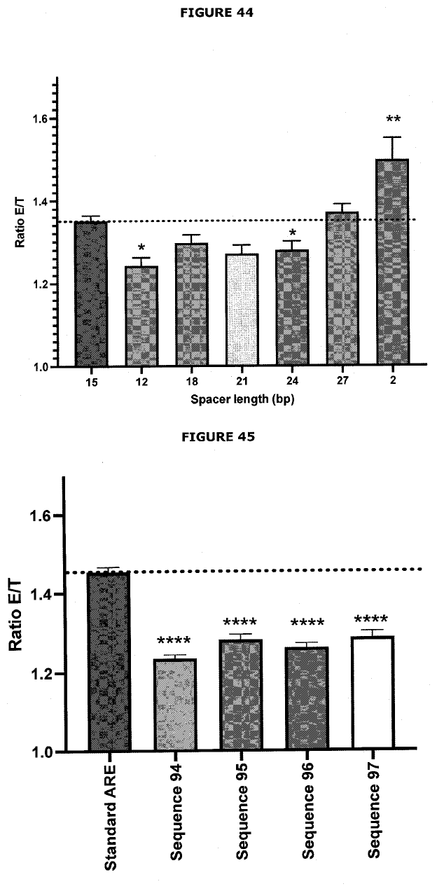

FIG. 45 shows any alteration to the consensus ARE sequence or the standard flanking region decreased the efficacy of the androgen screening assay. These data show that altering the flanking region from G/C to A/T led to a decrease in the ability of testosterone-activated AR to block T7 RNA polymerase transcriptional activity. This is likely due to a decrease in the binding affinity between the altered DNA sequence and the AR DNA binding domain.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts disclosed herein may be resorted to by those skilled in the art, and that such modifications and variations are considered to be within the scope of this invention as described herein, and as defined by the appended claims.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other examples are within the following claims. In addition, where features or aspects of the invention are described in terms of Markush groups, those skilled in the art will recognize that the invention is also thereby described in terms of any individual member or subgroup of members of the Markush group.

1. A test kit for screening a test sample for the presence of a ligand capable of eliciting a steroid hormone genomic response, the test kit comprising:
(i) a cell lysate comprising a steroid hormone receptor that is capable of forming a ligand-receptor complex with a ligand from the test sample; and
(ii) a nucleic acid molecule comprising:
(a) a polymerase promoter sequence comprising or consisting in SEQ ID NO: 85;
(b) a response element that is capable of being bound by the ligand-receptor complex; and
(c) a reporter construct
where the response element (b) is located between the promoter sequence (a) and the reporter construct (c), and (a), (b) and (c) are operably linked; and
(iii) optionally, a T7 RNA polymerase.

2. The test kit according to claim 1, further comprising at least one steroid hormone receptor coactivator selected from erythroblast transformation-specific transcription factor ERG; one or more p160 coactivators inclusive of steroid receptor coactivators, SRC-1, SRC-2, SRC-3; Vav3 a Rho GTPase guanine nucleotide exchange factor; E2F1; ATAD2; CBP/p300; Leupaxin; FHL2; the ARA family of proteins; GRIP1; BRAC1; and Zac1.

3. The test kit according to claim 1 or claim 2, further comprising a steroid hormone receptor corepressor comprising at least one of:
(i) heat shock protein 90 (HSP90);
(ii) a complex of HSP90 and heat shock protein 70 (HSP70);
(iii) a complex of HSP90, HSP70 and heat shock protein 40 (HSP40);
(iv) a complex of HSP90, HSP70, HSP40 and p23;
(v) a complex of HSP90, HSP70, HSP40, p23 and heat shock protein organizing protein (Hop);
(vi) a complex of HSP90, HSP70, HSP40, p23, Hop and 48 kD Hip protein (Hip);
(vii) a complex of HSP90, HSP70, HSP40, p23, Hop, Hip and p60
(viii) a complex of HSP90, HSP70, HSP40, p23, Hop, Hip, p60 and FKBP52; and
(ix) any combination of (i) to (viii).

4. The test kit according to any one of claims 1 to 3, wherein the nucleic acid further comprises a spacer (e) located between the polymerase promoter sequence (a) and the response element (b).

5. The test kit according to claim 4, wherein the spacer is between about 2 and about 32 nucleotides in length.

6. The test kit according to claim 4 to claim 5, wherein the spacer is:
(i) about 2 nucleotides in length;
(ii) about 15 nucleotides in length; or
(ii) about 27 nucleotides in length

7. The test kit according to any one of claims 1 to 6, wherein the nucleic acid molecule further comprises at least one binding site (d) that binds to at least one ERG coactivator protein.

8. The test kit according to any one of claims 1 to 7, wherein the reporter construct comprises a sequence encoding an RNA aptamer capable of binding to a fluorophore.

9. The test kit according to claim 8, wherein the RNA aptamer is Mango II or iSpinach.

10. The test kit according to claim 9, which test kit further comprises the F30 scaffold.

11. The test kit according to any one of claims 1 to 10, wherein the cell lysate is derived from a host cell expressing a plasmid encoding the steroid hormone receptor and/or at least one steroid hormone receptor coactivator or at least one steroid hormone receptor corepressor.

12. The test kit according to any one of claims 1 to 11, which further comprises ribonucleoside triphosphates.

13. The test kit according to any one of claims 1 to 14, wherein the steroid hormone receptor is selected from the group consisting of androgen receptor (AR); estrogen receptor alpha (ER-α) and estrogen receptor beta (ER-β); progesterone receptor A (PRA) and progesterone receptor B (PRB); mineralocorticoid receptor (MR); and glucocorticoid receptor (GR).

14. The test kit according to any one of claims 1 to 13, wherein the response element is selected from:
a. an androgen response element (ARE) including, but not limited to, a sequence comprising 5′-AGAACAnnnTGTTCT-3′ (SEQ ID NO: 4), wherein n is A, T, G or C;
b. an estrogen response element (ERE) including, but not limited to, a sequence comprising 5′-AGGTCAnnnTGACCT-3′ (SEQ ID NO: 8), wherein n is A, T, G or C;
c. a progesterone response element (PRE) including, but not limited to, a sequence comprising 5′-GGTACAAACTGTTCT-3′ (SEQ ID NO: 10);
d. a mineralocorticoid response element (MRE) including, but not limited to, a sequence comprising 5′-AGAACAnAATGTTCT-3′ (SEQ ID NO: 12), wherein n is A, T, G or C; and
e. a glucocorticoid response element (GRE) including, but not limited to, a sequence comprising 5′-AGAACAnAATGTTCT-3′ (SEQ ID NO: 12), wherein n is A, T, G or C.

15. A method for determining the doping status of an athlete by combining the components of the test kit according to any one of claims 1 to 14 with a sample obtained from the athlete to ascertain if the sample comprises a ligand sufficient to bind to and activate a steroid hormone receptor and cause a change in a physical property of the reporter construct, wherein a change in a physical property of the reporter construct provides information about the doping status of the athlete.


SEQUENCE LISTING (100 sequences)

SEQ ID NO: 1 - DNA, 18 bp, Artificial Sequence
  T7 RNA polymerase promoter/initiator sequence
taatacgactcactatag

SEQ ID NO: 2 - DNA, 18 bp, Artificial Sequence
  T3 RNA polymerase promoter/initiator sequence
aattaaccctcactaaag

SEQ ID NO: 3 - DNA, 18 bp, Artificial Sequence
  SP6 RNA polymerase promoter/initiator sequence
atttaggtgacactatag

SEQ ID NO: 4 - DNA, 15 bp, Artificial Sequence
  Androgen Response Element 1 - Sense Strand
  misc_feature (7)..(9): n is a, c, g, or t
agaacannntgttct

SEQ ID NO: 5 - DNA, 15 bp, Artificial Sequence
  Androgen Response Element 1 - Antisense Strand
  misc_feature (7)..(9): n is a, c, g, or t
tcttgtnnnacaaga

SEQ ID NO: 6 - DNA, 15 bp, Artificial Sequence
  Androgen Response Element 2 - Sense Strand
  misc_feature (7)..(9): n is a, c, g, or t
ggtacannntgttct

SEQ ID NO: 7 - DNA, 15 bp, Artificial Sequence
  Androgen Response Element 2 - Antisense Strand
  misc_feature (7)..(9): n is a, c, g, or t
ccatgtnnnacaaga

SEQ ID NO: 8 - DNA, 15 bp, Artificial Sequence
  Estrogen Response Element - Sense Strand
  misc_feature (7)..(9): n is a, c, g, or t
aggtcannntgacct

SEQ ID NO: 9 - DNA, 15 bp, Artificial Sequence
  Estrogen Response Element - Sense Strand
  misc_feature (7)..(9): n is a, c, g, or t
tccagtnnnactgga

SEQ ID NO: 10 - DNA, 15 bp, Artificial Sequence
  Progesterone Response Element - Sense Strand
ggtacaaactgttct

SEQ ID NO: 11 - DNA, 15 bp, Artificial Sequence
  Progesterone Response Element - Antisense Strand
ccatgtttgacaaga

SEQ ID NO: 12 - DNA, 15 bp, Artificial Sequence
  Glucocorticoid/Mineralocorticoid Response Element - Sense Strand
  misc_feature (7)..(7): n is a, c, g, or t
agaacanaatgttct

SEQ ID NO: 13 - DNA, 15 bp, Artificial Sequence
  Glucocorticoid/Mineralocorticoid Response Element - Antisense Strand
  misc_feature (7)..(7): n is a, c, g, or t
tcttgtnttacaaga

SEQ ID NO: 14 - DNA, 174 bp, Artificial Sequence
  T7i-ARE-F30MangoII
ggaggccggagaattgtaatacgactcactatagggagacgcgtgtactctggaggaatggagaacagcctgttctccattgccatgtgtatgtgggtacgaaggagaggagaggaagaggagagtacccacatactctgatgatccttcgggatcattcatggcaatctagga

SEQ ID NO: 15 - DNA, 170 bp, Artificial Sequence
  T7i-ERE-F30MangoII
ggaggccggagaattgtaatacgactcactatagggagacgcgtgtactctggaggaacaggtcagcatgacctgttgccatgtgtatgtgggtacgaaggagaggagaggaagaggagagtacccacatactctgatgatccttcgggatcattcatggcaatctagga

SEQ ID NO: 16 - DNA, 168 bp, Artificial Sequence
  T7i-PRE-F30MangoII
ggaggccggagaattgtaatacgactcactatagggagacgcgtgtactctggaggaaggtacaaactgttctttgccatgtgtatgtgggtacgaaggagaggagaggaagaggagagtacccacatactctgatgatccttcgggatcattcatggcaatctagga

SEQ ID NO: 17 - DNA, 168 bp, Artificial Sequence
  T7i-MRE-F30MangoII
ggaggccggagaattgtaatacgactcactatagggagacgcgtgtactctggaggaatgtacaggatgttctttgccatgtgtatgtgggtacgaaggagaggagaggaagaggagagtacccacatactctgatgatccttcgggatcattcatggcaatctagga

SEQ ID NO: 18 - DNA, 168 bp, Artificial Sequence; identical to SEQ ID NO: 17
  T7i-GRE-F30MangoII

SEQ ID NO: 19 - DNA, 109 bp, Artificial Sequence
  T7i-ARE-MangoII
ggaggccggagaattgtaatacgactcactatagggagacgcgtgtactctggaggaatggagaacagcctgttctccatacgaaggagaggagaggaagaggagagta

SEQ ID NO: 20 - DNA, 105 bp, Artificial Sequence
  T7i-ERE-MangoII
ggaggccggagaattgtaatacgactcactatagggagacgcgtgtactctggaggaacaggtcagcatgacctgtacgaaggagaggagaggaagaggagagta

SEQ ID NO: 21 - DNA, 103 bp, Artificial Sequence
  T7i-PRE-MangoII
ggaggccggagaattgtaatacgactcactatagggagacgcgtgtactctggaggaaggtacaaactgttcttacgaaggagaggagaggaagaggagagta

SEQ ID NO: 22 - DNA, 103 bp, Artificial Sequence
  T7i-MRE-MangoII
ggaggccggagaattgtaatacgactcactatagggagacgcgtgtactctggaggaatgtacaggatgttcttacgaaggagaggagaggaagaggagagta

SEQ ID NO: 23 - DNA, 103 bp, Artificial Sequence; identical to SEQ ID NO: 22
  T7i-GRE-MangoII

SEQ ID NO: 24 - DNA, 148 bp, Artificial Sequence
  T7i-ARE-iSpinach
ggaggccggagaattgtaatacgactcactatagggagacgcgtgtactctggaggaatggagaacagcctgttctccaaggagtacggtgagggtcgggtccagtaggtacgcctactgttgagtagagtgtgggctccgtactccc

SEQ ID NO: 25 - DNA, 144 bp, Artificial Sequence
  T7i-ERE-iSpinach
ggaggccggagaattgtaatacgactcactatagggagacgcgtgtactctggaggaacaggtcagcatgacctgaggagtacggtgagggtcgggtccagtaggtacgcctactgttgagtagagtgtgggctccgtactccc

SEQ ID NO: 26 - DNA, 142 bp, Artificial Sequence
  T7i-PRE-iSpinach
ggaggccggagaattgtaatacgactcactatagggagacgcgtgtactctggaggaaggtacaaactgttctaggagtacggtgagggtcgggtccagtaggtacgcctactgttgagtagagtgtgggctccgtactccc

SEQ ID NO: 27 - DNA, 142 bp, Artificial Sequence
  T7i-MRE-iSpinach
ggaggccggagaattgtaatacgactcactatagggagacgcgtgtactctggaggaatgtacaggatgttctaggagtacggtgagggtcgggtccagtaggtacgcctactgttgagtagagtgtgggctccgtactccc

SEQ ID NO: 28 - DNA, 142 bp, Artificial Sequence; identical to SEQ ID NO: 27
  T7i-GRE-iSpinach

SEQ ID NO: 29 - DNA, 218 bp, Artificial Sequence
  T7i-ARE-F30iSpinach
ggaggccggagaattgtaatacgactcactatagggagacgcgtgtactctggaggaatggagaacagcctgttctccattgccatgtgtatgtgggaggagtacggtgagggtcgggtccagtaggtacgcctactgttgagtagagtgtgggctccgtactccccccacatactctgatgatccttcgggatcattcatggcaatctagatctaga

SEQ ID NO: 30 - DNA, 214 bp, Artificial Sequence
  T7i-ERE-F30iSpinach
ggaggccggagaattgtaatacgactcactatagggagacgcgtgtactctggaggaacaggtcagcatgacctgttgccatgtgtatgtgggaggagtacggtgagggtcgggtccagtaggtacgcctactgttgagtagagtgtgggctccgtactccccccacatactctgatgatccttcgggatcattcatggcaatctagatctaga

SEQ ID NO: 31 - DNA, 212 bp, Artificial Sequence
  T7i-PRE-F30iSpinach
ggaggccggagaattgtaatacgactcactatagggagacgcgtgtactctggaggaaggtacaaactgttctttgccatgtgtatgtgggaggagtacggtgagggtcgggtccagtaggtacgcctactgttgagtagagtgtgggctccgtactccccccacatactctgatgatccttcgggatcattcatggcaatctagatctaga

SEQ ID NO: 32 - DNA, 212 bp, Artificial Sequence
  T7i-MRE-F30iSpinach
ggaggccggagaattgtaatacgactcactatagggagacgcgtgtactctggaggaatgtacaggatgttctttgccatgtgtatgtgggaggagtacggtgagggtcgggtccagtaggtacgcctactgttgagtagagtgtgggctccgtactccccccacatactctgatgatccttcgggatcattcatggcaatctagatctaga

SEQ ID NO: 33 - DNA, 212 bp, Artificial Sequence; identical to SEQ ID NO: 32
  T7i-GRE-F30iSpinach

SEQ ID NO: 34 - DNA, 81 bp, Artificial Sequence
  T3i-ARE-MangoII
aattaaccctcactaaagactctggaggaatggagaacagcctgttctccatacgaaggagaggagaggaagaggagagta

SEQ ID NO: 35 - DNA, 77 bp, Artificial Sequence
  T3i-ERE-MangoII
aattaaccctcactaaagactctggaggaacaggtcagcatgacctgtacgaaggagaggagaggaagaggagagta

SEQ ID NO: 36 - DNA, 75 bp, Artificial Sequence
  T3i-PRE-MangoII
aattaaccctcactaaagactctggaggaaggtacaaactgttcttacgaaggagaggagaggaagaggagagta

SEQ ID NO: 37 - DNA, 75 bp, Artificial Sequence
  T3i-MRE-MangoII
aattaaccctcactaaagactctggaggaatgtacaggatgttcttacgaaggagaggagaggaagaggagagta

SEQ ID NO: 38 - DNA, 75 bp, Artificial Sequence; identical to SEQ ID NO: 37
  T3i-GRE-MangoII

SEQ ID NO: 39 - DNA, 146 bp, Artificial Sequence
  T3i-ARE-F30MangoII
aattaaccctcactaaagactctggaggaatggagaacagcctgttctccattgccatgtgtatgtgggtacgaaggagaggagaggaagaggagagtacccacatactctgatgatccttcgggatcattcatggcaatctagga

SEQ ID NO: 40 - DNA, 142 bp, Artificial Sequence
  T3i-ERE-F30MangoII
aattaaccctcactaaagactctggaggaacaggtcagcatgacctgttgccatgtgtatgtgggtacgaaggagaggagaggaagaggagagtacccacatactctgatgatccttcgggatcattcatggcaatctagga

SEQ ID NO: 41 - DNA, 140 bp, Artificial Sequence
  T3i-PRE-F30MangoII
aattaaccctcactaaagactctggaggaaggtacaaactgttctttgccatgtgtatgtgggtacgaaggagaggagaggaagaggagagtacccacatactctgatgatccttcgggatcattcatggcaatctagga

SEQ ID NO: 42 - DNA, 140 bp, Artificial Sequence
  T3i-MRE-F30MangoII
aattaaccctcactaaagactctggaggaatgtacaggatgttctttgccatgtgtatgtgggtacgaaggagaggagaggaagaggagagtacccacatactctgatgatccttcgggatcattcatggcaatctagga

SEQ ID NO: 43 - DNA, 140 bp, Artificial Sequence; identical to SEQ ID NO: 42
  T3i-GRE-F30MangoII

SEQ ID NO: 44 - DNA, 120 bp, Artificial Sequence
  T3i-ARE-iSpinach
aattaaccctcactaaagactctggaggaatggagaacagcctgttctccaaggagtacggtgagggtcgggtccagtaggtacgcctactgttgagtagagtgtgggctccgtactccc

SEQ ID NO: 45 - DNA, 116 bp, Artificial Sequence
  T3i-ERE-iSpinach
aattaaccctcactaaagactctggaggaacaggtcagcatgacctgaggagtacggtgagggtcgggtccagtaggtacgcctactgttgagtagagtgtgggctccgtactccc

SEQ ID NO: 46 - DNA, 114 bp, Artificial Sequence
  T3i-PRE-iSpinach
aattaaccctcactaaagactctggaggaaggtacaaactgttctaggagtacggtgagggtcgggtccagtaggtacgcctactgttgagtagagtgtgggctccgtactccc

SEQ ID NO: 47 - DNA, 114 bp, Artificial Sequence
  T3i-MRE-iSpinach
aattaaccctcactaaagactctggaggaatgtacaggatgttctaggagtacggtgagggtcgggtccagtaggtacgcctactgttgagtagagtgtgggctccgtactccc

SEQ ID NO: 48 - DNA, 114 bp, Artificial Sequence; identical to SEQ ID NO: 47
  T3i-GRE-iSpinach

SEQ ID NO: 49 - DNA, 190 bp, Artificial Sequence
  T3i-ARE-F30iSpinach
aattaaccctcactaaagactctggaggaatggagaacagcctgttctccattgccatgtgtatgtgggaggagtacggtgagggtcgggtccagtaggtacgcctactgttgagtagagtgtgggctccgtactccccccacatactctgatgatccttcgggatcattcatggcaatctagatctaga

SEQ ID NO: 50 - DNA, 186 bp, Artificial Sequence
  T3i-ERE- F30iSpinach
aattaaccctcactaaagactctggaggaacaggtcagcatgacctgttgccatgtgtatgtgggaggagtacggtgagggtcgggtccagtaggtacgcctactgttgagtagagtgtgggctccgtactccccccacatactctgatgatccttcgggatcattcatggcaatctagatctaga

SEQ ID NO: 51 - DNA, 184 bp, Artificial Sequence
  T3i-PRE- F30iSpinach
aattaaccctcactaaagactctggaggaaggtacaaactgttctttgccatgtgtatgtgggaggagtacggtgagggtcgggtccagtaggtacgcctactgttgagtagagtgtgggctccgtactccccccacatactctgatgatccttcgggatcattcatggcaatctagatctaga

SEQ ID NO: 52 - DNA, 184 bp, Artificial Sequence
  T3i-MRE- F30iSpinach
aattaaccctcactaaagactctggaggaatgtacaggatgttctttgccatgtgtatgtgggaggagtacggtgagggtcgggtccagtaggtacgcctactgttgagtagagtgtgggctccgtactccccccacatactctgatgatccttcgggatcattcatggcaatctagatctaga

SEQ ID NO: 53 - DNA, 184 bp, Artificial Sequence; identical to SEQ ID NO: 52
  T3i-GRE- F30iSpinach

SEQ ID NO: 54 - DNA, 81 bp, Artificial Sequence
  SP6i-ARE-MangoII
atttaggtgacactatagactctggaggaatggagaacagcctgttctccatacgaaggagaggagaggaagaggagagta

SEQ ID NO: 55 - DNA, 77 bp, Artificial Sequence
  SP6i-ERE-MangoII
atttaggtgacactatagactctggaggaacaggtcagcatgacctgtacgaaggagaggagaggaagaggagagta

SEQ ID NO: 56 - DNA, 75 bp, Artificial Sequence
  SP6i-PRE-MangoII
atttaggtgacactatagactctggaggaaggtacaaactgttcttacgaaggagaggagaggaagaggagagta

SEQ ID NO: 57 - DNA, 75 bp, Artificial Sequence
  SP6i-MRE-MangoII
atttaggtgacactatagactctggaggaatgtacaggatgttcttacgaaggagaggagaggaagaggagagta

SEQ ID NO: 58 - DNA, 75 bp, Artificial Sequence; identical to SEQ ID NO: 57
  SP6i-GRE-MangoII

SEQ ID NO: 59 - DNA, 146 bp, Artificial Sequence
  SP6i-ARE-F30MangoII
atttaggtgacactatagactctggaggaatggagaacagcctgttctccattgccatgtgtatgtgggtacgaaggagaggagaggaagaggagagtacccacatactctgatgatccttcgggatcattcatggcaatctagga

SEQ ID NO: 60 - DNA, 142 bp, Artificial Sequence
  SP6i-ERE-F30MangoII
atttaggtgacactatagactctggaggaacaggtcagcatgacctgttgccatgtgtatgtgggtacgaaggagaggagaggaagaggagagtacccacatactctgatgatccttcgggatcattcatggcaatctagga

SEQ ID NO: 61 - DNA, 140 bp, Artificial Sequence
  SP6i-PRE-F30MangoII
atttaggtgacactatagactctggaggaaggtacaaactgttctttgccatgtgtatgtgggtacgaaggagaggagaggaagaggagagtacccacatactctgatgatccttcgggatcattcatggcaatctagga

SEQ ID NO: 62 - DNA, 140 bp, Artificial Sequence
  SP6i-MRE-F30MangoII
atttaggtgacactatagactctggaggaatgtacaggatgttctttgccatgtgtatgtgggtacgaaggagaggagaggaagaggagagtacccacatactctgatgatccttcgggatcattcatggcaatctagga

SEQ ID NO: 63 - DNA, 140 bp, Artificial Sequence; identical to SEQ ID NO: 62
  SP6i-GRE-F30MangoII

SEQ ID NO: 64 - DNA, 120 bp, Artificial Sequence
  SP6i-ARE-iSpinach
atttaggtgacactatagactctggaggaatggagaacagcctgttctccaaggagtacggtgagggtcgggtccagtaggtacgcctactgttgagtagagtgtgggctccgtactccc

SEQ ID NO: 65 - DNA, 116 bp, Artificial Sequence
  SP6i-ERE-iSpinach
atttaggtgacactatagactctggaggaacaggtcagcatgacctgaggagtacggtgagggtcgggtccagtaggtacgcctactgttgagtagagtgtgggctccgtactccc

SEQ ID NO: 66 - DNA, 114 bp, Artificial Sequence
  SP6i-PRE-iSpinach
atttaggtgacactatagactctggaggaaggtacaaactgttctaggagtacggtgagggtcgggtccagtaggtacgcctactgttgagtagagtgtgggctccgtactccc

SEQ ID NO: 67 - DNA, 114 bp, Artificial Sequence
  SP6i-MRE-iSpinach
atttaggtgacactatagactctggaggaatgtacaggatgttctaggagtacggtgagggtcgggtccagtaggtacgcctactgttgagtagagtgtgggctccgtactccc

SEQ ID NO: 68 - DNA, 114 bp, Artificial Sequence; identical to SEQ ID NO: 67
  SP6i-GRE-iSpinach

SEQ ID NO: 69 - DNA, 190 bp, Artificial Sequence
  SP6i-ARE-F30iSpinach
atttaggtgacactatagactctggaggaatggagaacagcctgttctccattgccatgtgtatgtgggaggagtacggtgagggtcgggtccagtaggtacgcctactgttgagtagagtgtgggctccgtactccccccacatactctgatgatccttcgggatcattcatggcaatctagatctaga

SEQ ID NO: 70 - DNA, 186 bp, Artificial Sequence
  SP6i-ERE- F30iSpinach
atttaggtgacactatagactctggaggaacaggtcagcatgacctgttgccatgtgtatgtgggaggagtacggtgagggtcgggtccagtaggtacgcctactgttgagtagagtgtgggctccgtactccccccacatactctgatgatccttcgggatcattcatggcaatctagatctaga

SEQ ID NO: 71 - DNA, 184 bp, Artificial Sequence
  SP6i-PRE- F30iSpinach
atttaggtgacactatagactctggaggaaggtacaaactgttctttgccatgtgtatgtgggaggagtacggtgagggtcgggtccagtaggtacgcctactgttgagtagagtgtgggctccgtactccccccacatactctgatgatccttcgggatcattcatggcaatctagatctaga

SEQ ID NO: 72 - DNA, 184 bp, Artificial Sequence
  SP6i-MRE-F30iSpinach
atttaggtgacactatagactctggaggaatgtacaggatgttctttgccatgtgtatgtgggaggagtacggtgagggtcgggtccagtaggtacgcctactgttgagtagagtgtgggctccgtactccccccacatactctgatgatccttcgggatcattcatggcaatctagatctaga

SEQ ID NO: 73 - DNA, 184 bp, Artificial Sequence; identical to SEQ ID NO: 72
  SP6i-GRE-F30iSpinach

SEQ ID NO: 74 - DNA, 12 bp, Artificial Sequence
  12 bp Spacer Sequence
actctggaggaa

SEQ ID NO: 75 - DNA, 67 bp, Artificial Sequence
  3xTandem Androgen Response Element
aagcttagaacagtttgtaacgagctcgttacaaactgttctagctcgttacaaactgttctaagct

SEQ ID NO: 76 - DNA, 21 bp, Artificial Sequence
  Androgen Response Element
tggagaacagcctgttctcca

SEQ ID NO: 77 - DNA, 95 bp, Artificial Sequence
  MangoII Aptamer + F30 Scaffold
ttgccatgtgtatgtgggtacgaaggagaggagaggaagaggagagtacccacatactctgatgatccttcgggatcattcatggcaatctagga

SEQ ID NO: 78 - DNA, 30 bp, Artificial Sequence
  MangoII Aptamer
tacgaaggagaggagaggaagaggagagta

SEQ ID NO: 79 - DNA, 17 bp, Artificial Sequence
  Estrogen Response Element
caggtcagcatgacctg

SEQ ID NO: 80 - DNA, 6 bp, Artificial Sequence
  Hormone Response Element hexameric palindrome sequence
tgttct

SEQ ID NO: 81 - DNA, 6 bp, Artificial Sequence
  Hormone Response Element hexameric palindrome sequence
tgacct

SEQ ID NO: 82 - DNA, 18 bp, Artificial Sequence
  T7 RNA polymerase promoter/initiator sequence
taatacgactcactatag

SEQ ID NO: 83 - DNA, 23 bp, Artificial Sequence
  T7 RNA polymerase promoter/initiator sequence
taatacgactcactatagggaga

SEQ ID NO: 84 - DNA, 23 bp, Artificial Sequence
  T7 RNA polymerase promoter/initiator sequence
taatacgactcacaatcgcggag

SEQ ID NO: 85 - DNA, 46 bp, Artificial Sequence
  T7 RNA polymerase promoter/initiator sequence
ggaggccggagaattgtaatacgactcactatagggagacgcgtgt

SEQ ID NO: 86 - DNA, 15 bp, Artificial Sequence
  15 bp Spacer Sequence
actctggaggaacat

SEQ ID NO: 87 - DNA, 9 bp, Artificial Sequence
  9 bp Spacer Sequence
actctggag

SEQ ID NO: 88 - DNA, 2 bp, Artificial Sequence
  2 bp Spacer Sequence
ac

SEQ ID NO: 89 - DNA, 18 bp, Artificial Sequence
  18 bp Spacer Sequence
actctggaggaacatatt

SEQ ID NO: 90 - DNA, 21 bp, Artificial Sequence
  21 bp Spacer Sequence
actctggaggaacatattgta

SEQ ID NO: 91 - DNA, 24 bp, Artificial Sequence
  24 bp Spacer Sequence
actctggaggaacatattgtatcg

SEQ ID NO: 92 - DNA, 27 bp, Artificial Sequence
  27 bp Spacer Sequence
actctggaggaacatattgtatcgatt

SEQ ID NO: 93 - DNA, 15 bp, Artificial Sequence
  Androgen Response Element
agaacagcctgttct

SEQ ID NO: 94 - DNA, 17 bp, Artificial Sequence
  Modified Androgen Response Element
aagaacagcctgttcta

SEQ ID NO: 95 - DNA, 17 bp, Artificial Sequence
  Modified Androgen Response Element
tagaacagcctgttctt

SEQ ID NO: 96 - DNA, 15 bp, Artificial Sequence
  Modified Androgen Response Element
agaacatcctgttct

SEQ ID NO: 97 - DNA, 15 bp, Artificial Sequence
  Modified Androgen Response Element
agaacatcatgttct

SEQ ID NO: 98 - DNA, 6 bp, Artificial Sequence
  Androgen Receptor Binding Site
agaaca

SEQ ID NO: 99 - DNA, 6 bp, Artificial Sequence
  Androgen Receptor Binding Site
tgttct

SEQ ID NO: 100 - DNA, 387 bp, Artificial Sequence
  T7i-ARE-F30MangoII-T7i-ERE-iSpinach
ggaggccggagaattgtaatacgactcactatagggagacgcgtgtactctggaggaatggagaacagcctgttctccattgccatgtgtatgtgggtacgaaggagaggagaggaagaggagagtacccacatactctgatgatccttcgggatcattcatggcaatctaggaaaccccgcggggcctttcgggggtctcgcggggttttttgctcggaggccggagaattgtaatacgactcactatagggagacgcgtgtactctggaggaacaggtcagcatgacctgttgccatgtgtatgtgggtacgaaggagaggagaggaagaggagagtacccacatactctgatgatccttcgggatcattcatggcaatctagga